(19) United States
(12) Patent Application Publication
(10) Pub. No.: US 2021/0276987 A1
(43) Pub. Date: Sep. 9, 2021

(54) PHARMACEUTICAL AGENTS FOR USE IN SMOKING AND TOBACCO CESSATION

(71) Applicant: Washington State University, Pullman, WA (US)

(72) Inventors: Philip Lazarus, Pullman, WA (US); Travis Denton, Pullman, WA (US); Gang Chen, Pullman, WA (US); Pramod Srivastava, Pullman, WA (US); Alec Wynd, Pullman, WA (US); Zuping Xia, Pullman, WA (US); Christy Watson, Pullman, WA (US)

(73) Assignee: Washington State University, Pullman, WA (US)

(21) Appl. No.: 17/257,160

(22) PCT Filed: Jul. 3, 2019
(86) PCT No.: PCT/US2019/040579

§ 371 Date: Dec. 30, 2020

Related U.S. Application Data
(60) Provisional application No. 62/693,722, filed on Jul. 3, 2018.

(51) Int. Cl. C07D 409/14; C07D 409/04; C07D 405/14; C07D 207/44; C07D 239/26; A61K 45/06

(52) U.S. Cl. CPC C07D 409/14; C07D 409/04; A61K 45/06; C07D 207/44; C07D 239/26; C07D 405/14

(57) ABSTRACT

The present disclosure describes novel compounds and compositions that reduce nicotine mediated cravings in humans. In embodiments, the novel compounds blocking CYP2A6-meditated nicotine metabolism thereby reducing the need for additional nicotine. Leading to a desirable treatment option in reducing nicotine craving which does not exacerbate the sympathetic response rate caused by the abused substance and which has favorable pharmacodynamics effects.

### CROSS REFERENCE TO RELATED APPLICATIONS

This application claims the benefit of U.S. Provisional Patent Application No. 62/693,722, filed on Jul. 3, 2018, which is hereby incorporated by reference in its entirety.

### TECHNICAL FIELD

The present disclosure relates to novel compounds and compositions for reducing nicotine mediated cravings in mammals. The novel compounds selectively block cytochrome P-450 2A6 (CYP2A6) and/or UDP-glucuronosyltransferase 2B10 (UGT210) meditated nicotine metabolism, thereby reducing the need for additional nicotine.

### BACKGROUND

Considerable research has been directed at nicotine addiction and other related substance abuse. The cost to society is very high from the health costs associated with obesity, tobacco consumption and drug and alcohol abuse. While many individuals choose to lose weight, stop smoking, and/or cease abusing drugs, they frequently relapse into their former patterns of behavior during, or shortly after, they complete their treatment programs. Often, this may be caused by subtle signals in the environment, which initiate cravings in the individual for the substance which they had abused. Accordingly, it is desirable to provide a substance which would reduce the need for nicotine in a predisposed mammal.

U.S. Pat. Nos. 865,026, 940,521, 3,877,468, 3,901,248, and 3,845,217, disclosing nicotine containing chewing gums, state that it seems particularly difficult to find other smoking substitutes equivalent to or as effective as these nicotines containing chewing gums. U.S. Pat. No. 4,579,858 discloses a smoking substitute composition for application directly into the nose, consisting essentially of an aqueous solution of nicotine or a physiologically acceptable acid addition salt thereof, having a pH value of 2 to 6, containing 10 to 0.5% w/v of nicotine calculated as the free base, containing a nasally-acceptable thickening agent, having a viscosity not less than 100 centipoise, and having about 0.5 to 5 mg nicotine per every 0.05 to 0.5 ml thereof and a method of diminishing the desire of a subject to smoke, which comprises the step of administering to the subject intranasally.

U.S. Pat. No. 4,311,691 defines a composition for inhibiting tobacco smoking comprising a gamma pyrone and an inert physiologically acceptable carrier capable of providing sustained release of the gamma pyrone in the mouth over a time period of at least ten (10) minutes, in unit dosage form containing from 20 mg to 300 mg of gamma pyrone per unit dose and a chewing gum composition for inhibiting tobacco smoking comprising a chewing gum base having particulate ethyl maltol distributed uniformly throughout, providing 100 mg to 300 mg ethyl maltol per stick of gum.

U.S. Pat. No. 4,276,890 defines a method of inhibiting tobacco smoking of smokers without physiological symptoms of nicotine withdrawal comprising smoking while awake during the waking hours of the day and administering to such a smoker 500 mg to 1500 mg total daily dose of ethyl maltol or maltol as a gamma pyrone divided into several incremental doses during the waking hours of the day, each incremental dose being retained in the smoker's mouth and released therein over a period of at least 10 minutes, for at least about 5 to 10 days, for a total of about 20 to 30 days or at least until there results either of a gradual decrease in the number of cigarettes smoked and the length of time they are smoked or until such point as the lowered tobacco consumption rate becomes obvious.

Substances which are administered to reduce the need for nicotine should not produce significant physiological effects, such as stimulation of mood or elevate blood pressure or heart rate. This could result in the substitution of one abused substance for another. Compounds that dampen the desire for the abused substance also should not contain the abused substance or have ingredients that exacerbate the physiological symptoms of the abused substance in the event the individual relapses and takes the abused substance. Also, substances administered to reduce craving should not produce significant adverse effects, such as, for example purposes only, dysphoria, restlessness or stiffness.

Smokers adjust their tobacco use to maintain a certain blood and brain level of nicotine. Because of this, modulation of the enzymatic degradation of nicotine may have potential as a cessation strategy. After entering the circulation, nicotine is eliminated mainly by metabolism to its major metabolite, cotinine. Nicotine to cotinine metabolism occurs in a two-step reaction, where nicotine is first oxidized to nicotine-Δ5′(1′)-iminium ion, mainly catalyzed by CYP2A6. UGT2B10 catalyzes the N-glucuronidation of both nicotine and cotinine. Inhibitors of cytochrome P-450 2A6 (CYP2A6) have been synthesized by Yanno et al. (Yanno et al., J. Med. Chem. 2006, 49, 6987-7001). However, Yanno et al. did not identify a potent and selective inhibitor for CYP2A6.

Accordingly, it is desirable to provide a compound and method of treatment which will be active in reducing craving for the abused substance, will not include the substance being abused, does not exacerbate the sympathetic response rate caused by the abused substance, and has favorable pharmacodynamics effects.

### SUMMARY

The present disclosure describes novel compounds that can be used to treat various diseases and disorders associated with nicotine use. For example, the compounds can be used to decrease the normal levels of nicotine metabolism in tobacco users, functionally extending the half-life of nicotine and ultimately prolonging the onset of withdrawal symptoms in an individual dependent on tobacco.

The novel compounds have the following formula:

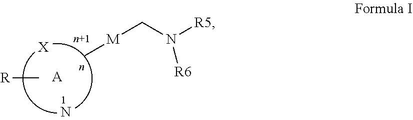

wherein,
A is a heterocycle comprising X, N, and one to 5 carbon atoms, and N is at position 1 on the heterocycle;
X is C, N, O, or S, and X is at any position on the heterocycle that is not occupied by N or a carbon atom bonded to M;
M is a linker, and M is bonded to the carbon atom on the heterocycle at position n,
wherein n is 2, 3, 4, 5, or 6;
R is one or more substituents on A, and at least one R is at position n+1;
   each R is the same or different and is independently alkyl, cycloalkyl, aminoalkyl, aralkyl, alkoxy, alkoxyalkyl, alkenyl, cycloalkenyl, alkynyl, cycloalkynyl, acyl, acylalkyl, acyloxy, acyloxyalkyl, heterocycle, aryl, heteroaryl, heteroaralkyl, heteroaralkyloxy, aroyl, aroylalkyl, aryloxy, aryloxyalkyl, halogen, haloalkyl, cyano, cyanoalkyl, nitro, nitroalkyl, carboxyl, carboxylalkyl, amino, aminoalkyl, aminocarbonyl, aminocarbonylalkyl, carbamoylalkyl, carbamoylalkoxy, iminoalkyl, imidoalkyl, alkoxycarbonyl, alkoxycarbonylalkyl, alkylamino, alkylaminoalkyl, dialkylamino, dialkylaminoalkyl, arylamino, arylaminoalkyl, hydroxy, hydroxyalkyl, isocyano, isocyanoalkyl, isothiocyano, isothiocyanoalkyl, oximinoalkoxy, morpholino, morpholinoalkyl, azido, azidoalkyl, formyl, formylalkyl, alkylthio, alkylthioalkyl, alkylsulfinyl, alkylsulfinylalkyl, alkylsulfonyl, alkylsulfonylalkyl, aminosulfonyl, arylsulfonyl, N-alkyl-N-arylaminosulfonyl, heteroatom, or heteroatom-containing group, and wherein each is optionally substituted by one or more substituents; and
R5 and R6 are the same or different and are independently alkyl, cycloalkyl, aminoalkyl, aralkyl, alkoxy, alkoxyalkyl, alkenyl, cycloalkenyl, alkynyl, cycloalkynyl, acyl, acylalkyl, acyloxy, acyloxyalkyl, heterocycle, aryl, heteroaryl, heteroaralkyl, heteroaralkyloxy, aroyl, aroylalkyl, aryloxy, aryloxyalkyl, hydrogen, halogen, haloalkyl, cyano, cyanoalkyl, nitro, nitroalkyl, carboxyl, carboxylalkyl, amino, aminoalkyl, aminocarbonyl, aminocarbonylalkyl, carbamoylalkyl, carbamoylalkoxy, iminoalkyl, imidoalkyl, alkoxycarbonyl, alkoxycarbonylalkyl, alkylamino, alkylaminoalkyl, dialkylamino, dialkylaminoalkyl, arylamino, arylaminoalkyl, hydroxy, hydroxyalkyl, isocyano, isocyanoalkyl, isothiocyano, isothiocyanoalkyl, oximinoalkoxy, morpholino, morpholinoalkyl, azido, azidoalkyl, formyl, formylalkyl, alkylthio, alkylthioalkyl, alkylsulfinyl, alkylsulfinylalkyl, alkylsulfonyl, alkylsulfonylalkyl, aminosulfonyl, arylsulfonyl, N-alkyl-N-arylaminosulfonyl, heteroatom, or heteroatom-containing group, and wherein each is optionally substituted by one or more substituents.

In embodiments, the novel compounds have the following formula:

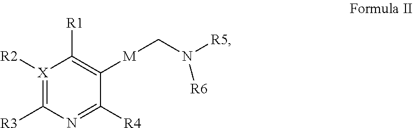

wherein,
R1 is halogen, alkyl, aryl, aralkyl, heteroaryl, alkenyl, alkynyl, cycloalkyl, heterocycle, or heteroatom-containing group, wherein the alkyl, aryl, aralkyl, heteroaryl, alkenyl, alkynyl, cycloalkyl, heterocycle, or heteroatom-containing group is optionally substituted by one or more substituents;
R2, R3, and R4 are the same or different and are independently halogen, hydrogen, alkyl, aryl, aralkyl, heteroaryl, alkenyl, alkynyl, cycloalkyl, heterocycle, or heteroatom-containing group, wherein the alkyl, aryl, aralkyl, heteroaryl, alkenyl, alkynyl, cycloalkyl, heterocycle, or heteroatom-containing group is optionally substituted by one or more substituents;
X is N or C;
M is independently

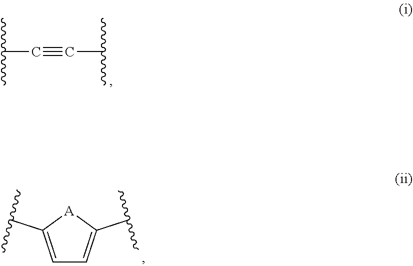

wherein A is N, O or S, or

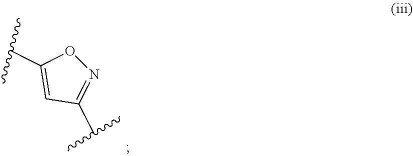 [SMILES: *c1cc(*)on1]

and
R5 and R6 are the same or different and are independently hydrogen, halogen, alkyl, aryl, aralkyl, heteroaryl, alkenyl, alkynyl, cycloalkyl, heterocycle, heteroatom-containing group, or protecting group, wherein alkyl, aryl, aralkyl, heteroaryl, alkenyl, alkynyl, cycloalkyl, heterocycle, heteroatom-containing group, or protecting group is optionally substituted by one or more substituents.

The present disclosure also describes salts and solvates of the compounds described herein. Moreover, the present disclosure describes compositions including the one or more compounds described herein, one or more salts described herein, or one or more solvates described herein, and a carrier. In embodiments, the compositions are pharmaceutical compositions

Further, the present disclosure describes methods of using the pharmaceutical compositions described herein for treating, preventing, or reducing the risk of diseases and disorders in a subject in need thereof. Examples of diseases or disorders include nicotine addiction, cancer, neurodegenerative disease, a psychiatric disorder, attention-deficit disorder (ADD), anxiety, or alcoholism.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows general scheme for the synthesis of the compounds described herein. Compound T is 5i, and Compound U is 6i.

FIGS. 2A, 2B, and 2C show exemplary compounds described herein.

FIGS. 3A and 3B show levels of nicotine or cotinine after p.o. (per os) administration.

FIGS. 3C and 3D show levels of nicotine or cotinine after i.p. (intraperitoneal) administration.

FIGS. 4A, 4B, 4C, 4D, 4E, and 4F show pharmacokinetic data.

FIG. 5 shows nicotine metabolite ratio (NMR) for Compound U-treated mice and control mice.

### DETAILED DESCRIPTION

The following are definitions of terms that may be used in the present specification. The initial definition provided for a group or term herein applies to that group or term throughout the present specification individually or as part of another group, unless otherwise indicated.

Additionally, it will be understood that any list of such candidates or alternatives is merely illustrative, not limiting, unless implicitly or explicitly understood or stated otherwise.

The terms “a,” “an,” “the” and similar referents used in the context of describing the subject matter of the present disclosure including the claims, are to be construed to cover both the singular and the plural, unless otherwise indicated herein or clearly contradicted by context.

The term “analog” (also “structural analog” or “chemical analog”) is used to refer to a compound that is structurally similar to another compound but differs with respect to a certain component, such as an atom, a functional group, or a substructure.

The term “derivative” in chemistry refers to a compound that is obtained from a similar compound or a precursor compound by a chemical reaction.

The term “nucleophile,” by itself means a chemical species that donates an electron-pair to an electrophile to form a chemical bond in a reaction. Because nucleophiles donate electrons, they are by definition Lewis bases. All molecules or ions with a free pair of electrons can act as nucleophiles.

The term “halogen” refers to a fluorine, chlorine, bromine or iodine atom.

The term “amino” refers to —NRR1, wherein R and R1 are independently, for example, hydrogen, alkyl, cycloalkyl, cycloalkenyl, cycloalkynyl, cyclic aromatic, or are joined together to give a 3 to 8-membered ring such as pyrrolidine or piperidine rings, which are optionally substituted.

The term “alkylamino” includes an amino group substituted with one alkyl group.

The term “dialkylamino” includes amino groups substituted with two groups such as —NRR₁ where R and R₁ are independently alkyl groups or together form the rest of ring such as morpholino. Examples of dialkylamino groups include dimethylamino, diethylamino and morpholino. The term “morpholinoalkyl” refers to alkyl R substituted with morpholine group.

The term “alkyl” refers to a linear or branched, saturated hydrocarbon chain including 1 to 20 carbon atoms. A lower alkyl group is an alkyl group including 1 to 6 carbon atoms.

The term “cycloalkyl” refers to a cyclic saturated hydrocarbon chain including 3 to 7 carbon ring members and including polycyclics such as bicyclics and spirocyclics.

The term “alkenyl” refers to a linear or branched unsaturated hydrocarbon chain including 2 to 20 carbon atoms and including one or more double bonds.

The term “cycloalkenyl” refers to monocyclic unsaturated hydrocarbon group including 3 to 9 carbon ring members and at least one carbon-carbon double bond.

The term “alkynyl” refers to a linear or branched unsaturated hydrocarbon chain including 2 to 20 carbon atoms and including one or more carbon-carbon triple bonds.

The term “cycloalkynyl” refers to a monocyclic unsaturated hydrocarbon chain including 3 to 9 carbon ring members and at least one triple bond.

The term “alkoxy” refers to an —OR group, wherein R is a substituent, for example, alkyl, alkenyl, alkynyl, or aralkyl.

The term “alkoxyalkyl” refers to alkyl groups having one or more alkoxy groups attached to the alkyl group. Haloalkoxy groups may contain 1 to 20 carbons.

The term “alkenyloxy” refers to —OR, wherein R is a substituent, for example, alkenyl.

The term “alkylthio” refers to —SR, wherein R is a substituent, for example, alkyl, alkenyl, alkynyl, aryl, or aralkyl.

The term “alkylthioalkyl” refers to an alkylthio group attached to an alkyl group of 1 to 20 carbon atoms through a divalent sulfur atom.

The term “alkylsulfinyl” refers to —S(O)R, wherein R is a substituent, for example, alkyl, alkenyl, alkynyl, aryl, or aralkyl.

The term “aryl” refers to an aromatic hydrocarbon ring. An aryl can be a monocyclic, bicyclic, or polycyclic aromatic hydrocarbon ring. The aromatic hydrocarbon ring can include 4 to 7 carbon atoms. Examples of aryl groups include phenyl and naphthyl groups.

The term “aryloxy” refers to —OR, wherein R is a substituent, for example, aryl or heteroaryl.

The term “aralkyl” refers to an alkyl substituted with an aryl group.

The term “heterocycle” refers to a saturated or unsaturated, cyclic or polycyclic hydrocarbon chain including one or more heteroatoms chosen from B, N, O, S, Si, and P. The hydrocarbon chain can include 3 to 20 carbon atoms. The term “heterocycle” also includes “heteroaryls.”

The term “heteroaryl” refers to an aromatic heterocyclic group, such as a cyclic or polycyclic aromatic hydrocarbon chain, including one or more heteroatoms chosen from B, N, O, S, Si, and P. Accordingly, a heteroaryl group is an example of a heterocyclic group. The aromatic hydrocarbon chain can include 3 to 20 carbons and/or heteroatoms and one or more double bonds. The polycyclic aromatic hydrocarbon chain includes two or more fused aromatic rings.

The term “heteroatom” includes any atom other than the carbon atom. Examples of heteroatoms include boron, nitrogen, oxygen, silicon, phosphorus, and sulfur.

The term “heteroatom-containing group” is a group of atoms, for example a radical, containing a heteroatom. Examples of heteroatom-containing group include —OH, —NO, —NH₂, —SH, —SOH, —SO, —SO₂, —OR, —OC(O)R, —OC(O)OH, —OC(O)OR, —OC(O)NH2, —OC(O)—NHR1, —OC(O)NR1R2, NH₂, NHR, NR1R2, —NC(O)R, —NC(O)OR, —NC(O)NH₂, —NC(O)NHR, —NC(O)NR1R2, —NC(O)SH, —NC(O)SR, —N═C═S, —N═C═O, —C(N)NR1R2, —C(N)R, —S(O)OH, —S(O)OR, —S(O)₂OH, —S—OR, or —S(O)₂OR, wherein R, R1, and R2 are substituents, and are same or different and independently, for example, hydrogen, alkyl, alkyl, cycloalkyl, aralkyl, alkenyl, alkynyl, heterocycle, aryl, or heteroaryl.

The term “sulfonyl” refers to —S(O)₂—R, wherein R is a substituent, for example alkyl, alkenyl, alkynyl, aryl, aralkyl.

The term “aminosulfonyl”, “sulfamyl”, “sulfonamidyl” refer to —SO₂NRR1, wherein R and R1 are substituents and independently selected from alkyl, alkenyl, alkynyl, aryl, aralkyl.

The term “hydroxyalkyl” refers to linear or branched alkyl groups having 1 to 20 carbon atoms any one of which may be substituted with a hydroxyl group.

The term “cyanoalkyl” refers to linear or branched alkyl groups having 1 to 20 carbon atoms any one of which could be substituted with one or more cyano (—CN) groups.

The term “oximinoalkoxy” refers to alkoxy groups having 1 to 20 carbon atoms, any one of which may be substituted with an oximino (—C(N)—OR) group.

The term “aroyl” refers to —C(O)R, wherein R is aryl group.

The term “alkoxycarbonyl” refers to —C(O)OR, wherein R is a substituent, for example, alkyl, alkenyl, alkynyl, aryl, or aralkyl.

The term “acyl” refers to the alkanoyl group —C(O)R, wherein R is a substituent, for example, alkyl, alkenyl, alkynyl, aryl, or aralkyl.

The term “acyloxy” refers to the alkanoyl group —OC(O)R, wherein R is a substituent, for example, alkyl, alkenyl, alkynyl, aryl, or aralkyl.

The term “aminoalkyl” refers to alkyl which is substituted with amino groups. The amino groups can be further substituted.

The term “arylamino” refers to amino groups substituted with one or more aryl groups.

The term “aminocarbonyl” refers to —C(O)NRR₁, wherein R and R1 are substituents and are same or different and independently selected from hydrogen, alkyl, alkenyl, alkynyl, aryl, or aralkyl.

The azidoalkyl refers to alkyl which is substituted with an azido (—N₃) group.

The term “isocyanoalkyl” refers to alkyl that is substituted with isocyano group —NCO.

The term “isothiocyanoalkyl” refers to alkyl R that is substituted with isothiocyano group —NCS.

The term “isocyanoalkenyl” refers to alkenyl R that is substituted with isocyano group —NCO.

The term “formylalkyl” refers to alkyl R substituted with —CHO.

The term “linker” refers to a bond or a functional group that connects a first atom to a second atom. The first and second atoms may be connected to other atoms. The bond can be a direct single bond between the two atoms. The bond can also be a double bond or triple bond. The functional group can be any substituent capable of linking the two atoms.

The term “ring system” or “ring structure” refers to an organic cyclic compound in which an organic compound containing a series of atoms is connected to form a loop or ring. The term includes various cyclic compounds, which may be: saturated, unsaturated or aromatic; substituted or unsubstituted; hetero- or homo- or spirocyclic; and may be mono- or polycyclic, as described herein. As used herein, hetero-structures are those in which not all atoms of the primary structure are carbon. Instead, one or more are a different atom, for example, a 6-membered ring in which 5 of the ring atoms are C and one is N.

The term “substituent” can be an atom or a group of atoms. For example, a substituent can be halogen, hydroxyl, amino, amide, cyano, nitro, alkyl, alkoxy, alkenyl, alkynyl, mercapto, carboxyl, aryl, heterocycle, sulfonyl, alkylthio, alkylsulfinyl, alkylsulfonyl, alkanoyl, alkanoyloxy, alkanoyloxyalkanoyl, alkoxycarboxy, aminocarbonyl, azido, keto, alkanoylamido, heteroaryloxy, carbamyl, heterocarbocyclicoxy, and any group described herein.

The term “optionally substituted” or “substituted” refers to further substituting the group of atoms (or radicals) described herein (including the heteroatom-containing groups) with one or more substituents, where appropriate. For example, an alkyl can be unsubstituted or optionally substituted. However, a halogen cannot be substituted.

“Metal” or “metal ion” includes a soluble form of a transition metal or s- or f-block metal in an oxidation state that is known in the art.

The terms “treating,” “treatment,” or “therapy” of a disease or disorder means slowing, stopping, or reversing progression of the disease or disorder, as evidenced by a reduction or elimination of either clinical or diagnostic symptoms, using the compositions and methods of the present invention as described herein.

The terms “preventing,” “prophylaxis,” or “prevention” of a disease or disorder means prevention of the occurrence or onset of a disease or disorder or some or all of its symptoms.

The terms “parenteral carrier system” (including variations thereof such as the various specific injectable and infusible dosage forms) refer to compositions comprising one or more pharmaceutically suitable excipients, such as solvents like water and co-solvents, solubilizing compounds, wetting compounds, suspending compounds, thickening compounds, emulsifying compounds, chelating compounds, buffers, pH adjusters, antioxidants, reducing compounds, antimicrobial preservatives, bulking compounds, protectants, tonicity adjusters and special additives.

The term “dose-concentrate” refers to a pharmaceutical composition comprising a provided formulation, wherein the concentration of active agent(s) is higher than a typical unit dosage form concentration administered directly to a subject. A dose-concentrate may be used as provided for administration to a subject, but is generally further diluted to a typical unit dosage form concentration in preparation for administration to a subject. The entire volume of a dose-concentrate, or aliquots thereof, may be used in preparing unit dosage form(s) for treatment, for example, by the methods provided herein. In some embodiments, a dose-concentrate is about 2 fold, about 5-fold, about 10-fold, about 25-fold, about 50-fold, about 100-fold, or about 200-fold more concentrated than a unit dosage form. In certain embodiments, a dose concentrate is about 50-fold, about 100-fold, or about 200-fold more concentrated than a unit dosage form.

As used herein, an “effective amount” or a “therapeutically effective amount” of a compound or pharmaceutically acceptable formulation can achieve a desired therapeutic and/or prophylactic effect. In embodiments, an “effective amount” is at least a minimal amount of a compound, or formulation containing a compound, which is sufficient for treating one or more symptoms of a disorder or condition associated with modulation of peripheral p opioid receptors, such as side effects associated with opioid analgesic therapy (e.g., gastrointestinal dysfunction (e.g., dysmotility constipation, etc.), nausea, emesis, (e.g., vomiting), etc.). In embodiments, an “effective amount” of a compound, or formulation containing a compound, is sufficient for treating symptoms associated with, a disease associated with aberrant endogenous peripheral opioid or p opioid receptor activity (e.g., idiopathic constipation, ileus, etc.).

The term “formulation” refers to a composition that includes at least one pharmaceutically active compound (e.g., at least methylnaltrexone) in combination with one or more excipients or other pharmaceutical additives for administration to a subject. In general, particular excipients and/or other pharmaceutical additives are typically selected with the aim of enabling a desired stability, release, distribution and/or activity of active compound(s) for applications.

The terms “therapeutically effective dose” or “therapeutically effective amount” refer to an amount, dose or dosing regimen of a compound (i.e., active pharmaceutical ingredient, prodrug or precursor thereof) that, upon interaction with a biological material, is sufficient to treat or prevent injury or undesirable conditions, whereby such dose may vary depending on the form of the compound, the biological material's condition and/or severity, the route of administration, the age of the biological material and the like.

In recent years, studies have been focused on inhibition of CYP2A6, which is involved in nicotine metabolism, as a strategy of treating nicotine addiction because smokers adjust their tobacco use to maintain a certain blood brain level of nicotine. Inhibition has been shown to result in altered pharmacokinetics for nicotine resulting in an increased plasma half-life. Nicotine is metabolized to cotinine. UGT2B10 catalyzes the N-glucuronidation of both nicotine and cotinine. Accordingly, inhibition of nicotine metabolism should result in a diminished desire to smoke and a lessening of the ingestion toxic or carcinogenic components of smoke.

The present disclosure describes novel compounds that inhibit CYP2A6 and/or UGT2B10 mediated nicotine metabolism. Such inhibitors include compounds of

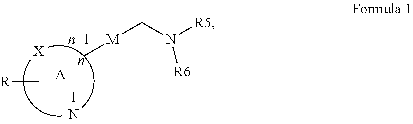

wherein,
A is a heterocycle comprising X, N, and one to 5 carbon atoms, and N is at position 1 on the heterocycle;
X is C, N, O, or S, and X is at any position on the heterocycle that is not occupied by N or a carbon atom bonded to M;
M a linker, and M is bonded to the carbon atom on the heterocycle at position n, wherein n is 2, 3, 4, 5, or 6;
R is one or more substituents on A, and at least one R is at position n+1;
   each R is the same or different and is independently alkyl, cycloalkyl, aminoalkyl, aralkyl, alkoxy, alkoxyalkyl, alkenyl, cycloalkenyl, alkynyl, cycloalkynyl, acyl, acylalkyl, acyloxy, acyloxyalkyl, heterocycle, aryl, heteroaryl, heteroaralkyl, heteroaralkyloxy, aroyl, aroylalkyl, aryloxy, aryloxyalkyl, halogen, haloalkyl, cyano, cyanoalkyl, nitro, nitroalkyl, carboxyl, carboxylalkyl, amino, aminoalkyl, aminocarbonyl, aminocarbonylalkyl, carbamoylalkyl, carbamoylalkoxy, iminoalkyl, imidoalkyl, alkoxycarbonyl, alkoxycarbonylalkyl, alkylamino, alkylaminoalkyl, dialkylamino, dialkylaminoalkyl, arylamino, arylaminoalkyl, hydroxy, hydroxyalkyl, isocyano, isocyanoalkyl, isothiocyano, isothiocyanoalkyl, oximinoalkoxy, morpholino, morpholinoalkyl, azido, azidoalkyl, formyl, formylalkyl, alkylthio, alkylthioalkyl, alkylsulfinyl, alkylsulfinylalkyl, alkylsulfonyl, alkylsulfonylalkyl, aminosulfonyl, arylsulfonyl, N-alkyl-N-arylaminosulfonyl, heteroatom, or heteroatom-containing group, and wherein each is optionally substituted by one or more substituents; and
R5 and R6 are the same or different and are independently alkyl, cycloalkyl, aminoalkyl, aralkyl, alkoxy, alkoxyalkyl, alkenyl, alkynyl, acyl, acylalkyl, acyloxy, acyloxyalkyl, heterocycle, aryl, heteroaryl, heteroaralkyl, heteroaralkyloxy, aroyl, aroylalkyl, aryloxy, aryloxyalkyl, hydrogen, halogen, haloalkyl, cyano, cyanoalkyl, nitro, nitroalkyl, carboxyl, carboxylalkyl, amino, aminoalkyl, aminocarbonyl, aminocarbonylalkyl, carbamoylalkyl, carbamoylalkoxy, iminoalkyl, imidoalkyl, alkoxycarbonyl, alkoxycarbonylalkyl, alkylamino, alkylaminoalkyl, dialkylamino, dialkylaminoalkyl, arylamino, arylaminoalkyl, hydroxy, hydroxyalkyl, isocyano, isocyanoalkyl, isothiocyano, isothiocyanoalkyl, oximinoalkoxy, morpholino, morpholinoalkyl, azido, azidoalkyl, formyl, formylalkyl, alkylthio, alkylthioalkyl, alkylsulfinyl, alkylsulfinylalkyl, alkylsulfonyl, alkylsulfonylalkyl, aminosulfonyl, arylsulfonyl, N-alkyl-N-arylaminosulfonyl, heteroatom, or heteroatom-containing group, and wherein each is optionally substituted by one or more substituents.

In embodiments, the compound of Formula I has is a 5- or 6-membered heterocyclic ring system (A), such as pyrazole, imidazole, isoxazole, oxazole, isothiazole, thiazole, pyridine, pyrimidine, pyridazine, pyrazine, thiophene, and furan.

In embodiments, the linker, M, of the compound of Formula I is a direct bond or any functional group connecting the A to the methylene group. In embodiments, M is a direct single bond, a carbon-carbon single bond,

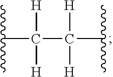 [SMILES: *CC*]

a carbon-carbon double bond,

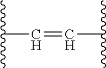 [SMILES: *C=C*]

a carbon-carbon triple bond;

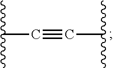 [SMILES: *C#C*]

or a thiophene,

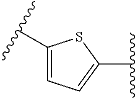 [SMILES: *c1ccc(*)s1]

a furan,

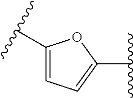 [SMILES: *c1ccc(*)o1]

or alternatively a 1,3-disubstituted 5-membered heterocycle or carbocycle.

In embodiments, the novel compounds have the following formula:

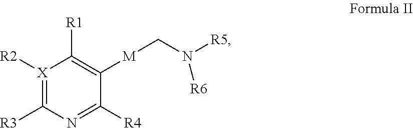

wherein,
R1 is halogen, alkyl, aryl, aralkyl, heteroaryl, alkenyl, alkynyl, cycloalkyl, heterocycle, or heteroatom-containing group, wherein the alkyl, aryl, aralkyl, heteroaryl, alkenyl, alkynyl, cycloalkyl, heterocycle, or heteroatom-containing group is optionally substituted by one or more substituents;
R2, R3, and R4 are the same or different and are independently halogen, hydrogen, alkyl, aryl, aralkyl, heteroaryl, alkenyl, alkynyl, cycloalkyl, heterocycle, or heteroatom-containing group, wherein the alkyl, aryl, aralkyl, heteroaryl, alkenyl, alkynyl, cycloalkyl, heterocycle, or heterocycle-containing group is optionally substituted by one or more substituents;
X is N or C;
M is independently

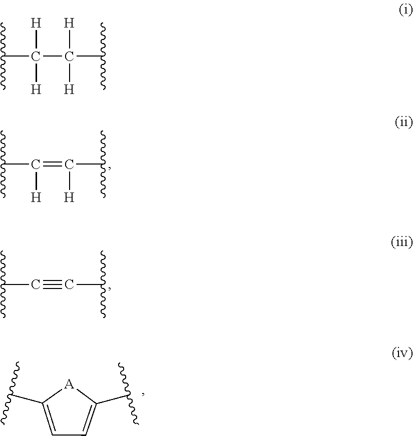

wherein A is N, O, or S, or

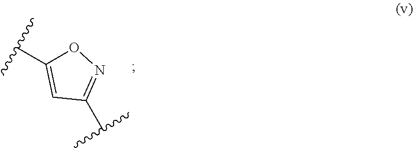 [SMILES: *c1cc(*)on1]

(vi) direct bond; and
R5 and R6 are the same or different and are independently hydrogen, halogen, alkyl, aryl, aralkyl, heteroaryl, alkenyl, alkynyl, cycloalkyl, heterocycle, heteroatom-containing group, or protecting group, wherein alkyl, aryl, aralkyl, heteroaryl, alkenyl, alkynyl, cycloalkyl, heterocycle, heteroatom-containing group, or protecting group is optionally substituted by one or more substituents.

In embodiments, the compound described herein of Formula II has a linker M, wherein M is a direct bond,

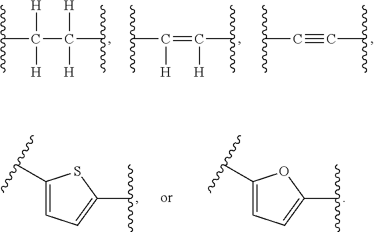

The compounds described herein are also referred to nicotine analogues.

Representative examples of the compounds described herein include:
Compound A: 3-(isoquinolin-4-yl)prop-2-yn-1-amine dihydrochloride;
Compound B: 3-(quinolin-3-yl)prop-2-yn-1-amine;
Compound C: 3-(4-methylpyridin-3-yl)prop-2-yn-1-amine;
Compound D: 3-(5-methylpyridin-3-yl)prop-2-yn-1-amine;
Compound E: 3-(6-methylpyridin-3-yl)prop-2-yn-1-amine;
Compound F: 3-(2-methylpyridin-3-yl)prop-2-yn-1-amine;
Compound G: 3-(5-methylpyridin-3-yl)prop-2-yn-1-amine;
Compound H: 3-(4-ethylpyridin-3-yl)prop-2-yn-1-amine;
Compound I: 3-(4-propylpyridin-3-yl)prop-2-yn-1-amine;
Compound J: 3-(4-phenylpyridin-3-yl)prop-2-yn-1-amine;
Compound K: 3-(4-methoxypyridin-3-yl)prop-2-yn-1-amine;
Compound L: 3-(4-chloropyridin-3-yl)prop-2-yn-1-amine dihydrochloride;
Compound M: 3-(4-Furan-2-yl-pyridin-3-yl)-prop-2-ynylamine dihydrochloride;
Compound N: 3-(4-Furan-3-yl-pyridin-3-yl)-prop-2-ynylamine dihydrochloride;
Compound O: 3-([3,4′-Bipyridin]-3′-yl)prop-2-yn-1-amine trihydrochloride;
Compound P: 3-([4,4′-Bipyridin]-3′-yl)prop-2-yn-1-amine trihydrochloride;
Compound Q: 3-(4-(Pyrimidin-5-yl)pyridin-3-yl)prop-2-yn-1-amine trihydrochloride;
Compound R: (5-(4-methylpyridin-3-yl)furan-2-yl)methanamine;
Compound S: (5-(4-methylpyridin-3-yl)thiophene-2-yl)methanamine;
Compound T: (5-(4-ethylpyridin-3-yl)furan-2-yl)methanamine dihydrochloride;
Compound U: (5-(4-ethylpyridin-3-yl)thiophene-2-yl)methanamine dihydrochloride;
Compound V: (5-(4-propylpyridin-3-yl)furan-2-yl)methanamine dihydrochloride;
Compound W: (5-(4-propylpyridin-3-yl)thiophene-2-yl)methanamine dihydrochloride;
Compound X: (5-(4-phenylpyridin-3-yl)furan-2-yl)methanamine;
Compound Y: (5-(4-phenylpyridin-3-yl)thiophene-2-yl)methanamine;
Compound Z: (5-(4-methoxypyridin-3-yl)furan-2-yl)methanamine dihydrochloride;
Compound AA: (5-(4-methoxypyridin-3-yl)thiophene-2-yl)methanamine dihydrochloride;
Compound AB: (5-(4-chloropyridin-3-yl)thiophene-2-yl)methanamine dihydrochloride;
Compound AC: (5-(4-(furan-2-yl)pyridin-3-yl)furan-2-yl)methanamine dihydrochloride;
Compound AD: (5-(4-(furan-2-yl)pyridin-3-yl)thiophene-2-yl)methanamine dihydrochloride;
Compound AE: (5-(4-(furan-3-yl)pyridin-3-yl)furan-2-yl)methanamine dihydrochloride;
Compound AF: (5-(4-(furan-3-yl)pyridin-3-yl)thiophene-2-yl)methanamine dihydrochloride;
Compound AG: (5-([3,4′-Bipyridin]-3′-yl)furan-2-yl)methanamine trihydrochloride;
Compound AH: (5-([3,4′-Bipyridin]-3′-yl)thiophene-2-yl)methanamine trihydrochloride;
Compound AI: (5-([4,4′-Bipyridin]-3′-yl)furan-2-yl)methanamine trihydrochloride;
Compound AJ: (5-([4,4′-Bipyridin]-3′-yl)thiophene-2-yl)methanamine trihydrochloride;
Compound AK: (5-(4-(Pyrimidin-5-yl)pyridin-3-yl)furan-2-yl)methanamine trihydrochloride; and
Compound AL: (5-(4-(Pyrimidin-5-yl)pyridin-3-yl)thiophene-2-yl)methanamine trihydrochloride;

The present disclosure also describes analogues and derivatives of the compounds described herein. The analogues and derivatives described herein are able to block CYP2A6 mediated nicotine metabolism.

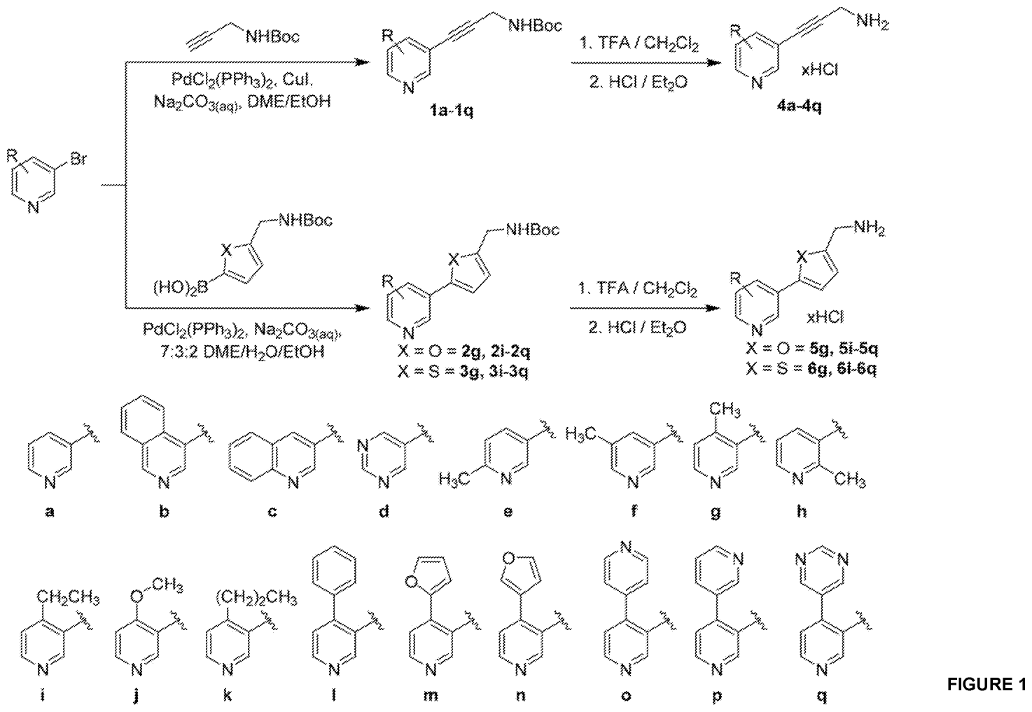

FIG. 1 provides a general scheme for synthesizing the compounds described herein. The Examples below provide detailed process for the synthesis of each of the representative compounds.

Various protecting groups are used in the synthesis of the compounds described herein. Examples of protecting groups for the nitrogen atom include t-butyloxycarbonyl (t-Boc), carbobenzyloxy (CBZ), 9-fluorenylmethyloxycarbonyl (FMOC), acetyl, trifluoroacetyl, benzyl, dibenzyl, trityl and p-toluenesulfonyl.

The present disclosure also describes salts and solvates of the compounds, derivatives, and analogues described herein and solvates of the salts described herein. The compounds described herein may form salts: with alkali metals such as sodium, potassium and lithium; with alkaline earth metals such as calcium and magnesium; with organic bases such as dicyclohexylamine, tributylamine, pyridine; and with amino acids such as arginine, lysine and the like. Moreover, the compounds described herein may form salts with a variety of organic and inorganic acids. Such salts include those formed with hydrogen chloride, hydrogen bromide, methanesulfonic acid, sulfuric acid, acetic acid, trifluoroacetic acid, oxalic acid, maleic acid, benzenesulfonic acid, toluenesulfonic acid and various others (e.g., nitrates, phosphates, borates, tartrates, citrates, succinates, benzoates, ascorbates, salicylates and the like). The formation of such salts is well-known to those skilled in the art.

Additionally, the present disclosure describes pharmaceutically acceptable salts and solvates described herein. Pharmaceutically acceptable (i.e., non-toxic, physiologically acceptable) salts are used in the treatment of a subject. Other salts are useful, for example in isolating or purifying the compounds of this invention.

Moreover, the present disclosure describes compositions including one or more compounds described herein, one or more salts described herein, or one or more solvates described herein, and a carrier. In embodiments, the compositions are pharmaceutical compositions including one or more compounds, derivatives, analogues, pharmaceutically acceptable salts, or pharmaceutically acceptable solvates described herein and a pharmaceutically acceptable carrier. The pharmaceutical compositions are for treating subjects in need thereof.

Further, the present disclosure describes methods of using the compounds, salts, solvates, derivatives, and analogues described herein, and the pharmaceutical compositions described herein for treating, preventing, or reducing the risk of diseases and disorders in a subject that would benefit from blocking CYP2A6 and/or UBT2B10 mediated nicotine metabolism. Examples of such diseases and disorders include tobacco addiction and complications that arise from tobacco use or addiction, for example, cancer, alcoholism, neurodegenerative disease, a psychiatric disorder, heart (cardiovascular) disease, stroke, blindness, cataracts, periodontitis, aortic aneurysm, atherosclerosis, pneumonia, chronic obstructive pulmonary disease, asthma, diabetes, reduced fertility, ectopic pregnancy, erectile dysfunction, rheumatoid arthritis, or alcoholism. Examples of cancer include oropharyngeal cancer, laryngeal cancer, esophageal cancer, tracheal cancer, bronchial cancer, lung cancer, acute myeloid leukemia, stomach cancer, liver cancer, pancreatic cancer, kidney cancer, ureter cancer, cervical cancer, bladder cancer, and colorectal cancer. Examples of psychiatric disorder include anxiety disorders such as post-traumatic stress disorder, bipolar disorder, generalized anxiety disorder, obsessive-compulsive disorder, panic disorder, separation anxiety, social anxiety disorder, and attention deficit disorder. In embodiments, the pharmaceutical compositions described herein can also be used to enhance cognition or to induce a neuroprotective effect in a subject in need thereof.

The present disclosure describes methods of treating, preventing, and/or reducing the risk of diseases or disorders in a subject by blocking CYP2A6 and/or UGT2B10 mediated nicotine metabolism. Methods described herein include treating subjects including all mammals, for example humans and research animals. Subjects in need of a treatment (in need thereof) are subjects having a disease or disorder that would benefit from blocking CYP2A6 mediated nicotine metabolism. The methods include administering the pharmaceutical composition, a compound, a salt, solvate, a derivative, or an analog described herein to a subject in need thereof.

In embodiments, the present disclosure describes methods for treating, preventing or reducing the risk of tobacco or nicotine addiction. Such methods decrease the subject's need to smoke, which also treat, prevent, or reduce the risk of complications that arise from tobacco or nicotine addiction. Moreover, the compounds described herein can be used to block or inhibit CYP2A13 activity. CYP2A13 is known to activate pro-carcinogens in tobacco smoke and other sources. CYP2A13 is an enzyme that exhibits high homology with CYP2A6 and exhibits higher expression in the human lung than CYP2A6. Both CYP2A13 and CYP2A6 activate the group of carcinogens known as tobacco-specific nitrosamines (e.g., NNK, NNN), which are derived from nicotine during the tobacco-curing process, the compounds described herein can inhibit a key carcinogen-activating pathway. In embodiments, the compounds, salts, solvates, derivatives, and analogues described herein and the pharmaceutical composition described herein can be used to treat, prevent, or reduce the risk of diseases such as cancer, including lung, esophageal, oral, laryngeal, oropharyngeal, tonsil, tongue, bladder, and pancreatic cancer. In embodiments, blocking or inhibiting CYP2A13 and CYP2A6 activation of carcinogens treats, prevents, or reduces the risk of developing a disease such as cancer.

The compounds, salts, solvates, derivatives, and analogues described herein, and the pharmaceutical compositions described herein can be formulated for administration to a subject in need thereof. They can be formulated for administration, for example, as a tablet, pill, capsule, gel, geltab, liquid, cream, lotion, aerosol, patch, or implant.

Administration of the formulation to the subject in need thereof may be carried out in any convenient manner, including by inhalation, injection, ingestion, transfusion, implantation, or transplantation. In embodiments, the formulation is administered topically, transdermally, enterally or parenterally. Methods of enteral administration includes oral, sublingual and rectal administration. Methods of parenteral administration include subcutaneous, intradermal, intramuscular, intrathecal, epidural, intravenous, intracerebral, intracranial, and intraperitoneal.

The formulations described herein may include one or more parenteral carrier systems with or without one or more diluents, emulsifiers, preservatives, buffers, or excipients. The formulations include pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, and suspensions, suitable for ingestion by the subject.

Pharmaceutical preparations for oral use may be formulated with one or more solid excipients. Examples of solid excipients include carbohydrate or protein fillers, for example, sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, such as gelatin and collagen. Disintegrating or solubilizing agents may be added, such as the cross-linked polyvinyl pyrrolidone, agar, alginic acid, or a salt thereof, such as sodium alginate.

For manufacturing liquid formulations, liquid carriers are used. Such carriers are used for preparing solutions, suspensions, emulsions, syrups, elixirs and pressurized formulations. The active ingredient (compound, derivative, analog, salt, or solvate described herein) can be dissolved or suspended in a pharmaceutically acceptable liquid carrier such as water, an organic solvent, a mixture of both or pharmaceutically acceptable oils or fats. The liquid carrier can include other suitable pharmaceutical additives such as solubilizers, emulsifiers, buffers, preservatives, sweeteners, flavoring agents, suspending agents, thickening agents, colors, viscosity regulators, stabilizers or osmo-regulators.

For manufacturing solid formulations, solid carriers are used. Examples of solid carriers include substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents. It can also be an encapsulating material. For powders, the carrier can be a finely divided solid which is in admixture with the finely divided active compound. For tablets, the active compound is mixed with a carrier having the necessary compression properties in suitable proportions and compacted in the shape and size desired. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Methods for preparing therapeutic formulations are well-known. Such methods are described by Brunton et al., eds., Goodman and Gilman's: The Pharmacological Bases of Therapeutics, 12th ed., McGraw-Hill, 2011; Remington: The Science and Practice of Pharmacy, Mack Publishing Co., 20th ed., 2000; Avis et al., eds., Pharmaceutical Dosage Forms: Parenteral Medications, published by Marcel Dekker, Inc., N.Y., 1993; Lieberman et al., eds., Pharmaceutical Dosage Forms: Tablets, published by Marcel Dekker, Inc., N.Y., 1990; and Lieberman et al., eds., Pharmaceutical Dosage Forms: Disperse Systems, published by Marcel Dekker, Inc., N.Y. 1990.

In embodiments, concentrations of the active compound in a formulation or pharmaceutical composition can be varied such that 10-200 mg/kg will be delivered in 1-4 unit formulations to an adult.

The effective dosage schedule and amounts will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

In embodiments, the methods described herein are to treat subjects of nicotine addiction or tobacco use by delaying the onset of withdrawal symptoms typically experienced by tobacco users, thus delaying subsequent tobacco use and decreasing behavioral addiction. In embodiments, the formulation described herein is administered by itself in an effective amount to the subject in need thereof. The formulation described herein can also be administered in combination with another therapy for effective treatment.

The methods described herein also includes combination therapy including the formulations described herein and another therapy. In embodiments, the formulations described herein is used with an alternative nicotine delivery system such as a nicotine patch, a nicotine lozenge, nicotine gum, nicotine nasal spray or nicotine inhaler. In embodiments, the formulations or compositions described herein is used in combination with second active agent for treating or preventing the disease or disorder. As an example, the second active agent can be an antidepressant, a selective nicotinic receptor antagonist, a nicotinic receptor partial agonist, or a cholinesterase inhibitor.

Moreover, the methods described herein includes blocking CYP2A6- and/or UGT2B10-mediated nicotine metabolism in cells in vivo or in vitro. The methods include administering the compound, salt, solvate, analogues, or derivatives described herein or the pharmaceutical composition described herein to cells overexpressing CYP2A6- and/or UGT2B10-mediated nicotine metabolism and assaying for nicotine metabolites. Examples include liver microsomes, specifically human or mouse liver microsomes, as well as CYP- or UGT-over-expressing cell microsomes. Examples of nicotine metabolites include cotinine, 3-OH-cotinine, and nicotine-N′-oxide.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

In addition, unless otherwise indicated, numbers expressing quantities of ingredients, constituents, reaction conditions and so forth used in the specification and claims are to be understood as being modified by the term “about.” Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±15% of the stated value; ±10% of the stated value; ±5% of the stated value; ±4% of the stated value; ±3% of the stated value; ±2% of the stated value; ±1% of the stated value; or ±any percentage between 1% and 20% of the stated value.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the described subject matter.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

The following examples illustrate exemplary methods provided herein. These examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### EXAMPLES

### Example 1. Chemical Synthesis

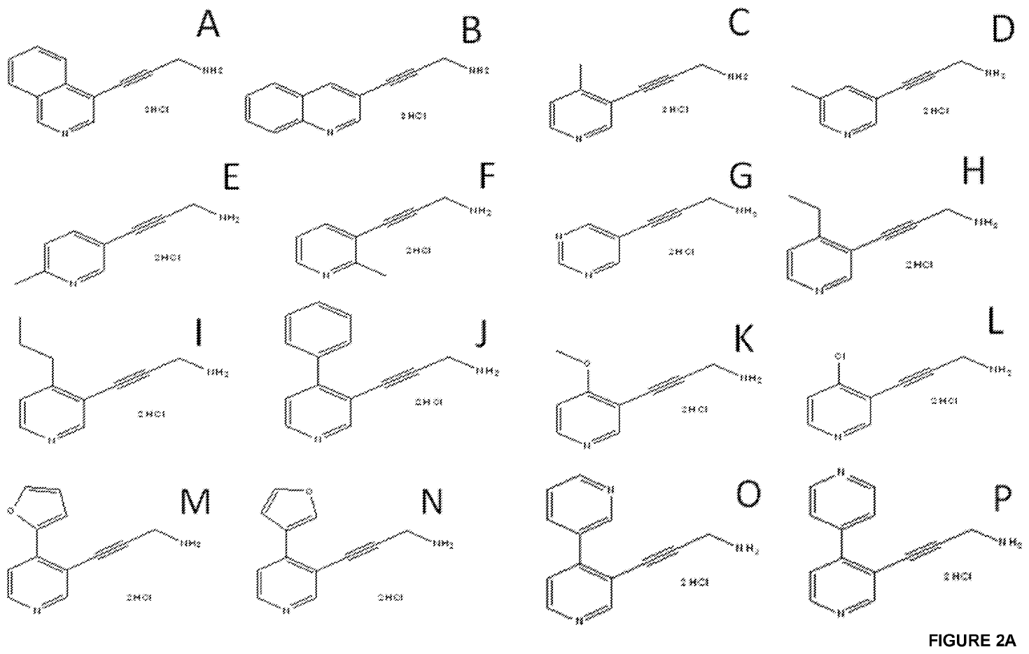

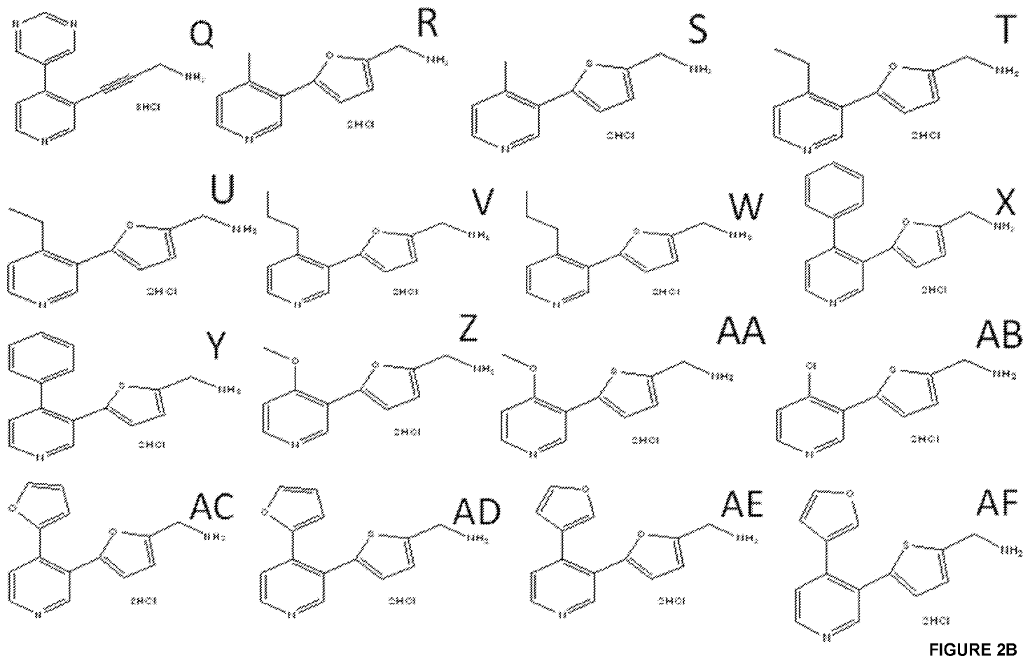

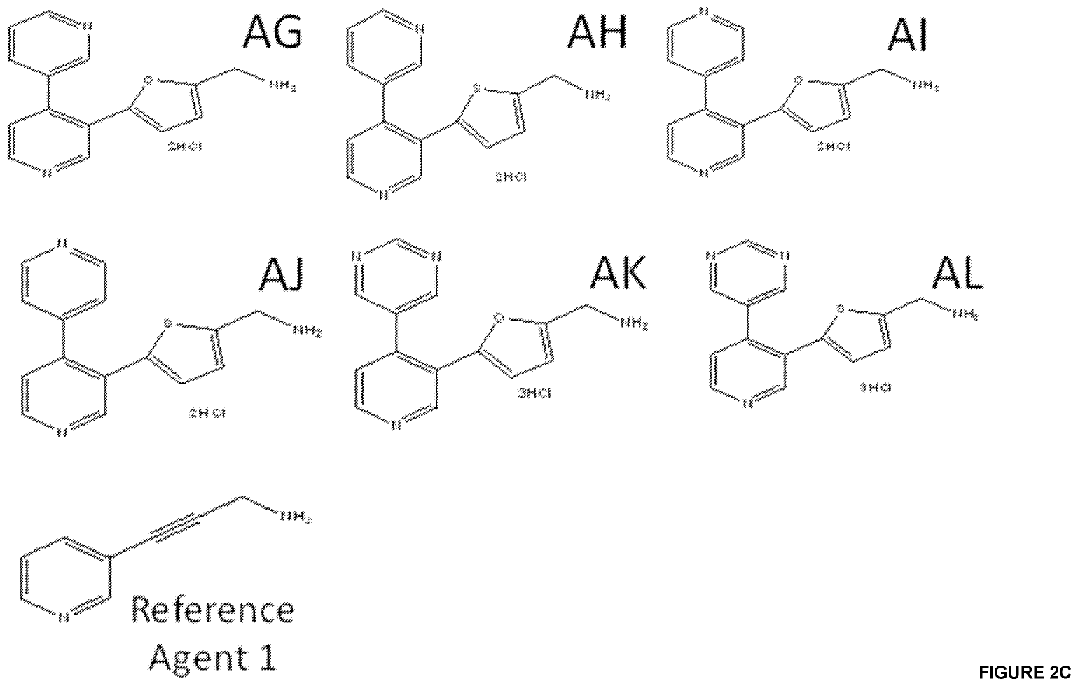

Introduction: A series of novel compounds were synthesized. The activity and specificity of a number of isoquinoline-, quinoline- and pyridine-based agents as inhibitors of enzymes involved in the metabolism of nicotine, using human liver microsomes as an ex vivo model were evaluated. The agents shown in FIG. 2 include the reference agent utilized as a lead compound and new compositions of matter (A through AL) that where synthesized and tested for inhibitory effects against CYP2A6 as well as eight major hepatic CYPs. Several of these agents were also tested for their inhibitory activity against UGT2B10, which plays a major role in the glucuronidation of nicotine, which accounts for 3-7% of total nicotine metabolism. Aryl halides were cross-coupled with suitably protected alkynes or aryl boronic acids to make the protected agents (N—BOC Agents), which were subsequently deprotected by treatment with trifluoroacetic acid in dichloromethane followed by dihydrochloride salt formation using anhydrous hydrogen chloride in ethyl ether. All intermediates were characterized and analyzed for purity by ¹H NMR, and all final compounds were characterized and analyzed for purity by ¹H and ¹³C NMR on a Bruker AVANCE 300 or 500 MHz NMR and ultra performance liquid chromatography (UPLC) and composition by high resolution mass spectrometry (HRMS). UPLC and HRMS were measured on a Waters Acquity UPLC coupled to a Xevo G2-S QT of mass spectrometer. For purity analysis, λ was 254 nm with retention times (t_{R}) evaluated in minutes; for HRMS, the mass spectrometer was employed. ¹H NMR, ¹³C NMR and mass spectra were consistent with the assigned structures.

### Compound A-Boc: tert-butyl (3-(isoquinolin-4-yl)prop-2-yn-1-yl)carbamate

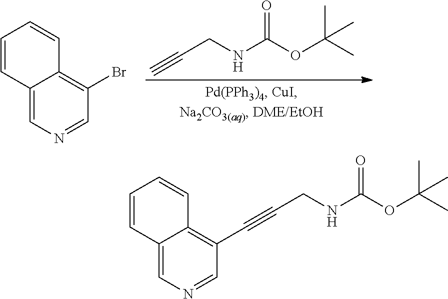

To a 100 mL round bottom flask containing N-Boc-propargylamine (902. mg, 5.80 mmol) under a blanket of argon_{(g)} was added 40 mL of degassed 1-propanol followed by tetrakis(triphenylphosphine)palladium(0) (268. mg, 0.230 mmol) by cuprous iodide (110. mg, 0.580 mmol). To the vigorously stirred suspension was added sodium carbonate (801. mg, 7.55 mmol) dissolved in degassed water (ca. 2.0 mL). The flask was purged with argon_{(g)}, stirred at ambient temperature for ten minutes followed by the addition of a solution of 3-bromo-isoquinoline (1133. mg, 5.40 mmol) in 4 mL of degassed 1-propanol. The mixture was refluxed under argon_{(g)} for 24 hours. The contents of the flask were transferred to a sintered glass funnel containing anhydrous Na₂SO₄. The crude material was eluted with methanol. The solvent was removed in vacuo and the residue was chromatographed on silica gel (EtOAc/Hex, 25:75, v/v to EtOAc/Hex, 50:50, v/v, TLC: EtOAc/Hex, 50:50, R_{f}=0.44) to afford A-Boc (936 mg, 71% yield) as a red oil ¹H NMR (CDCl₃) d 9.19 (s, 1H), 8.66 (s, 1H), 8.26-8.19 (m, 2H), 8.02-7.96 (m, 1H), 7.82-7.74 (m, 1H), 7.70-7.61 (m, 1H), 4.32 (s, 2H), 1.50 (s, 9H).

### Compound A: 3-(isoquinolin-4-yl)prop-2-yn-1-amine Dihydrochloride

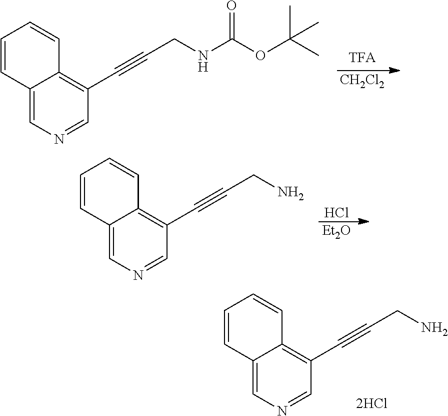

To a solution of A-Boc (56.1 mg, 0.200 mmol) in anhydrous dichloromethane (ca. 1.5 mL) was added trifluoroacetic acid (4 mL) and the resultant solution was stirred at ambient temperature for 1.5 hours. The mixture was diluted with 20 mL of water, transferred to a separatory funnel and the organics were discarded. The aqueous portion was washed with dichloromethane (2×40 mL), the pH was adjusted to ca. 10 using 10 N NaOH and extracted with dichloromethane (2×50 mL). The combined organics were dried over anhydrous MgSO₄, filtered and the solvent was removed in vacuo. This material was dissolved in 40 mL of diethyl ether and treated with 5 mL of ethereal HCl. The resulting precipitate was collected by centrifugation and dried in vacuo to afford A (58.6 mg, 68.1% yield) as a white solid: 1H NMR (300 MHz, D₂O) d 9.32 (s, 1H), 8.45 (s, 1H), 8.15-8.22 (m, 2H), 7.99-8.06 (m, 1H), 7.77-7.84 (m, 1H), 4.13 (s, 2H); 13C NMR (75 MHz, D₂O) d 147.4, 137.6, 137.2, 135.6, 131.1, 130.6, 126.6, 125.0, 118.2, 90.7, 78.7, 29.6.

### Compound B-Boc: tert-butyl (3-(quinolin-3-yl)prop-2-yn-1-yl)carbamate

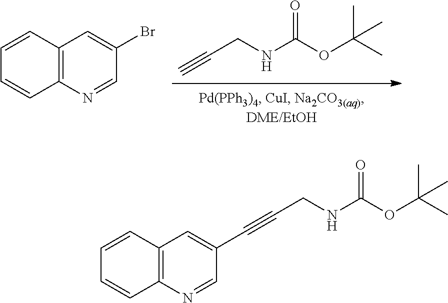 [SMILES: Brc1cnc2ccccc2c1 ; C#CCNC(=O)OC(C)(C)C ; CC(C)(C)OC(=O)NCC#Cc1cnc2ccccc2c1]

To a Biotage 2.0-5.0 mL microwave tube containing N-Boc-propargylamine (845. mg, 5.40 mmol) under a blanket of argon_{(g)} was added cuprous iodide (103. mg, 0.540 mmol) followed by tetrakis(triphenylphosphine)palladium(0) (320. mg, 0.277 mmol) followed by degassed 1-propanol (2.0 mL). To the vigorously stirred suspension was added sodium carbonate (750. mg, 7.08 mmol) dissolved in degassed water (ca. 2.0 mL). The tube was purged with argon_{(g)}, stirred at ambient temperature for ten minutes followed by the addition of a solution of 3-bromo-quinoline (1133 mg, 5.4 mmol). The tube was purged with argon_{(g)}, capped and placed in a Biotage Initiator+ microwave and heated to 100° C. for 15 minutes on normal absorption level. The contents of the flask were transferred to a sintered glass funnel containing anhydrous Na₂SO₄. The crude material was eluted with dichloromethane followed by ethyl acetate. The solvent was removed in vacuo and the residue was chromatographed on silica gel (EtOAc/Hex, 25:75, v/v to EtOAc/Hex, 50:50, v/v, TLC: EtOAc/Hex, 25:75, Rf=0.34) to afford B-Boc (325. mg, 21.0% yield) as a red oil: 1H NMR (CDCl₃) d 8.90-8.87 (m, 1H), 8.20-8.17 (m, 1H), 8.11-8.05 (m, 1H), 7.78-7.67 (m, 2H), 7.58-7.51 (m, 1H), 4.22 (br s, 2H), 1.47 (s, 9H).

### Compound B: 3-(quinolin-3-yl)prop-2-yn-1-amine

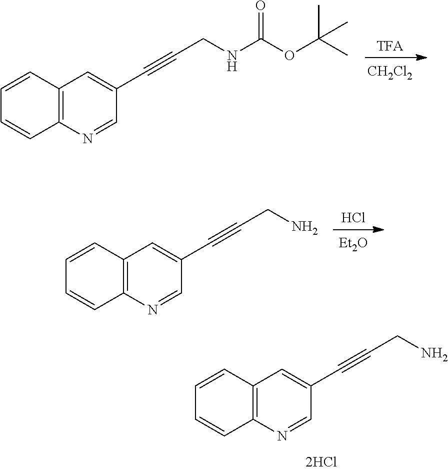

To a solution of B—BOC (112. mg, 0.390 mmol) in anhydrous dichloromethane (ca. 1.5 mL) was added trifluoroacetic acid (4 mL) and the resultant solution was stirred at ambient temperature for 1.5 hours. The mixture was diluted with 20 mL of water, transferred to a separatory funnel and the organics were discarded. The aqueous portion was washed with dichloromethane (2×40 mL), the pH was adjusted to ca. 10 using 10 N NaOH and extracted with dichloromethane (3×50 mL). The combined organics were dried over anhydrous MgSO₄, filtered and the solvent was removed in vacuo. This material was dissolved in 40 mL of ethyl ether and treated with 5 mL of ethereal HCl. The resulting precipitate was collected by centrifugation and dried in vacuo to afford B (58.6 mg, 68.1% yield) as a white solid: ¹H NMR (D₂O) d 8.77-8.68 (m, 1H), 7.63 (m, 1H), 8.60-8.47 (m, 1H), 7.80-7.50 (m, 4H), 4.07 (s, 2H); ¹³C NMR (75 MHz, CDCl₃) d 148.4, 147.9, 138.6, 136.3, 131.3, 129.8, 128.4, 116.8, 87.1, 81.6, 30.8.

### Compound C-Boc: tert-butyl (3-(4-methylpyridin-3-yl)prop-2-yn-1-yl)carbamate

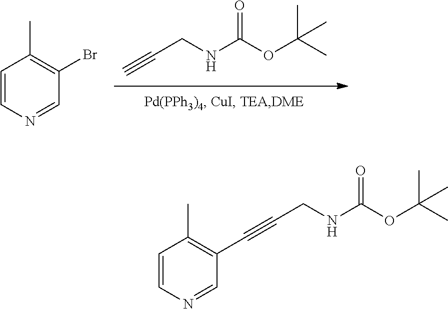

To a Biotage 2.0-5.0 mL microwave tube containing N-Boc-propargylamine (541. mg, 3.49 mmol) under a blanket of argon_{(g)} was added cuprous iodide (66. mg, 0.35 mmol) followed by tetrakis(triphenylphosphine)palladium(0) (200. mg, 0.174 mmol) followed by degassed 1-propanol (2.0 mL). To the vigorously stirred suspension was added sodium carbonate (481. mg, 4.53 mmol) dissolved in a minimum amount of water (ca. 1.5 mL). The tube was purged with argon_{(g)}, stirred at ambient temperature for ten minutes followed by the addition of a solution of 3-bromo-4-methylpyridine (600. mg, 3.49 mmol) in degassed 1-propanol. The tube was purged with argon_{(g)}, capped and placed in a Biotage Initiator+ microwave and heated to 100° C. for 15 minutes on normal absorption level. The contents of the flask were transferred to a sintered glass funnel containing anhydrous Na2SO4. The crude material was eluted with dichloromethane followed by methanol. The solvent was removed in vacuo and the residue was chromatographed on silica gel (EtOAc/Hex, 25:75, v/v to EtOAc/Hex, 50:50, v/v, TLC: EtOAc/Hex, 50:50, Rf=0.423) to C-Boc (455. mg, 53.0% yield) as a red oil: 1H NMR (500 MHz, CDCl₃) d 8.55 (s, 1H), 8.36 (s, 1H), 7.11 (s, 1H), 4.97 (br. s., 1H), 4.18 (br. s., 2H), 2.39 (s, 3H), 1.46 (s, 9H).

### Compound C: 3-(4-methylpyridin-3-yl)prop-2-yn-1-amine

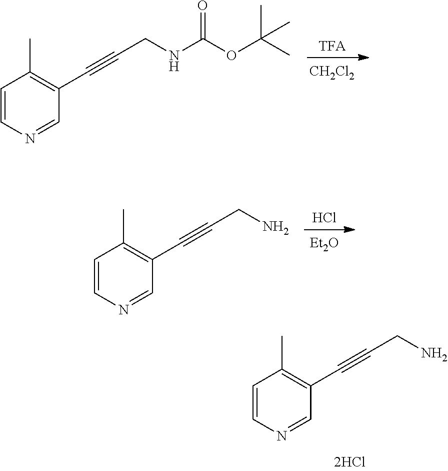

To a solution C-Boc (94. mg, 0.38 mmol) in anhydrous dichloromethane (ca. 1.5 mL) was added trifluoroacetic acid (3 mL) and the resultant solution was stirred at ambient temperature for 3 hours. To the mixture was added 3 mL of 1N HCl and the mixture was vigorously stirred for 10 min. The mixture was diluted with 20 mL of water, transferred to a separatory funnel and the organics were discarded. The aqueous portion was washed with dichloromethane (2×30 mL), the pH was adjusted to ca. 10 using 10 N NaOH and extracted with dichloromethane (3×35 mL). The combined organics were dried over anhydrous MgSO₄, filtered and the solvent was removed in vacuo. This material was dissolved in 40 mL of ethyl ether and treated with 5 mL of ethereal HCl. The resulting precipitate was collected by centrifugation and dried in vacuo to afford C (51. mg, 74% yield) as a white solid: 1H NMR (300 MHz, D₂O) d 8.66 (s, 1H), 8.45 (s, 1H), 7.77 (s, 1H), 4.07 (s, 2H), 2.56 (s, 3H); 13C NMR (75 MHz, CDCl₃) d 163.1, 144.8, 141.4, 128.8, 123.1, 91.8, 79.9, 30.8, 22.1.

### Compound D-Boc: tert-butyl (3-(5-methylpyridin-3-yl)prop-2-yn-1-yl)carbamate

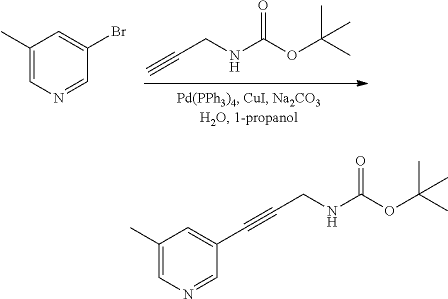

To a Biotage 2.0-5.0 mL microwave tube containing N-Boc-propargylamine (372. mg, 2.34 mmol) under a blanket of argon_{(g)} was added cuprous iodide (44.6 mg, 0.234 mmol) followed by tetrakis(triphenylphosphine)palladium(0) (135. mg, 0.117 mmol) followed by degassed 1-propanol (2.0 mL). To the vigorously stirred suspension was added sodium carbonate (410. mg, 3.87 mmol) dissolved in a minimum amount of water (ca. 1.3 mL). The tube was purged with argon_{(g)}, stirred at ambient temperature for ten minutes followed by the addition of a solution of 3-bromo-5-methylpyridine (402. mg, 2.34 mmol) in degassed 1-propanol. The tube was purged with argon_{(g)}, capped and placed in a Biotage Initiator+ microwave and heated to 100° C. for 15 minutes on normal absorption level. The contents of the flask were transferred to a sintered glass funnel containing anhydrous Na₂SO₄. The crude material was eluted with dichloromethane followed by methanol. The solvent was removed in vacuo and the residue was chromatographed on silica gel (EtOAc/Hex, 25:75, v/v, TLC: EtOAc/Hex, 25:75, Rf=0.116) to afford D-Boc (342. mg, 59.0% yield) as a yellow solid: 1H NMR (500 MHz, CDCl₃) d 8.45 (s, 1H), 8.35 (s, 1H), 7.49 (s, 1H), 5.05 (br. s., 1H), 4.14 (br. s., 2H), 2.28 (s, 3H), 1.45 (s, 9H).

### Compound D: 3-(5-methylpyridin-3-yl)prop-2-yn-1-amine

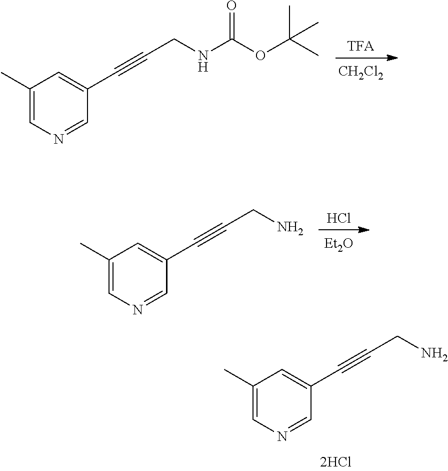

To a solution of D-Boc (76.4 mg, 0.310 mmol) in anhydrous dichloromethane (ca. 1.5 mL) was added trifluoroacetic acid (3 mL) and the resultant solution was stirred at ambient temperature for 3 hours. To the mixture was added 2 mL of 1N HCl and the mixture was vigorously stirred for 10 min. The mixture was diluted with 20 mL of water, transferred to a separatory funnel and the organics were discarded. The aqueous portion was washed with dichloromethane (2×30 mL), the pH was adjusted to ca. 10 using 10 N NaOH and extracted with dichloromethane (3×35 mL). The combined organics were dried over anhydrous MgSO₄, filtered and the solvent was removed in vacuo. The residue was chromatographed on silica gel (MeOH/CH₂Cl₂, 10/90, v/v, streak, Rf=0.114-0.409) to afford the compound as the free base. This material was dissolved in 40 mL of ethyl ether and treated with 5 mL of ethereal HCl. The resulting precipitate was collected by centrifugation and dried in vacuo to afford D (41.6 mg, 73.4% yield) as a white solid: 1H NMR (300 MHz, D₂O) d 8.57 (s, 1H), 8.45 (s, 1H), 8.31 (s, 1H), 3.97 (s, 2H), 2.35 (s, 3H); 13C NMR (75 MHz, D₂O) d 150.6, 142.3, 142.2, 140.3, 122.8, 86.3, 80.9, 30.8, 19.8.

### Compound E-Boc: tert-butyl (3-(6-methylpyridin-3-yl)prop-2-yn-1-yl)carbamate

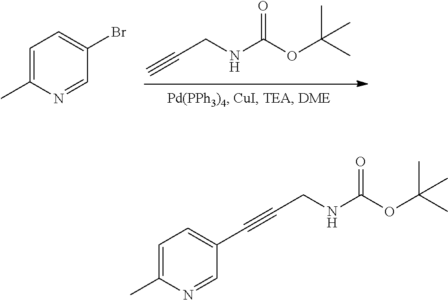

To a Biotage 2.0-5.0 mL microwave tube containing N-Boc-propargylamine (474 mg, 2.98 mmol) under a blanket of argon_{(g)} was added cuprous iodide (56.6 mg, 0.297 mmol) followed by tetrakis(triphenylphosphine)palladium(0) (172 mg, 0.149 mmol) followed by degassed 1-propanol (2.0 mL). To the vigorously stirred suspension was added sodium carbonate (410 mg, 3.87 mmol) dissolved in a minimum amount of water (ca. 1.3 mL). The tube was purged with argon_{(g)}, stirred at ambient temperature for ten minutes followed by the addition of a solution of 3-bromo-6-methylpyridine (512 mg, 2.97 mmol) in degassed 1-propanol. The tube was purged with argon_{(g)}, capped and placed in a Biotage Initiator+ microwave and heated to 100° C. for 15 minutes on normal absorption level. The contents of the flask were transferred to a sintered glass funnel containing anhydrous Na₂SO₄. The crude material was eluted with dichloromethane followed by methanol. The solvent was removed in vacuo and the residue was chromatographed on silica gel (EtOAc/Hex, 25:75, v/v to EtOAc/Hex, 50:50, v/v, TLC: EtOAc/Hex, 50:50, Rf=0.478) to afford E-Boc (349 mg, 47.6% yield) as a dark red oil: 1H NMR (500 MHz, CDCl₃) d=8.52 (d, I=1.58 Hz, 1H), 7.55 (dd, J=2.21, 7.88 Hz, 1H), 7.07 (d, J=8.20 Hz, 1H), 2.52 (s, 2H), 1.50 (s, 9H).

### Compound E: 3-(6-methylpyridin-3-yl)prop-2-yn-1-amine

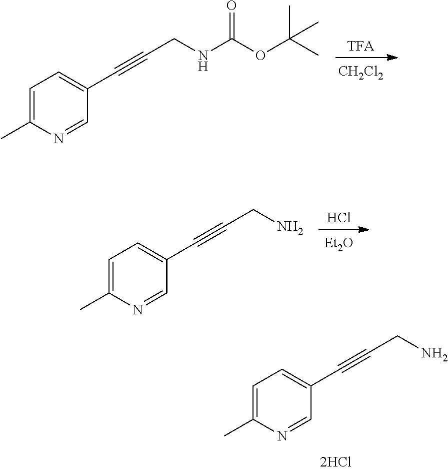

To a solution of E-Boc (282 mg, 1.14 mmol) in anhydrous dichloromethane (ca. 10 mL) was added trifluoroacetic acid (1 mL) and the resultant solution was stirred at ambient temperature for 15 minutes. To the mixture was added 50 mL of 1N HCl and the mixture was vigorously stirred for 10 minutes. The mixture was transferred to a separatory funnel, the organics were discarded and the aqueous phase was washed with dichloromethane (2×25 mL). The aqueous portion was transferred to an Erlenmeyer flask, the pH was adjusted to ca. 10 using 10 N NaOH, ca. 50 mL of brine was added followed by ca. 75 mL of EtOAc and the mixture was vigorously stirred for 5-10 minutes. The organics were collected and the aqueous portion was extracted with EtOAc (100 mL). The combined organics were dried over anhydrous MgSO₄, filtered and the solvent was removed in vacuo. The residue was chromatographed on silica gel (MeOH/CH₂Cl₂, 10/90, v/v, Rf=0.225) to afford E (107. mg, 64.1% yield) as a red oil: 1H NMR (300 MHz, D₂O) d=8.59 (d, I=1.9 Hz, 1H), 8.29 (dd, J=2.0, 8.4 Hz, 1H), 7.69 (dd, J=0.6, 8.5 Hz, 1H), 3.96 (s, 2H), 2.60 (s, 3H); 13C NMR (CDCl₃) d 156.74, 150.77, 145.62, 130.42, 121.69, 89.08, 82.03, 32.02, 21.66; HRMS (ESI) m/z calculated for C9H11N2 [M+H]+ 147.0922, found 147.0947.

### Compound F-Boc: tert-butyl (3-(2-methylpyridin-3-yl)prop-2-yn-1-yl)carbamate

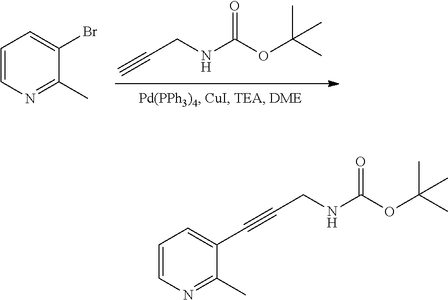

To a Biotage 2.0-5.0 mL microwave tube containing N-Boc-propargylamine (413 mg, 2.66 mmol) under a blanket of argon_{(g)} was added degassed DME/EtOH 50:50 (2.5 mL) followed by cuprous iodide (46.0 mg, 0.242 mmol) followed by tetrakis(triphenylphosphine)palladium(0) (290 mg, 0.25 mmol). To the vigorously stirred suspension was added triethylamine (734 mg, 7.26 mmol). The tube was purged with argon_{(g)}, stirred at ambient temperature for ten minutes followed by the addition of a solution of 3-bromo-2-methylpyridine (416 mg, 2.42 mmol). The tube was purged with argon_{(g)}, capped and placed in a Biotage Initiator+ microwave and heated to 150° C. for 15 minutes on normal absorption level. The contents of the flask were filtered through a compressed bed of celite and the crude material was eluted with dichloromethane. The solvent was removed in vacuo and the residue was chromatographed on silica gel (EtOAc/Hex, 25:75, v/v Rf=0.167) to afford F-Boc (192 mg, 32.0% yield) as a red oil: 1H NMR (500 MHz, CDCl₃) d=8.34 (d, J=3.6 Hz, 1H), 7.55 (dd, J=1.5, 7.7 Hz, 1H), 6.98 (dd, J=4.9, 7.5 Hz, 1H), 5.31 (br. s., 1H), 4.13 (d, J=4.9 Hz, 2H), 2.56 (s, 3H), 1.40 (s, 9H).

### Compound F: 3-(2-methylpyridin-3-yl)prop-2-yn-1-amine

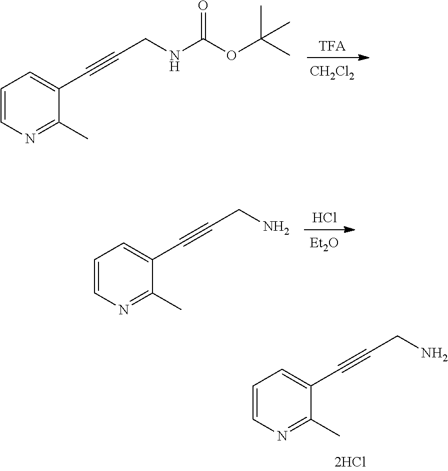

To a solution of F-Boc (192. mg, 0.780 mmol) in anhydrous dichloromethane (ca. 1.5 mL) was added trifluoroacetic acid (3 mL) and the resultant solution was stirred at ambient temperature for 2 hours. To the mixture was added 4 mL of 1N HCl and the mixture was vigorously stirred for 10 minutes. The mixture was diluted with 20 mL of water, transferred to a separatory funnel and the organics were discarded. The aqueous portion was washed with dichloromethane (2×40 mL). The aqueous portion was washed with dichloromethane (2×30 mL), the pH was adjusted to ca. 10 using 10 N NaOH and extracted with dichloromethane (3×35 mL). The combined organics were dried over anhydrous MgSO₄, filtered and the solvent was removed in vacuo. The residue was chromatographed on silica gel (MeOH/CH₂Cl₂, 10/90, v/v, Rf=0.227) This material was dissolved in 40 mL of ethyl ether and treated with 5 mL of ethereal HCl. The resulting precipitate was collected by centrifugation and dried in vacuo to afford F (109 mg, 77.0% yield) as a white solid: 1H NMR (300 MHz, D₂O) d=8.43-8.35 (m, 2H), 7.71-7.66 (m, 1H), 4.02 (s, 2H), 2.68 (s, 3H); 13C NMR (75 MHz, CDCl₃) d=157.5, 149.8, 141.2, 125.5, 123.4, 91.6, 80.4, 30.8, 19.8.

### Compound G-Boc: tert-butyl (3-(pyrimidin-5-yl)prop-2-yn-1-yl)carbamate

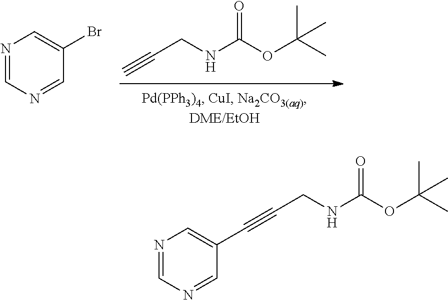 [SMILES: Brc1cncnc1 ; C#CCNC(=O)OC(C)(C)C ; CC(C)(C)OC(=O)NCC#Cc1cncnc1]

To a Biotage 2.0-5.0 mL microwave tube containing N-Boc-propargylamine (633 mg, 4.08 mmol) under a blanket of argon_{(g)} was added cuprous iodide (77.0 mg, 0.408 mmol) followed by tetrakis(triphenylphosphine)palladium(0) (260. mg, 0.225 mmol) followed by degassed 1-propanol (1.5 mL). To the vigorously stirred suspension was added sodium carbonate (562 mg, 5.3 mmol) dissolved in degassed water (ca. 2.0 mL). The tube was purged with argon_{(g)}, stirred at ambient temperature for ten minutes followed by the addition of a solution of 5-bromo-pyrimidine (645 mg, 4.08 mmol) in hot degassed 1-propanol (1.5 mL). The tube was purged with argon_{(g)}, capped and placed in a Biotage Initiator+ microwave and heated to 100° C. for 15 minutes on normal absorption level. The contents of the flask were transferred to a sintered glass funnel containing anhydrous Na₂SO₄. The crude material was eluted with dichloromethane followed by methanol. The solvent was removed in vacuo and the residue was chromatographed on silica gel (EtOAc/Hex, 25:75, v/v Rf=0.33) to afford G-Boc (560 mg, 59.0% yield) as a red oil: 1H NMR (500 MHz, CDCl₃) d 9.06 (s, 1H), 8.69 (s, 2H), 7.21 (m, 2H), 5.29 (br s, 1H), 4.13 (s, 2H) 1.40 (s, 9H).

### Compound G: 3-(5-methylpyridin-3-yl)prop-2-yn-1-amine

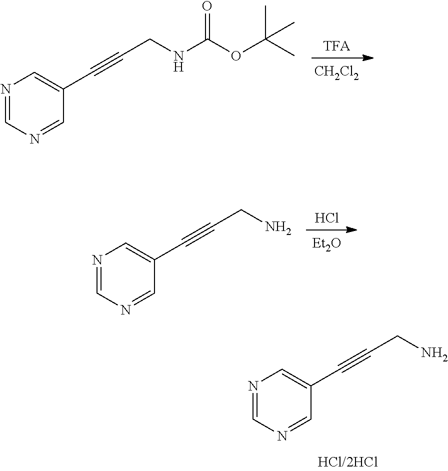

To a solution of G-Boc (118 mg, 0.51 mmol) in anhydrous dichloromethane (ca. 1.5 mL) was added trifluoroacetic acid (3 mL) and the resultant solution was stirred at ambient temperature for 2 hours. To the mixture was added 3 mL of 1N HCl and the mixture was vigorously stirred for 10 minutes. The mixture was diluted with 20 mL of water, transferred to a separatory funnel and the organics were discarded. The aqueous portion was washed with dichloromethane (2×30 mL), the pH was adjusted to ca. 10 using 10 N NaOH and extracted with dichloromethane (3×35 mL). The combined organics were dried over anhydrous MgSO₄, filtered and the solvent was removed in vacuo. The residue was passed through a short pad of silica gel and this material was dissolved in 40 mL of ethyl ether and treated with 5 mL of ethereal HCl. The resulting precipitate was collected by centrifugation and dried in vacuo to afford G (109. mg, 77% yield) as a white solid: as a 2:1 mixture of the monohydrochloride to dihydrochloride: 1H NMR (300 MHz, D₂O) d=8.94 (mono-HCl, s, 1H), 8.75 (mono-HCl, s, 2H), 8.15 (di-HCl, s, 1H), 6.87 (di-HCl, s, 1H), 7.21 (m, 2H), 5.59 (di-HCl, s, 1H), 3.94 (mono-HCl, s, 2H), 3.82 (di-HCl, s, 2H).

Free Base NMR: 1H NMR (300 MHz, CDCl₃) d 10.04 (s, 1H), 9.82-9.56 (m, 2H), 2.47 (br. s., 2H).

### Compound H-Boc: tert-butyl (3-(4-ethylpyridin-3-yl)prop-2-yn-1-yl)carbamate

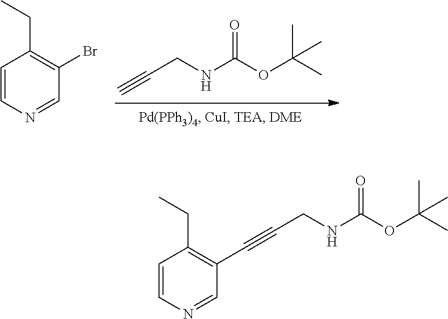

To a Biotage 2.0-5.0 mL microwave tube containing N-Boc-propargylamine (224 mg, 1.44 mmol) under a blanket of argon_{(g)} was added degassed DME/EtOH 50:50 (0.5 mL) followed by a slurry of tetrakis(triphenylphosphine)palladium(0) (280. mg, 0.242 mmol) in degassed DME/EtOH 50:50 (1.5 mL), followed by cuprous iodide (27.0 mg, 0.144 mmol). To the vigorously stirred suspension was added sodium carbonate (300. mg, 1.57 mmol) dissolved in a minimum amount of degassed water (ca. 1.5 mL), the tube was purged with argon_{(g)}, stirred at ambient temperature for ten minutes, followed by the addition of a solution of 3-bromo-4-ethylpyridine (269. mg, 1.44 mmol). The tube was purged with argon_{(g)}, capped and placed in a Biotage Initiator+ microwave and heated to 150° C. for 15 minutes on normal absorption level. The contents of the flask were transferred to a sintered glass funnel containing anhydrous Na₂SO₄ and the crude material was eluted with dichloromethane. The solvent was removed in vacuo and the residue was chromatographed on silica gel (EtOAc/Hex, 25:75, v/v Rf=0.2) to afford H-Boc (141. mg, 50.6% yield) as a yellow oil: 1H NMR (500 MHz, CDCl₃) d=8.54 (s, 1H), 8.39 (d, J=5.4 Hz, 1H), 7.10 (d, J=5.0 Hz, 1H), 5.10 (br, s, 1H), 4.17 (s, 2H), 2.74 (q, J=7.6 Hz, 2H), 1.45 (s, 9H), 1.21 (t, J=7.6 Hz, 3H).

### Compound H: 3-(4-ethylpyridin-3-yl)prop-2-yn-1-amine

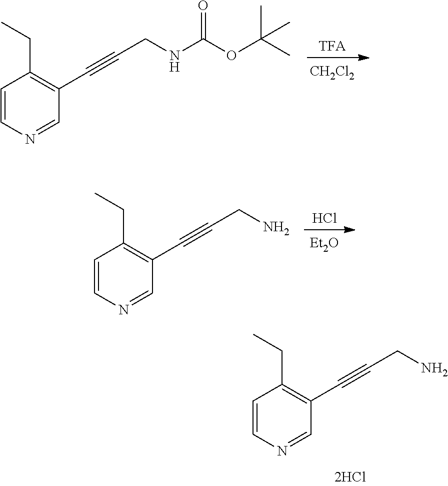

To solution I-Boc (69.5 mg, 0.260 mmol) in anhydrous dichloromethane (ca. 3.0 mL) was added trifluoroacetic acid (3 mL) and the resultant solution was stirred at ambient temperature for 2 hours. To the mixture was added 3 mL of 1N HCl and the mixture was vigorously stirred for 10 minutes. The mixture was diluted with 20 mL of water, and transferred to a separatory funnel, the organics were discarded and the aqueous phase was washed with dichloromethane (2×40 mL). The mixture was diluted with 20 mL of water, transferred to a separatory funnel and the organics were discarded. The aqueous portion was washed with dichloromethane (2×40 mL), the pH was adjusted to ca. 10 using 10 N NaOH and extracted with dichloromethane (2×40 mL). The combined organics were dried over anhydrous MgSO₄, filtered and the solvent was removed in vacuo. The combined organics were dried over anhydrous MgSO₄, filtered and the solvent was removed in vacuo. This material was dissolved in 40 mL of ethyl ether and treated with 5 mL of ethereal HCl. The resulting precipitate was collected by centrifugation and dried in vacuo to afford H (49.0 mg, 93% yield) as an off white solid: 1H NMR (500 MHz, D₂O) d=8.69 (s, 1H), 8.51 (d, J=5.7 Hz, 1H), 7.84 (d, J=6.0 Hz, 1H), 4.07 (s, 2H), 2.93 (q, J=7.6 Hz, 2H), 1.18 (t, J=7.6 Hz, 3H); 13C NMR (CDCl₃) d 168.7, 144.8, 141.4, 127.4, 122.8, 91.8, 79.5, 30.8, 29.0, 13.2.

### Compound I-Boc: tert-butyl (3-(4-propylpyridin-3-yl)prop-2-yn-1-yl)carbamate

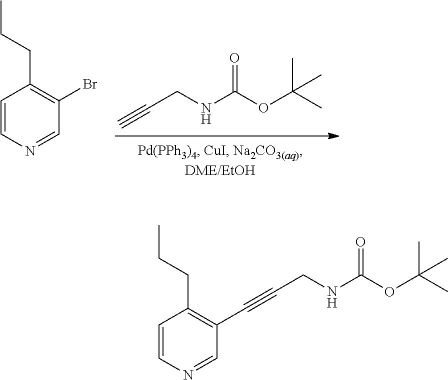

To a Biotage 2.0-5.0 mL microwave tube containing N-Boc-propargylamine (147 mg, 0.950 mmol) under a blanket of argon_{(g)} was added degassed DME/EtOH 50:50 (3.0 mL) followed by a slurry of tetrakis(triphenylphosphine)palladium(0) (145 mg, 0.125 mmol) in degassed DME/EtOH 50:50 (1.0 mL) followed by cuprous iodide (30.0 mg, 0.157 mmol). To the vigorously stirred suspension was added sodium carbonate (215 mg, 2.02 mmol) dissolved in degassed water (ca. 1.5 mL). The tube was purged with argon_{(g)}, stirred at ambient temperature for ten minutes followed by the addition of a solution of 3-bromo-4-propylpyridine (190. mg, 0.950 mmol). The tube was purged with argon_{(g)}, capped and placed in a Biotage Initiator+ microwave and heated to 150° C. for 15 minutes on normal absorption level. The contents of the flask were transferred to a sintered glass funnel containing anhydrous Na₂SO₄ and the crude material was eluted with dichloromethane. The solvent was removed in vacuo and the residue was chromatographed on silica gel (EtOAc/Hex, 25:75, v/v Rf=0.1) to afford I-Boc (69.0 mg, 29% yield) as a red oil: 1H NMR (500 MHz, CDCl₃) d=8.55 (s, 1H), 8.38 (d, J=5.0 Hz, 1H), 7.08 (d, J=4.7 Hz, 1H), 4.94 (br. s., 1H), 4.27-4.07 (m, 2H), 2.79-2.59 (m, 2H), 1.75-1.56 (m, 2H), 1.52-1.39 (m, 9H), 1.03-0.84 (m, 3H).

### Compound I: 3-(4-propylpyridin-3-yl)prop-2-yn-1-amine

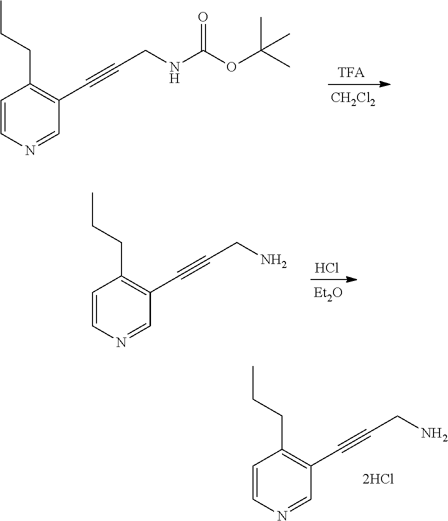

To a solution of I-Boc (69. mg, 0.25 mmol) in anhydrous dichloromethane (ca. 1.5 mL) was added trifluoroacetic acid (3 mL) and the resultant solution was stirred at ambient temperature for 3 hours. To the mixture was added 3 mL of 1N HCl and the mixture was vigorously stirred for 10 minutes. The mixture was diluted with 20 mL of water, transferred to a separatory funnel and the organics were discarded. The aqueous portion was washed with dichloromethane (2×30 mL), the pH was adjusted to ca. 10 using 10 N NaOH and extracted with dichloromethane (2×40 mL). The combined organics were dried over anhydrous MgSO₄, filtered and the solvent was removed in vacuo. This material was dissolved in 40 mL of ethyl ether and treated with 5 mL of ethereal HCl. The resulting precipitate was collected by centrifugation and dried in vacuo to afford I (37. mg, 70% yield) as an off white solid: 1H NMR (500 MHz, D₂O) d 8.68 (br. s., 1H), 8.48 (d, J=5.0 Hz, 1H), 7.81 (d, J=6.0 Hz, 1H), 4.09-4.01 (m, 2H), 2.89 (t, J=7.6 Hz, 2H), 1.73-1.51 (m, 2H), 0.92-0.70 (m, 3H).

### Compound J-Boc: tert-butyl (3-(4-phenylpyridin-3-yl)prop-2-yn-1-yl)carbamate

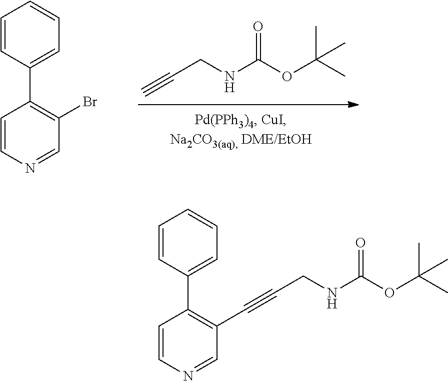 [SMILES: Brc1cnccc1-c1ccccc1 ; C#CCNC(=O)OC(C)(C)C ; CC(C)(C)OC(=O)NCC#Cc1cnccc1-c1ccccc1]

To a Biotage 2.0-5.0 mL microwave tube containing N-Boc-propargylamine (167 mg, 1.07 mmol) under a blanket of argon_{(g)} was added degassed DME/EtOH 50:50 (1.0 mL) followed by a slurry of tetrakis(triphenylphosphine)palladium(0) (102 mg, 0.080 mmol) in degassed DME/EtOH 50:50 (1.0 mL) followed by cuprous iodide (25.0 mg, 0.131 mmol). To the vigorously stirred suspension was added sodium carbonate (253 mg, 2.38 mmol) dissolved in degassed water (ca. 1.5 mL). The tube was purged with argon_{(g)}, stirred at ambient temperature for ten minutes followed by the addition of a solution of 3-bromo-4-phenylpyridine (252 mg, 1.07 mmol). The tube was purged with argon_{(g)}, capped and placed in a Biotage Initiator+ microwave and heated to 120° C. for 15 minutes on normal absorption level. The contents of the flask were transferred to a sintered glass funnel containing anhydrous Na₂SO₄. The crude material was eluted with dichloromethane. The solvent was removed in vacuo and the residue was chromatographed on silica gel (EtOAc/Hex, 25:75, v/v Rf=0.16) to afford J-Boc (219.9 mg, 66% yield) as a red oil: 1H NMR (500 MHz, CDCl₃) d=8.73 (br. s., 1H), 8.55 (br. s., 1H), 7.68-7.57 (m, 2H), 7.53-7.38 (m, 3H), 7.36-7.27 (m, 1H), 4.73 (br. s., 1H), 4.11-3.99 (m, 2H), 1.48-1.42 (m, 9H).

### Compound J: 3-(4-phenylpyridin-3-yl)prop-2-yn-1-amine

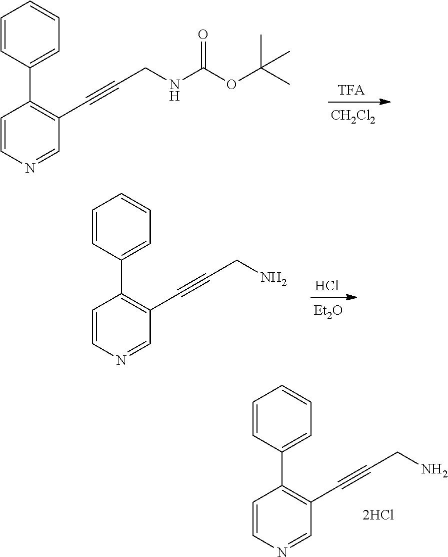

To a solution of J-Boc (168 mg, 0.540 mmol) in anhydrous dichloromethane (ca. 1.5 mL) was added trifluoroacetic acid (3 mL) and the resultant solution was stirred at ambient temperature for 3 hours. To the mixture was added 2 mL of 1N HCl and the mixture was vigorously stirred for 10 minutes. The mixture was diluted with 20 mL of water, transferred to a separatory funnel and the organics were discarded. The aqueous portion was washed with dichloromethane (2×30 mL), the pH was adjusted to ca. 10 using 10 N NaOH and extracted with dichloromethane (2×35 mL). The combined organics were dried over anhydrous MgSO₄, filtered and the solvent was removed in vacuo. This material was dissolved in 40 mL of ethyl ether and treated with 5 mL of ethereal HCl. The resulting precipitate was collected by centrifugation and dried in vacuo to afford J (95.7 mg, 71% yield) as a white solid: 1H NMR (300 MHz, CDCl₃) d 8.80 (s, 1H), 8.66-8.45 (m, 1H), 7.81 (d, J=6.2 Hz, 1H), 7.71-7.52 (m, 2H), 7.52-7.27 (m, 3H), 3.87 (s, 2H); 13C NMR (CDCl₃) d 161.3, 146.5, 141.4, 135.8, 132.7, 130.3, 130.2, 128.5, 121.2, 90.7, 80.8, 30.8.

### Compound K-Boc: tert-butyl (3-(4-methoxypyridin-3-yl)prop-2-yn-1-yl)carbamate

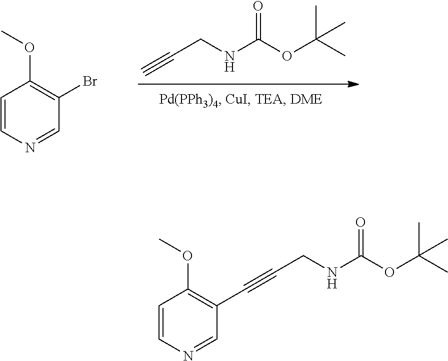

To a Biotage 2.0-5.0 mL microwave tube containing N-Boc-propargylamine (212 mg, 1.37 mmol) under a blanket of argon_{(g)} was added degassed DME/EtOH 50:50 (0.3 mL) followed by a slurry of tetrakis(triphenylphosphine)palladium(0) (109 mg, 0.094 mmol) in degassed DME/EtOH 50:50 (2.0 mL), followed by cuprous iodide (29.0 mg, 0.152 mmol). To the vigorously stirred suspension was added sodium carbonate (290 mg, 2.73 mmol) dissolved in a minimum amount of degassed water (ca. 1.5 mL), the tube was purged with argon_{(g)}, stirred at ambient temperature for ten minutes, followed by the addition of a solution of 3-bromo-4-methoxypyridine (257. mg, 1.37 mmol). The tube was purged with argon_{(g)}, capped and placed in a Biotage Initiator+ microwave and heated to 150° C. for 15 minutes on normal absorption level. The contents of the flask were transferred to a sintered glass funnel containing anhydrous Na₂SO₄ and the crude material was eluted with dichloromethane. The solvent was removed in vacuo and the residue was chromatographed on silica gel (EtOAc/Hex, 25:75, v/v Rf=0.23) to afford K-Boc (272. mg, 76% yield) as a yellow oil: 1H NMR (500 MHz, CDCl₃) d=8.48 (s, 1H), 8.40 (d, J=5.7 Hz, 1H), 6.77 (d, J=6.0 Hz, 1H), 4.93 (br s, 1H), 4.20 (br s, 2H), 3.93-3.88 (m, 3H), 1.46 (s, 9H).

### Compound K: 3-(4-methoxypyridin-3-yl)prop-2-yn-1-amine

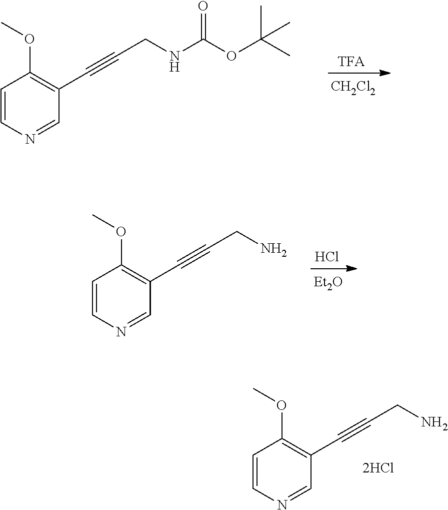

To a solution of K-Boc (272 mg, 1.03 mmol) in anhydrous dichloromethane (ca. 3.0 mL) was added trifluoroacetic acid (4 mL) and the resultant solution was stirred at ambient temperature for 3 hours. To the mixture was added 3 mL of 1N HCl and the mixture was vigorously stirred for 10 minutes. The mixture was diluted with 20 mL of water, transferred to a separatory funnel and the organics were discarded. The aqueous portion was washed with dichloromethane (2×30 mL), the pH was adjusted to ca. 10 using 10 N NaOH and extracted with dichloromethane (2×40 mL). The combined organics were dried over anhydrous MgSO₄, filtered and the solvent was removed in vacuo. The combined organics were dried over anhydrous MgSO₄, filtered and the solvent was removed in vacuo. This material was dissolved in 40 mL of ethyl ether and treated with 5 mL of ethereal HCl. The resulting precipitate was collected by centrifugation and dried in vacuo to afford the product K (140. mg, 68% yield) as a white solid: 1H NMR (500 MHz, CDCl₃) d=8.44 (s, 1H), 8.35 (d, J=5.7 Hz, 1H), 6.74 (d, J=6.0 Hz, 1H), 3.87 (s, 3H), 3.65 (s, 2H); 13C NMR (CDCl₃) d 161.3, 146.5, 141.4, 135.8, 132.7, 130.3, 130.2, 128.5, 121.2, 90.7, 80.8, 30.8.

### Compound L-Boc: tert-Butyl (3-(4-chloropyridin-3-yl)prop-2-yn-1-yl)carbamate

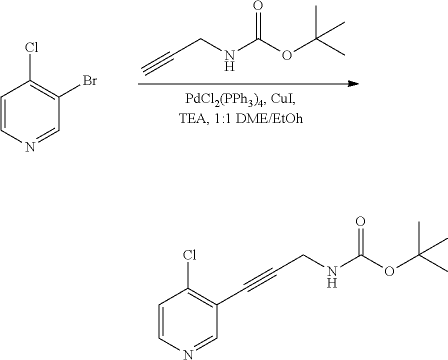 [SMILES: Clc1ccncc1Br ; C#CCNC(=O)OC(C)(C)C ; CC(C)(C)OC(=O)NCC#Cc1cnccc1Cl]

To a 5 mL microwave vial (Biotage) was added 3-bromo-4-chloropyridine (267 mg, 1.39 mmol), tert-butyl prop-2-ynyl-carbamate (232 mg, 1.49 mmol), CuI (23.0 mg, 0.121 mmol) and bis(triphenylphosphine)palladium(II) dichloride (43.0 mg, 0.0613 mmol). The vial was purged with argon for 5 minutes followed by addition of degassed DME/EtOH 50:50 (2.0 mL) and degassed Et3N (600. mL, 4.30 mmol). The vial was capped and placed in a Biotage Initiator+ microwave and heated to 140° C. for 15 minutes on normal absorption. The contents of the flask were cooled to rt, transferred to a separatory funnel, diluted with EtOAc (30 mL), washed with water (15 mL), followed by saturated NaCl (15 mL), dried over Na₂SO₄, gravity filtered, the solvent was removed in vacuo and the residue was chromatographed on silica gel (EtOAc/Hex, 0:100, v/v to EtOAc/Hex, 100:0, v/v, TLC: 50% EtOAc/hexane, Rf=0.52) to afford L-Boc (113. mg, 30% yield) as a brown syrup: 1H NMR (500 MHz, CDCl₃) d 8.67 (bs, 1H), 8.45 (bs, 1H), 7.35 (s, 1H), 5.30 (bs, 1H), 4.24 (m, 2H), 1.48 (s, 9H).

### Compound L: 3-(4-chloropyridin-3-yl)prop-2-yn-1-amine Dihydrochloride

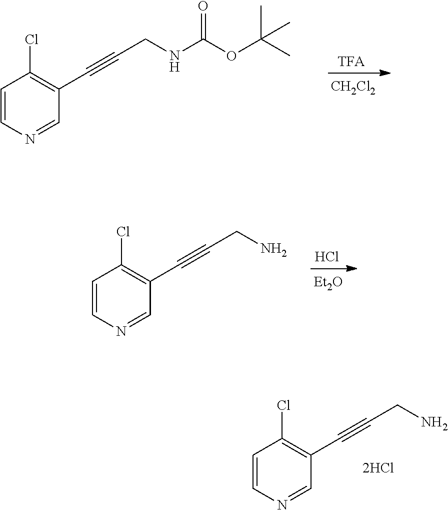

To a solution of L-Boc (113 mg, 0.424 mmol) in dichloromethane (0.75 mL), cooled to 0° C. in an external ice-water bath, was added trifluoroacetic acid (0.75 mL). The ice bath was removed and the resultant solution was stirred at ambient temperature for 2 hours. The reaction mixture was subsequently chilled in an external ice-water bath followed by dropwise adding saturated sodium carbonate (7.5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford L (58.0 mg, 57% yield) as a light yellow solid. 1H NMR (500 MHz, D₂O) d 8.93 (s, 1H), 8.67 (d, J=6.3 Hz, 1H), 8.09 (d, J=6.3 Hz, 1H), 4.20 (s, 2H); 13C NMR (125 MHz, D₂O) d 155.4, 147.9, 144.0, 128.7, 123.1, 92.9, 78.8, 30.8. HRMS calculated for C9H9ClN [M+H]+, 167.0376, found 167.0378, UPLC (254 nm)>95%.

### Compound M-Boc: tert-Butyl [3-(4-furan-2-yl-pyridin-3-yl)-prop-2-ynyl]-carbamate

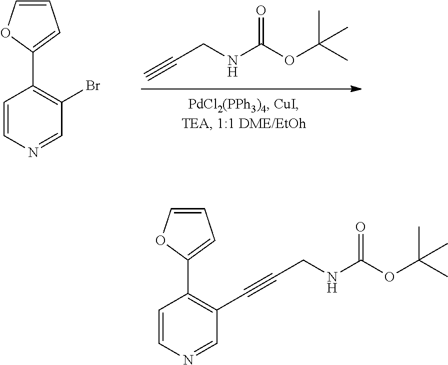 [SMILES: Brc1cnccc1-c1ccco1 ; C#CCNC(=O)OC(C)(C)C ; CC(C)(C)OC(=O)NCC#Cc1cnccc1-c1ccco1]

To a 5 mL microwave vial (Biotage) was added 3-bromo-4-(furan-2-yl)pyridine (87.0 mg, 0.388 mmol), tert-butyl prop-2-ynyl-carbamate (61.0 mg, 0.393 mmol), CuI (20.0 mg, 0.105 mmol) and bis(triphenylphosphine)palladium(II) dichloride (27.0 mg, 0.0385 mmol). The vial was purged with argon for 5 minutes followed by addition of degassed DME/EtOH 50:50 (2.0 mL) and degassed Et3N (165 mL, 1.18 mmol). The vial was capped and placed in a Biotage Initiator+ microwave and heated to 140° C. for 15 minutes on normal absorption. The contents of the flask were cooled to rt, transferred to a separatory funnel, diluted with CH₂Cl₂ (25 mL), washed with water (20 mL), followed by saturated NaCl (20 mL), dried over Na₂SO₄, gravity filtered, the solvent was removed in vacuo and the residue was chromatographed on silica gel (EtOAc/Hex, 10:90, v/v to EtOAc/Hex, 50:50, v/v, TLC: 50% EtOAc/hexane, Rf=0.56) to afford M-Boc (39.0 mg, 34% yield) as a semisolid: 1H NMR (500 MHz, CDCl₃) d 8.20-9.20 (m, 2H), 7.66 (s, 1H), 7.54 (s, 1H), 7.50 (m, 1H), 6.49 (dd, J=3.6 Hz, J=1.8 Hz, 1H), 5.07 (bs, 1H), 4.19 (d, J=5.3 Hz, 2H), 1.41 (s, 9H).

### Compound M: 3-(4-Furan-2-yl-pyridin-3-yl)-prop-2-ynylamine Dihydrochloride

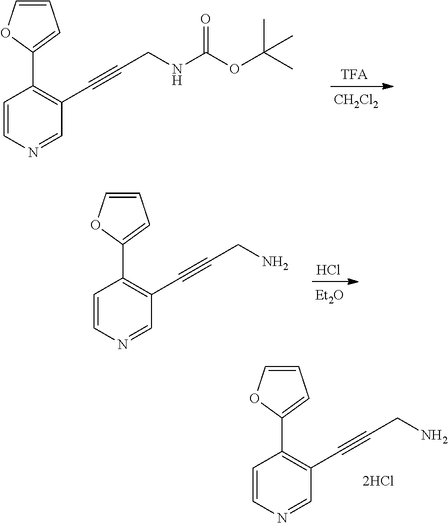

To a solution of M-BOC (39.0 mg, 0.131 mmol) in dichloromethane (0.75 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford M (32.0 mg, 90% yield) as a yellow solid: 1H NMR (500 MHz, D₂O) d 8.85 (s, 1H), 8.60 (dd, J=6.6 Hz, J=0.6 Hz, 1H), 8.29 (d, J=6.6 Hz, 1H), 8.01 (d, J=3.8 Hz, 1H), 7.94 (d, J=1.6 Hz, 1H), 6.82 (dd, J=3.8 Hz, J=1.6 Hz, 1H), 4.27 (s, 2H); 13C NMR (125 MHz, D₂O) d 149.7, 148.2, 147.2, 145.9, 140.9, 122.0, 121.2, 115.0 (2 C), 91.9, 81.3, 30.8. HRMS calculated for C12H11N2O [M+H]+, 199.0871, found 199.0889, UPLC (254 nm)=94%.

### Compound N-Boc: tert-Butyl [3-(4-furan-3-yipyridin-3-yl)-prop-2-ynyl]-carbamate

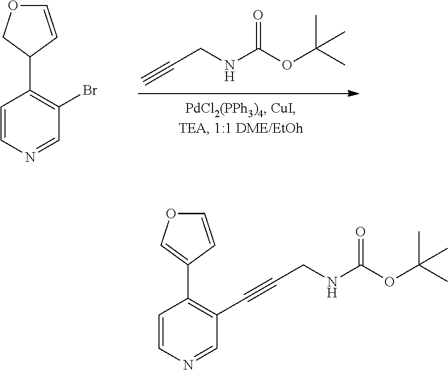 [SMILES: Brc1cnccc1C1C=COC1 ; C#CCNC(=O)OC(C)(C)C ; CC(C)(C)OC(=O)NCC#Cc1cnccc1-c1ccoc1]

To a 5 mL microwave vial (Biotage) was added 3-bromo-4-(furan-3-yl)pyridine (83.0 mg, 0.370 mmol), tert-butyl prop-2-ynyl-carbamate (59.0 mg, 0.380 mmol), CuI (9.0 mg, 0.0472 mmol) and bis(triphenylphosphine)palladium(II) dichloride (25.0 mg, 0.0356 mmol). The vial was purged with argon for 5 minutes followed by addition of degassed DME/EtOH 50:50 (2.0 mL) and degassed Et3N (155. mL, 1.11 mmol). The vial was capped and placed in a Biotage Initiator+ microwave and heated to 140° C. for 15 minutes on normal absorption. The contents of the flask were cooled to rt, transferred to a separatory funnel, diluted with EtOAc (25 mL), washed with water (25 mL), followed by saturated NaCl (20 mL), dried over Na₂SO₄, gravity filtered, the solvent was removed in vacuo and the residue was chromatographed on silica gel (EtOAc/Hex, 10:90, v/v to EtOAc/Hex, 50:50, v/v, TLC: 50% EtOAc/hexane, Rf=0.44) to afford N-Boc. (40.0 mg, 36% yield) as a yellow solid: 1H NMR (500 MHz, CDCl₃) d 8.66 (s, 1H), 8.48 (d, J=5.3 Hz, 1H), 8.30 (s, 1H), 7.52 (dd, J=1.6 Hz, 1H), 7.32 (d, J=5.3 Hz, 1H), 6.87 (m, 1H), 4.97 (bs, 1H), 4.21 (d, J=5.2 Hz, 2H), 1.48 (s, 9H).

### Compound N: 3-(4-Furan-3-yl-pyridin-3-yl)-prop-2-ynylamine Dihydrochloride

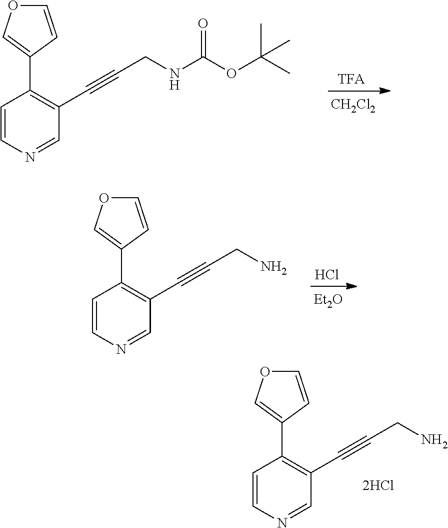

To a solution of N—BOC (40.0 mg, 0.134 mmol) in dichloromethane (0.75 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford N (14.0 mg, 39% yield) as a yellow solid: 1H NMR (500 MHz, D₂O) d 8.88 (s, 1H), 8.67 (s, 1H), 8.63 (d, J=6.4 Hz, 1H), 8.11 (d, J=6.4 Hz, 1H), 7.74 (m, 1H), 7.14 (m, 1H), 4.22 (s, 2H); 13C NMR (125 MHz, D₂O) d 151.8, 147.7, 147.0, 146.2, 141.2, 125.7, 122.3, 118.6, 110.2, 91.6, 81.5, 30.8. HRMS calculated for C12H11N2O [M+H]+, 199.0871, found 199.0890, UPLC (254 nm)=90%.

### Compound O-Boc: tert-Butyl (3-([3,4′-bipyridin]-3′-yl)prop-2-yn-1-yl)carbamate

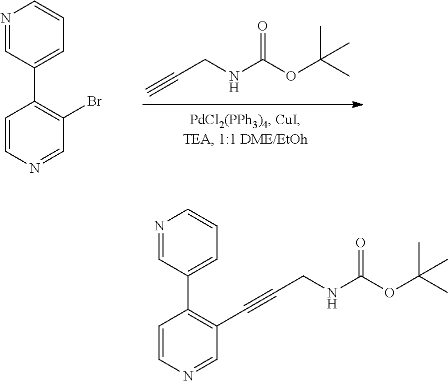 [SMILES: Brc1cnccc1-c1cccnc1 ; C#CCNC(=O)OC(C)(C)C ; CC(C)(C)OC(=O)NCC#Cc1cnccc1-c1cccnc1]

To a 5 mL microwave vial (Biotage) was added 3′-bromo-3, 4′-bipyridine (116. mg, 0.493 mmol), tert-butyl prop-2-ynyl-carbamate (76.5 mg, 0.493 mmol), CuI (12.0 mg, 0.0630 mmol) and bis(triphenylphosphine)palladium(II) dichloride (36.0 mg, 0.0513 mmol). The vial was purged with argon for 5 minutes followed by addition of degassed DME/EtOH 50:50 (2.0 mL) and degassed Et3N (205. mL, 1.47 mmol). The vial was capped and placed in a Biotage Initiator+ microwave and heated to 140° C. for 15 minutes on normal absorption. The contents of the flask were cooled to rt, transferred to a separatory funnel, diluted with EtOAc (30 mL), washed with water (15 mL), followed by saturated NaCl (15 mL), dried over Na₂SO₄, gravity filtered, the solvent was removed in vacuo and the residue was chromatographed on silica gel (EtOAc/Hex, 50:50, v/v to 100% EtOAc, TLC: 100% EtOAc, Rf=0.36) to afford O-Boc (49.0 mg, 32% yield) as a brown semisolid: 1H NMR (500 Hz, CDCl₃) d 8.83 (d, J=1.9 Hz, 1H), 8.77 (s, 1H), 8.68 (dd, J=4.9 Hz, J=1.5 Hz, 1H), 8.61 (d, J=5.1 Hz, 1H), 7.99 (d, J=7.9 Hz, 1H), 7.42 (ddd, J=7.9 Hz, J=4.9 Hz, J=0.4 Hz, 1H), 7.32 (d, J=5.1 Hz, 1H), 4.87 (bs, 1H), 4.08 (d, J=4.7 Hz, 2H), 1.45 (s, 9H).

### Compound O: 3-([3,4′-Bipyridin]-3′-yl)prop-2-yn-1-amine Trihydrochloride

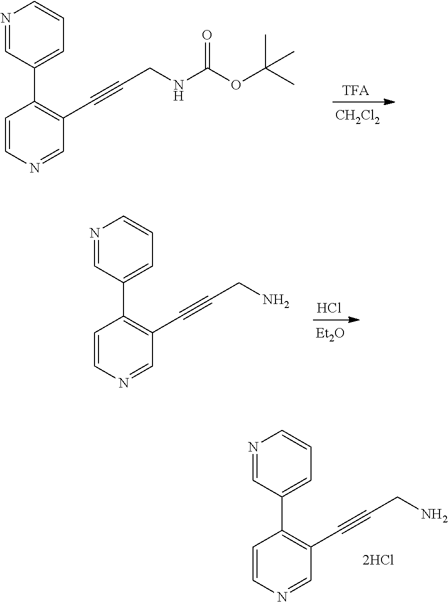

To a solution of O-Boc (49.0 mg, 0.158 mmol) in dichloromethane (0.75 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford 0 (21.0 mg, 42% yield) as a white solid: 1H NMR (500 MHz, D₂O) d 9.14 (d, J=2.1 Hz, 1H), 8.92 (s, 1H), 8.90 (m, 1H), 8.86 (ddd, J=8.2 Hz, J=1.9 Hz, J=1.7 Hz, 1H), 8.75 (d, J=5.4 Hz, 1H), 8.17 (ddd, J=8.2 Hz, J=6.0 Hz, J=0.5 Hz, 1H), 7.79 (dd, J=5.4 Hz, J=0.5 Hz, 1H), 4.02 (s, 2H); 13C NMR (125 MHz, D₂O) d 152.7, 148.7, 148.0, 147.2, 144.0, 143.2, 137.1, 128.4, 126.1, 119.3, 89.7, 81.8, 30.8. HRMS calculated for C13H12N3 [M+H]+, 210.1031, found 210.1032, UPLC (254 nm)>95%.

### Compound P-Boc: tert-Butyl (3-([4,4′-bipyridin]-3′-yl)prop-2-yn-1-yl)carbamate

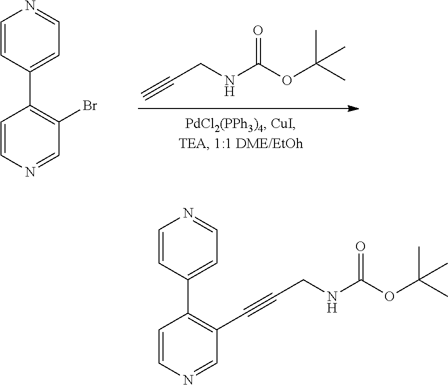 [SMILES: Brc1cnccc1-c1ccncc1 ; C#CCNC(=O)OC(C)(C)C ; CC(C)(C)OC(=O)NCC#Cc1cnccc1-c1ccncc1]

To a 5 mL microwave vial (Biotage) was added 3′-bromo-4, 4′-bipyridine (99.0 mg, 0.421 mmol), tert-butyl prop-2-ynyl-carbamate (68.0 mg, 0.438 mmol), CuI (10.0 mg, 0.0913 mmol) and bis(triphenylphosphine)palladium(II) dichloride (29.0 mg, 0.0413 mmol). The vial was purged with argon for 5 minutes followed by addition of degassed DME/EtOH 50:50 (2.0 mL) and degassed Et3N (175. mL, 1.26 mmol). The vial was capped and placed in a Biotage Initiator+ microwave and heated to 140° C. for 15 minutes on normal absorption. The contents of the flask were cooled to rt, transferred to a separatory funnel, diluted with EtOAc (30 mL), washed with water (15 mL), followed by saturated NaCl (15 mL), dried over Na₂SO₄, gravity filtered, the solvent was removed in vacuo and the residue was chromatographed on silica gel (EtOAc/Hex, 50:50, v/v to 100% EtOAc, TLC: 100% EtOAc, Rf=0.52) to afford P-Boc (39.0 mg, 30% yield) as a yellow semisolid: 1H NMR (500 MHz, CDCl₃) d 8.78 (s, 1H), 8.74 (d, J=5.4 Hz, 2H), 8.62 (d, J=5.1 Hz, 1H), 7.54 (d, J=5.4 Hz, 2H), 7.31 (d, J=5.1 Hz, 1H), 4.97 (bs, 1H), 4.09 (d, J=5.2 Hz, 2H), 1.46 (s, 9H).

### Compound P: 3-([4,4′-Bipyridin]-3′-yl)prop-2-yn-1-amine Trihydrochloride

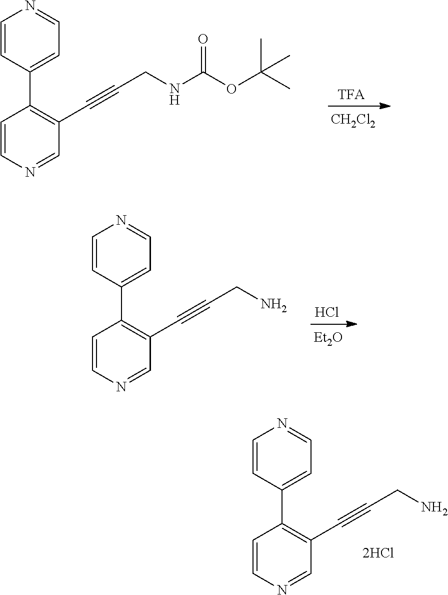

To a solution of P-Boc (39.0 mg, 0.126 mmol) in dichloromethane (0.75 mL), cooled to 0° C. in an external ice water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford P (19.0 mg, 47% yield) as a blue solid. 1H NMR (500 MHz, D₂O) d 9.04 (s, 1H), 8.99 (d, J=6.9 Hz, 2H), 8.86 (d, J=5.7 Hz, 1H), 8.40 (d, J=6.9 Hz, 2H), 7.99 (d, J=5.7 Hz, 1H), 4.04 (s, 2H); 13C NMR (125 MHz, D₂O) d 154.8, 151.2, 151.0, 146.9, 143.2 (2 C), 128.7 (2 C), 126.9, 120.1, 91.3, 80.7, 30.8. HRMS calculated for C13H12N3 [M+H]+, 210.1031, found 210.1049, UPLC (254 nm)>95%.

### Compound Q-Boc: tert-Butyl (3-(4-(pyrimidin-5-yl)pyridin-3-yl)prop-2-yn-1-yl)carbamate

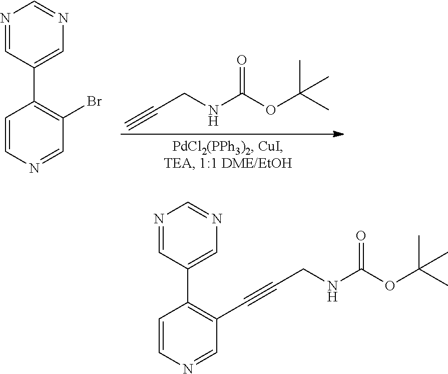

To a 5 mL microwave vial (Biotage) was added 5-(3-bromopyridin-4-yl)pyrimidine (140 mg, 0.593 mmol), tert-butyl prop-2-ynyl-carbamate (93.1 mg, 0.600 mmol), CuI (17.0 mg, 0.0892 mmol) and bis(triphenylphosphine)palladium(II) dichloride (40.0 mg, 0.0570 mmol). The vial was purged with argon for 5 minutes followed by addition of degassed DME/EtOH 50:50 (2.0 mL) and degassed Et3N (250. mL, 1.79 mmol). The vial was capped and placed in a Biotage Initiator+ microwave and heated to 140° C. for 15 minutes on normal absorption. The contents of the flask were cooled to rt, transferred to a separatory funnel, diluted with EtOAc (30 mL), washed with water (15 mL), followed by saturated NaCl (15 mL), dried over Na₂SO₄, gravity filtered, the solvent was removed in vacuo and the residue was chromatographed on silica gel (EtOAc/Hex, 30:70, v/v to 100% EtOAc, TLC: 100% EtOAc, Rf=0.28) to afford Q-Boc (69.0 mg, 37% yield) as a brown semisolid: 1H NMR (500 MHz, CDCl₃) d 9.29 (s, 1H), 9.01 (s, 2H), 8.81 (s, 1H), 8.66 (d, J=5.1 Hz, 1H), 7.33 (d, J=5.1 Hz, 1H), 5.05 (bs, 1H), 4.11 (d, J=4.9 Hz, 2H), 1.46 (s, 9H).

### Compound Q: 3-(4-(Pyrimidin-5-yl)pyridin-3-yl)prop-2-yn-1-amine Trihydrochloride

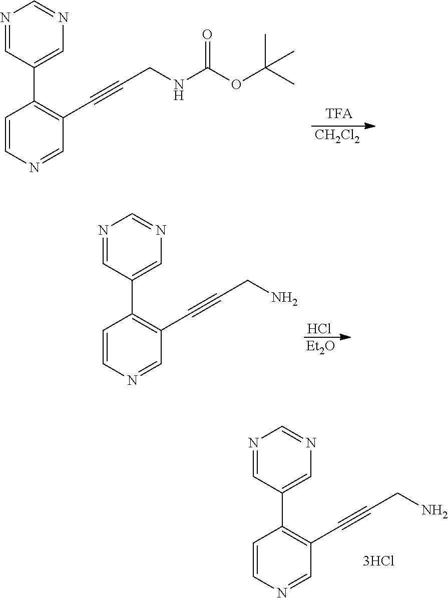

To a solution of Q-Boc (69.0 mg, 0.222 mmol) in dichloromethane (0.75 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford Q (35.0 mg, 49% yield) as a light yellow solid: 1H NMR (500 MHz, D₂O) d 9.31 (s, 1H), 9.21 (s, 2H), 9.07 (s, 1H), 8.87 (d, J=6.0 Hz, 1H), 8.14 (d, J=6.0 Hz, 1H), 4.07 (s, 2H); 13C NMR (125 MHz, D₂O) d 159.8, 158.1 (2 C), 152.9, 148.3, 144.1, 131.4, 128.0, 121.5, 91.5, 80.3, 30.8. HRMS calculated for C12H11N4 [M+H]+, 211.0984, found 211.0986, UPLC (254 nm)=99%.

### Compound R-Boc: tert-butyl ((5-(4-methylpyridin-3-yl)furan-2-yl)methyl)carbamate

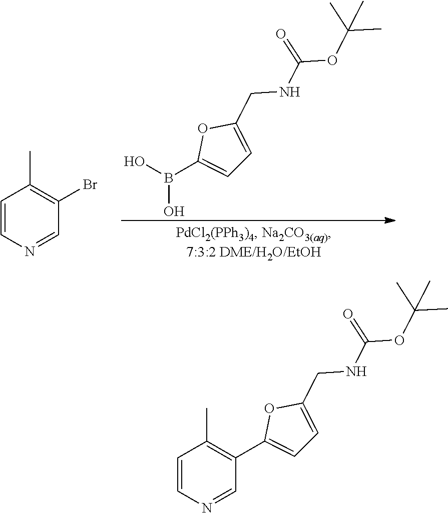

To a Biotage 2.0-5.0 mL microwave tube containing 5-((tert-butoxycarbonyl)aminomethyl)furan-2-boronic acid (134 mg, 0.609 mmol) under a blanket of argon_{(g)} was added degassed DME/H₂O/EtOH 7:3:2 (1.0 mL) followed by trans-dichlorobis(triphenylphosphine)palladium(II) (17 mg, 0.011 mmol) followed by a solution of sodium carbonate (89 mg, 0.83 mmol) in degassed H2O (0.4 mL). The resultant mixture was stirred under argon_{(g)} for 5 minutes followed by the addition of 3-bromo-4-methylpyridine (0.068 mL, 0.61 mmol). The tube was purged with argon_{(g)}, capped and placed in a Biotage Initiator+ microwave and heated to 140° C. for 2 minutes on normal absorption level. The contents of the flask were transferred to an Erlenmeyer flask containing 5 g of anhydrous Na₂SO₄, with the aid of CH₂Cl₂, and subsequently diluted to 50 mL with additional CH₂Cl₂. The Na₂SO₄ was removed by gravity filtration, the solvent was removed in vacuo and the residue was chromatographed on silica gel (EtOAc/Hex, 50:50, v/v Rf=0.29) to afford R-Boc (143. mg, 89% yield) as a red oil: 1H NMR (300 MHz, CDCl₃) d=8.81 (s, 1H), 8.35 (d, J=5.1 Hz, 1H), 7.22-7.02 (m, 1H), 6.52 (d, J=3.2 Hz, 1H), 6.33 (d, J=3.2 Hz, 1H), 5.07 (br. s., 1H), 4.37 (d, J=5.8 Hz, 2H), 2.46 (s, 3H), 1.55-1.22 (m, 9H).

### Compound R: (5-(4-methylpyridin-3-yl)furan-2-yl)methanamine

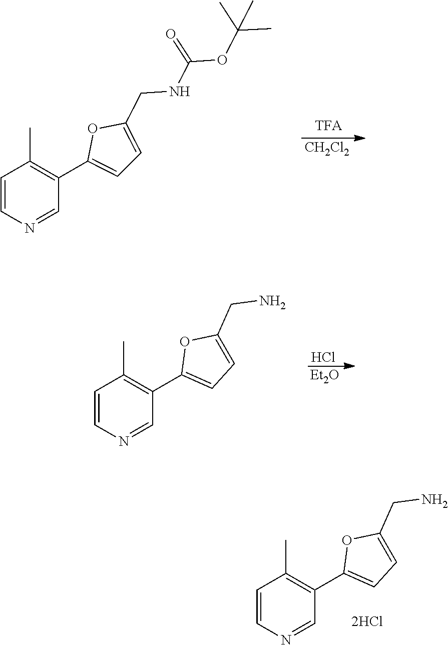 [SMILES: "Et2O" ; Cc1ccncc1-c1ccc(CNC(=O)OC(C)(C)C)o1 ; "CH2Cl2" ; Cc1ccncc1-c1ccc(CN)o1 ; Cl ; Cc1ccncc1-c1ccc(CN)o1]

To a solution of R-Boc (143. mg, 0.50 mmol) in anhydrous dichloromethane (ca. 1.5 mL) was added trifluoroacetic acid (3 mL) and the resultant solution was stirred at ambient temperature for 3 hours. To the mixture was added 2 mL of 1N HCl and the mixture was vigorously stirred for 10 minutes. The mixture was diluted with 20 mL of water, transferred to a separatory funnel and the organics were discarded. The aqueous portion was washed with dichloromethane (2×30 mL), the pH was adjusted to ca. 10 using 10 N NaOH and extracted with dichloromethane (2×35 mL). The combined organics were dried over anhydrous MgSO₄, filtered and the solvent was removed in vacuo. This material was dissolved in 40 mL of ethyl ether and treated with 5 mL of ethereal HCl. The resulting precipitate was collected by centrifugation and dried in vacuo to afford R (12.9 mg, 11% yield) as a white solid: 1H NMR (300 MHz, D₂O) d (s, 1H), 8.33 (dd, J=1.9, 6.0 Hz, 1H), 7.77 (dd, J=2.6, 6.0 Hz, 1H), 6.99-6.77 (m, 1H), 6.74-6.48 (m, 1H), 4.18 (s, 2H), 2.60 (d, J=4.9 Hz, 3H); 13C NMR (75 MHz, D₂O) d 170.1, 151.1, 143.3, 142.6, 133.0, 132.8, 124.4, 110.1, 108.5, 30.76, 17.2.

### Compound S-Boc: tert-butyl ((5-(4-methylpyridin-3-yl)thiophen-2-yl)methyl)carbamate

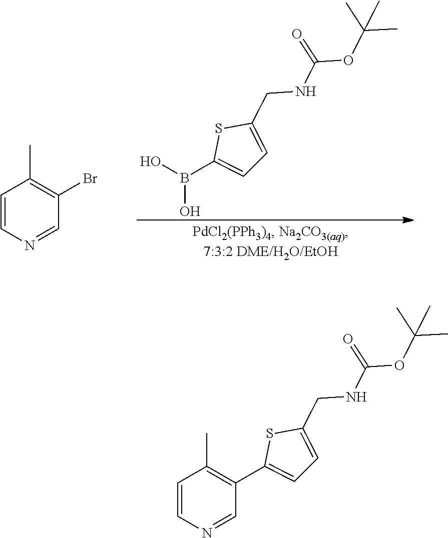

To a Biotage 2.0-5.0 mL microwave tube containing 5-((tert-butoxycarbonyl)aminomethyl)thiophene-2-boronic acid (131 mg, 0.509 mmol) under a blanket of argon_{(g)} was added degassed DME/H₂O/EtOH 7:3:2 (1.0 mL) followed by trans-dichlorobis(triphenylphosphine)palladium(II) (19 mg, 0.027 mmol) followed by a solution of sodium carbonate (82 mg, 0.77 mmol) in degassed H₂O (0.38 mL). The resultant mixture was stirred under argon_{(g)} for 5 minutes followed by the addition of 3-bromo-4-methylpyridine (0.051 mL, 0.46 mmol). The tube was purged with argon_{(g)}, capped and placed in a Biotage Initiator+ microwave and heated to 140° C. for 2 minutes on normal absorption level. The contents of the flask were transferred to an Erlenmeyer flask containing 5 g of anhydrous Na₂SO₄, with the aid of CH₂Cl₂, and subsequently diluted to 50 mL with additional CH₂Cl₂. The Na₂SO₄ was removed by gravity filtration, the solvent was removed in vacuo and the residue was chromatographed on silica gel (EtOAc/Hex, 50:50, v/v Rf=0.36) to afford S-Boc (119. mg, 84% yield) as a red oil: 1H NMR (300 MHz, CDCl₃) d=8.54 (s, 1H), 8.39 (d, J=5.1 Hz, 1H), 7.16 (d, J=4.9 Hz, 1H), 6.96-6.92 (m, 2H), 5.09 (br. S., 1H), 4.49 (d, J=5.8 Hz, 2H), 2.42 (s, 3H), 1.46 (s, 9H).

### Compound S: (5-(4-methylpyridin-3-yl)thiophene-2-yl)methanamine

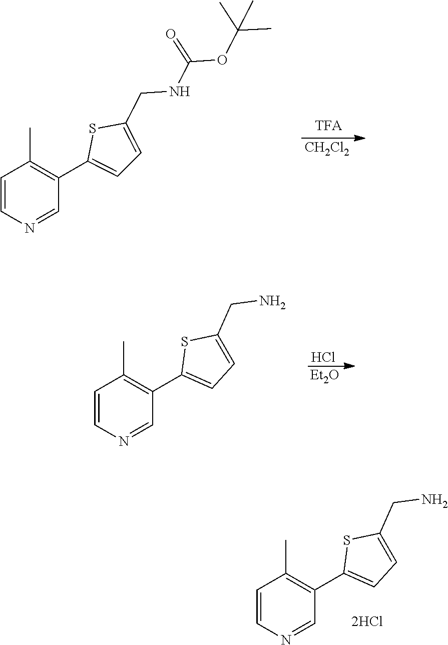 [SMILES: "Et2O" ; Cc1ccncc1-c1ccc(CNC(=O)OC(C)(C)C)s1 ; "CH2Cl2" ; Cc1ccncc1-c1ccc(CN)s1 ; Cl ; Cc1ccncc1-c1ccc(CN)s1]

To a solution of S-Boc (119 mg, 0.39 mmol) in anhydrous dichloromethane (ca. 1.5 mL) was added trifluoroacetic acid (3 mL) and the resultant solution was stirred at ambient temperature for 3 hours. To the mixture was added 2 mL of 1N HCl and the mixture was vigorously stirred for 10 minutes. The mixture was diluted with 20 mL of water, transferred to a separatory funnel and the organics were discarded. The aqueous portion was washed with dichloromethane (2×30 mL), the pH was adjusted to ca. 10 using 10 N NaOH and extracted with dichloromethane (2×35 mL). The combined organics were dried over anhydrous MgSO₄, filtered and the solvent was removed in vacuo. This material was dissolved in 40 mL of ethyl ether and treated with 5 mL of ethereal HCl. The resulting precipitate was collected by centrifugation and dried in vacuo to afford S (26.7 mg, 28% yield) as a white solid: 1H NMR (300 MHz, D₂O) d=8.56 (d, J=6.8 Hz, 1H), 8.39 (br. s., 1H), 7.88-7.65 (m, 1H), 7.25-7.03 (m, 2H), 4.29 (d, J=7.9 Hz, 2H), 2.50 (d, J=10.9 Hz, 3H); 13C NMR (75 MHz, D₂O) d=152.5, 133.7, 132.3, 130.4, 129.2, 127.0, 123.8, 123.7, 122.2, 30.8, 14.8.

### Compound T-Boc: tert-butyl ((5-(4-ethylpyridin-3-yl)furan-2-yl)methyl)carbamate

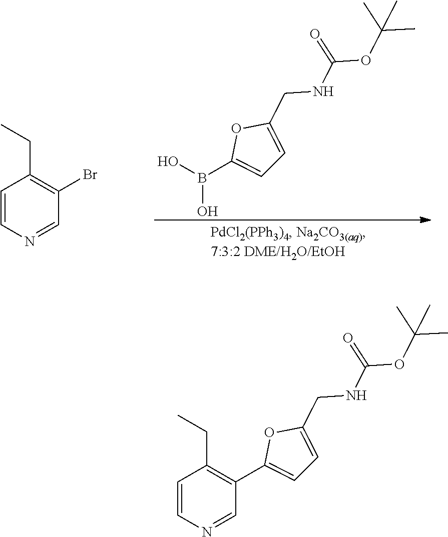

To a 5 mL microwave vial (Biotage) was added 3-bromo-4-ethylpyridine (219 mg, 1.18 mmol), (5-(((tert-butoxycarbonyl)amino)methyl)furan-2-yl)boronic acid (284 mg, 1.18 mmol) and bis(triphenylphosphine)palladium(II) dichloride (9.0 mg, 0.0128 mmol). The vial was purged with argon for 5 minutes followed by addition of degassed DME/EtOH 50:50 (2.0 mL) and degassed Et₃N (175. mL, 1.26 mmol). The vial was capped and placed in a Biotage Initiator+ microwave and heated to 140° C. for 15 minutes on normal absorption. The contents of the flask were cooled to rt, transferred to a separatory funnel, diluted with EtOAc (30 mL), washed with water (15 mL), followed by saturated NaCl (15 mL), dried over Na₂SO₄, gravity filtered, the solvent was removed in vacuo and the residue was chromatographed on silica gel (EtOAc/Hex, 5:95, v/v to EtOAc/Hex, 50:50, v/v, TLC: 50% EtOAc in hexane, Rf=0.36) to afford T-Boc (211 mg, 59% yield) as a brown semisolid: 1H NMR (500 MHz, CDCl₃) d 8.72 (s, 1H), 8.37 (d, J=5.1 Hz, 1H), 7.19 (d, J=5.1 Hz, 1H), 6.52 (d, J=3.3 Hz, 1H), 6.35 (m, 1H), 5.74 (bs, 1H), 2.83 (m, 2H), 1.46 (s, 9H), 1.24 (t, J=7.5 Hz, 3H).

### Compound T: (5-(4-ethylpyridin-3-yl)furan-2-yl)methanamine Dihydrochloride

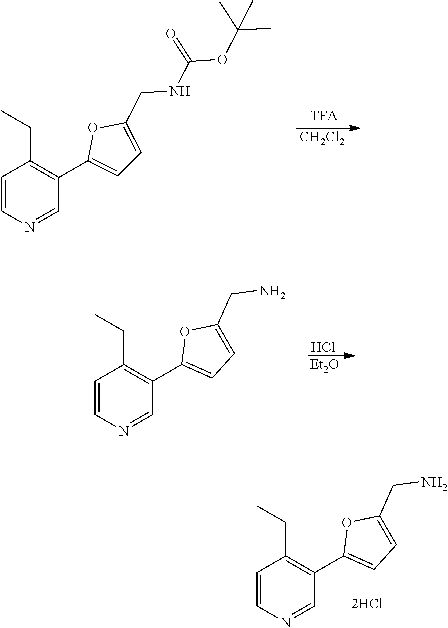 [SMILES: "Et2O" ; CCc1ccncc1-c1ccc(CNC(=O)OC(C)(C)C)o1 ; "CH2Cl2" ; CCc1ccncc1-c1ccc(CN)o1 ; Cl ; CCc1ccncc1-c1ccc(CN)o1]

To a solution of T-Boc (61. mg, 0.202 mmol) in dichloromethane (0.75 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (7.5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford T (39.0 mg, 70% yield) as a yellow solid. 1H NMR (500 MHz, D₂O) d 8.93 (s, 1H), 8.54 (d, J=6.0 Hz, 1H), 7.91 (d, J=6.0 Hz, 1H), 7.01 (d, J=3.5 Hz, 1H), 6.78 (d, J=3.5 Hz, 1H), 4.35 (s, 2H), 3.09 (q, J=7.5 Hz, 2H), 1.32 (t, J=7.5 Hz, 3H); 13C NMR (125 MHz, D₂O) d 155.5, 143.3, 142.9, 134.6, 134.5, 123.7, 121.9, 109.5, 108.4, 30.8, 22.5, 7.1. HRMS calculated for C12H16N2O [M+H]+, 203.1179, found 203.1184, UPLC (254 nm)>90%.

### Compound U-Boc: tert-butyl ((5-(4-ethylpyridin-3-yl)thiophene-2-yl)methyl)carbamate

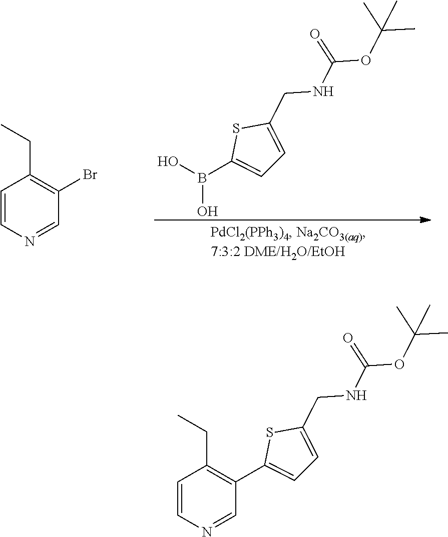

To a 5 mL microwave vial (Biotage) was added 3-bromo-4-ethylpyridine (107 mg, 0.575 mmol), (5-(((tert-butoxycarbonyl)amino)methyl)thiophen-2-yl)boronic acid (147 mg, 0.572 mmol) and bis(triphenylphosphine)palladium(II) dichloride (8.0 mg, 0.0114 mmol). The vial was purged with argon for 5 minutes followed by adding the degassed solvent of DME/H₂O/EtOH (7:3:2, v:v:v, 2.0 mL) and degassed 2 M Na₂CO₃ (0.75 mL). The vial was capped and the stirring slurry was heated at 140° C. by microwave irradiation on normal absorption level for 5 minutes, cooled to rt, diluted with water (20 mL), extracted with dichloromethane (2×20 mL), dried over Na₂SO₄, gravity filtered and the solvent was removed in vacuo to afford the crude material which was purified by flash chromatography using a gradient elution (EtOAc/Hex, 5:95, v/v to EtOAc/Hex, 50:50, v/v, TLC: 50% EtOAc in hexane, Rf=0.33) to afford the product U-Boc (69.0 mg, 38% yield) as a yellow syrup: 1H NMR (500 MHz, CDCl₃) d 1H NMR (500 MHz, CDCl3) d 8.47 (s, 1H), 8.43 (m, 1H), 7.24 (d, J=5.1 Hz, 1H), 6.96 (m, 1H), 6.90 (d, J=3.5 Hz, 1H), 5.45 (bs, 1H), 4.50 (m, 2H), 2.77 (q, J=7.6 Hz, 2H), 1.48 (s, 9H), 1.20 (t, J=7.6 Hz, 3H).

### Compound U: (5-(4-ethylpyridin-3-yl)thiophene-2-yl)methanamine Dihydrochloride

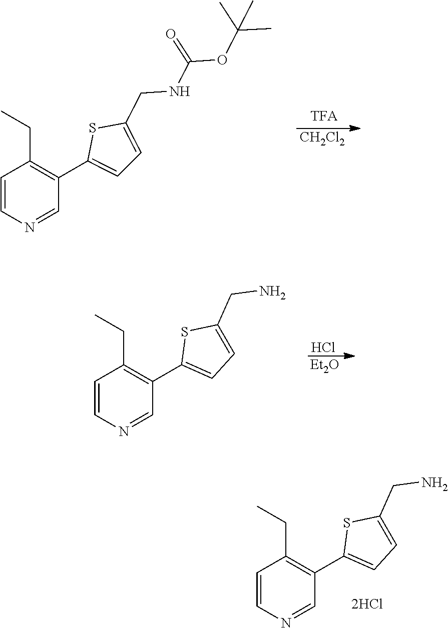 [SMILES: "Et2O" ; CCc1ccncc1-c1ccc(CNC(=O)OC(C)(C)C)s1 ; "CH2Cl2" ; CCc1ccncc1-c1ccc(CN)s1 ; Cl ; CCc1ccncc1-c1ccc(CN)s1]

To a solution of U-Boc (69.0 mg, 0.217 mmol) in dichloromethane (0.5 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×20 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford U (40.5 mg, 64% yield) as a yellow solid. 1H NMR (500 MHz, D₂O) d 8.45 (s, 1H), 8.40 (d, J=5.4 Hz, 1H), 7.47 (d, J=5.4 Hz, 1H), 7.26 (d, J=3.6 Hz, 1H), 7.12 (d, J=3.6 Hz, 1H), 4.42 (s, 2H), 2.78 (q, J=7.6 Hz, 2H), 1.14 (t, J=7.6 Hz, 3H); 13C NMR (125 MHz, D₂O) d 147.7, 141.5, 140.6, 132.8, 128.4, 123.4, 123.3, 121.9, 117.8, 30.8, 19.1, 6.9. HRMS calculated for C12H15N2S [M+H]+, 219.0950, found 219.0961, UPLC (254 nm)>95%.

### Compound V-Boc: tert-butyl ((5-(4-propylpyridin-3-yl)furan-2-yl)methyl)carbamate

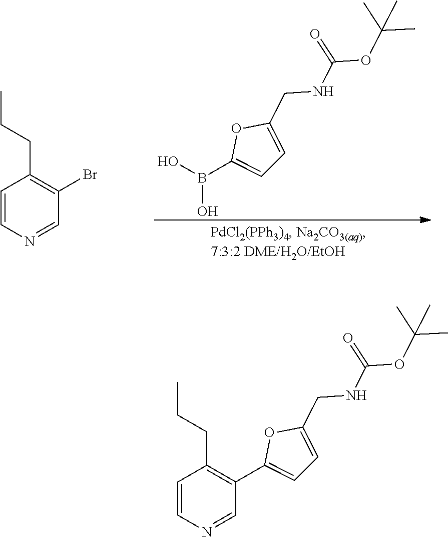

To a 5 mL microwave vial (Biotage) was added 3-bromo-4-propylpyridine (167 mg, 0.835 mmol), (5-(((tert-butoxycarbonyl)amino)methyl)furan-2-yl)boronic acid (202 mg, 0.838 mmol) and bis(triphenylphosphine)palladium(II) dichloride (29.0 mg, 0.0413 mmol). The vial was purged with argon for 5 minutes followed by adding the degassed solvent of DME/H₂O/EtOH (7:3:2, v:v:v, 2.0 mL) and degassed 2 M Na₂CO₃ (0.7 mL). The vial was capped and placed in a Biotage Initiator+ microwave and heated to 140° C. for 5 minutes on normal absorption. The contents of the flask were cooled to rt, transferred to a separatory funnel, diluted with EtOAc (30 mL), washed with water (15 mL), followed by saturated NaCl (15 mL), dried over Na₂SO₄, gravity filtered, the solvent was removed in vacuo and the residue was chromatographed on silica gel (EtOAc/Hex, 5:95, v/v to EtOAc/Hex, 50:50, v/v, TLC: EtOAc/Hex, 50:50, v/v, Rf=0.30) to afford V-Boc (97.0 mg, 37% yield) as a yellow semisolid: 1H NMR (500 MHz, CDCl₃) d 8.84 (s, 1H), 8.39 (d, J=5.1 Hz, 1H), 7.13 (d, J=5.1 Hz, 1H), 6.48 (d, J=2.9 Hz, 1H), 6.34 (m, 1H), 5.29 (bs, 1H), 4.38 (d, J=5.4 Hz, 2H), 2.76 (t, J=7.8 Hz, 2H), 1.62 (m, 2H), 1.46 (s, 9H), 0.98 (t, J=7.4 Hz, 3H).

### Compound V: (5-(4-propylpyridin-3-yl)furan-2-yl)methanamine Dihydrochloride

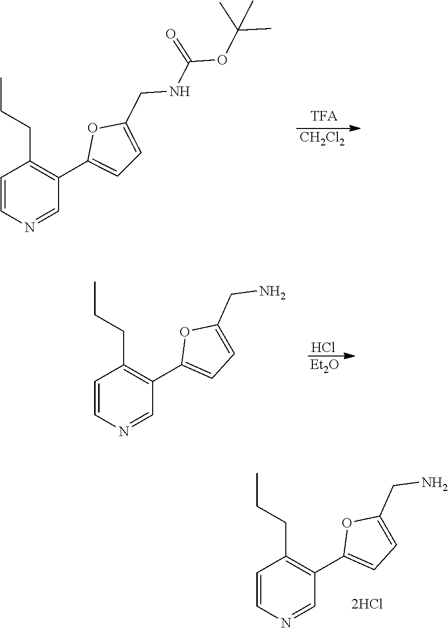 [SMILES: "Et2O" ; CCCc1ccncc1-c1ccc(CNC(=O)OC(C)(C)C)o1 ; "CH2Cl2" ; CCCc1ccncc1-c1ccc(CN)o1 ; Cl ; CCCc1ccncc1-c1ccc(CN)o1]

To a solution of V-Boc (97. mg, 0.31 mmol) in dichloromethane (0.75 mL), cooled to 0° C. in an external ice-water bath, was added trifluoroacetic acid (0.75 mL). The ice bath was removed and the resultant solution was stirred at ambient temperature for 2 hours. The reaction mixture was subsequently chilled in an external ice-water bath followed by dropwise adding saturated sodium carbonate (7.5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford V (54.0 mg, 81% yield) as a brown solid: 1H NMR (500 MHz, D₂O) d 8.96 (s, 1H), 8.54 (d, J=6.1 Hz, 1H), 7.97 (d, J=6.1 Hz, 1H), 7.01 (d, J=3.6 Hz, 1H), 6.78 (d, J=3.6 Hz, 1H), 4.34 (s, 2H), 3.08 (t, J=7.7 Hz, 2H), 1.73 (m, 2H), 0.98 (t, J=7.4 Hz, 3H); 13C NMR (125 MHz, D₂O) d 155.4, 143.5, 142.5, 133.9, 133.3, 124.2, 123.2, 109.7, 108.4, 31.1, 30.8, 16.6, 8.1. H RMS calculated for C13H17N2O [M+H]+, 217.1341, found 213.1346, UPLC (254 nm)>95%.

### Compound W-Boc: tert-butyl ((5-(4-propylpyridin-3-yl)thiophene-2-yl)methyl)carbamate

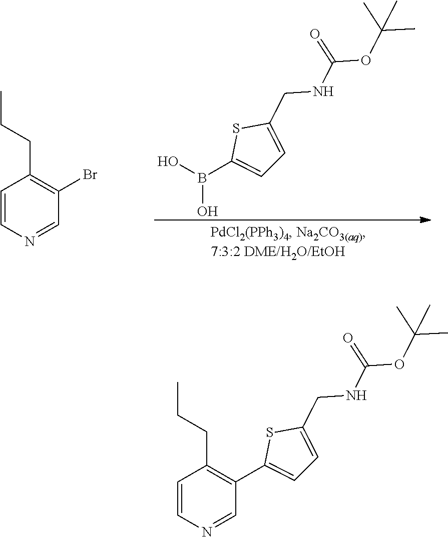

To a 5 mL microwave vial (Biotage) was added 3-bromo-4-propylpyridine (153 mg, 0.765 mmol), (5-(((tert-butoxycarbonyl)amino)methyl)thiophen-2-yl)boronic acid (196 mg, 0.762 mmol) and bis(triphenylphosphine)palladium(II) dichloride (27.0 mg, 0.0385 mmol). The vial was purged with argon for 5 minutes followed by adding the degassed solvent of DME/H₂O/EtOH (7:3:2, v:v:v, 2.0 mL) and degassed 2 M Na₂CO₃ (0.7 mL). The vial was capped and placed in a Biotage Initiator+ microwave and heated to 140° C. for 5 minutes on normal absorption. The contents of the flask were cooled to rt, transferred to a separatory funnel, diluted with EtOAc (30 mL), washed with water (15 mL), followed by saturated NaCl (15 mL), dried over Na₂SO₄, gravity filtered, the solvent was removed in vacuo and the residue was chromatographed on silica gel (EtOAc/Hex, 5:95, v/v to EtOAc/Hex, 50:50, v/v, TLC: EtOAc/Hex, 50:50, v/v, Rf=0.30) to afford W-Boc (95.0 mg, 38% yield) as a yellow semisolid: 1H NMR (500 MHz, CDCl₃) d 8.50 (s, 1H), 8.44 (d, J=5.1 Hz, 1H), 7.18 (d, J=5.1 Hz, 1H), 6.96 (m, 1H), 6.88 (d, J=3.5 Hz, 1H), 5.25 (bs, 1H), 4.51 (m, 2H), 2.70 (t, J=7.8 Hz, 2H), 1.59 (m, 2H), 1.47 (s, 9H), 0.93 (t, J=7.3 Hz, 3H).

### Compound W: (5-(4-propylpyridin-3-yl)thiophene-2-yl)methanamine Dihydrochloride

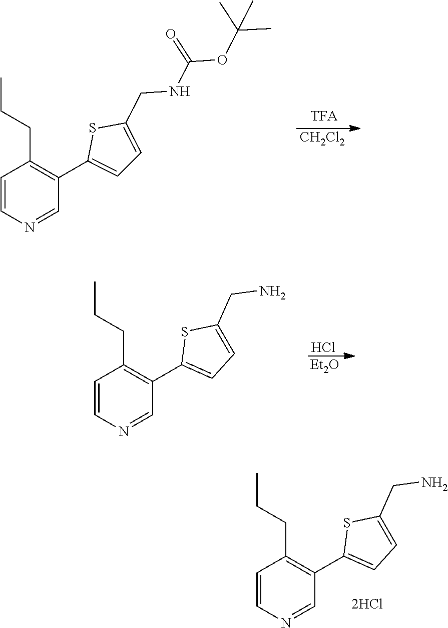 [SMILES: "Et2O" ; CCCc1ccncc1-c1ccc(CNC(=O)OC(C)(C)C)s1 ; "CH2Cl2" ; CCCc1ccncc1-c1ccc(CN)s1 ; Cl ; CCCc1ccncc1-c1ccc(CN)s1]

To a solution of W-Boc (95. mg, 0.29 mmol) in dichloromethane (0.75 mL), cooled to 0° C. in an external ice-water bath, was added trifluoroacetic acid (0.75 mL). The ice bath was removed and the resultant solution was stirred at ambient temperature for 2 hours. The reaction mixture was subsequently chilled in an external ice-water bath followed by dropwise adding saturated sodium carbonate (7.5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford W (51.0 mg, 77% yield) as an off-white solid: 1H NMR (500 MHz, D₂O) d 8.65 (s, 1H), 8.56 (d, J=6.0 Hz, 1H), 7.86 (d, J=6.0 Hz, 1H), 7.31 (d, J=3.7 Hz, 1H), 7.23 (d, J=3.7 Hz, 1H), 4.45 (s, 2H), 2.91 (t, J=7.7 Hz, 2H), 1.62 (m, 2H), 0.88 (t, J=7.4 Hz, 3H); 13C NMR (125 MHz, D₂O) d 154.4, 136.1, 134.6, 130.0, 129.9, 126.2, 123.5, 123.3, 120.5, 30.8, 28.6, 16.1, 6.4. HRMS calculated for C13H17N2S [M+H]+, 233.1112, found 233.1120, UPLC (254 nm)>95%.

### Compound X-Boc: tert-butyl ((5-(4-phenylpyridin-3-yl)furan-2-yl)methyl)carbamate

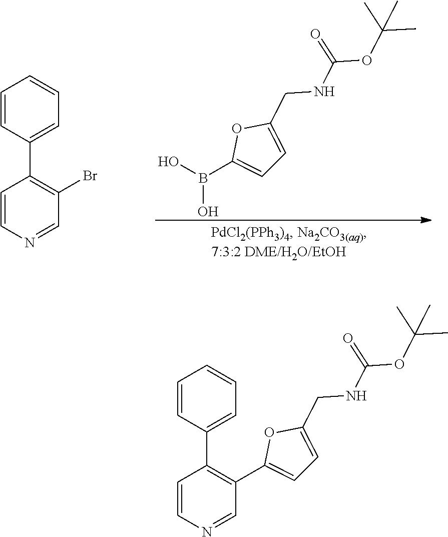 [SMILES: Brc1cnccc1-c1ccccc1 ; CC(C)(C)OC(=O)NCc1ccc(B(O)O)o1 ; CC(C)(C)OC(=O)NCc1ccc(-c2cnccc2-c2ccccc2)o1]

To a Biotage 2.0-5.0 mL microwave tube containing 5-((tert-butoxycarbonyl)aminomethyl)furan-2-boronic acid (205. mg, 0.849 mmol) under a blanket of argon_{(g)} was added degassed DME/H₂O/EtOH 7:3:2 (1.50 mL) followed by trans-dichlorobis(triphenylphosphine)palladium(II) (16. mg, 0.023 mmol) followed by a solution of sodium carbonate (132. mg, 1.25 mmol) in degassed H₂O (0.65 mL). The resultant mixture was stirred under argon_{(g)} for 5 minutes followed by the addition of a solution of 3-bromo-4-phenylpyridine (221 mg, 0.946 mmol) in degassed DME/H₂O/EtOH 7:3:2 (1.00 mL). The tube was purged with argon_{(g)}, capped and placed in a Biotage Initiator+ microwave and heated to 140° C. for 4 minutes on normal absorption level. The contents of the flask were transferred to an Erlenmeyer flask containing 5 g of anhydrous Na₂SO₄, with the aid of CH₂Cl₂, and subsequently diluted to 50 mL with additional CH₂Cl₂. The Na₂SO₄ was removed by gravity filtration, the solvent was removed in vacuo and the residue was chromatographed on silica gel (EtOAc/Hex, 25:75, v/v Rf=0.13-0.21) to afford X-Boc (118. mg, 40% yield) as a colorless oil: 1H NMR (300 MHz, CDCl₃) d=8.80 (br. s., 1H), 8.37 (br. s., 1H), 7.30-7.21 (m, 5H), 7.18-7.02 (m, 5H), 5.96 (d, J=3.0 Hz, 1H), 5.65 (d, J=3.0 Hz, 1H), 4.64 (br. s., 1H), 4.05 (d, J=5.3 Hz, 2H), 1.41-1.16 (m, 9H).

### Compound X: (5-(4-phenylpyridin-3-yl)furan-2-yl)methanamine

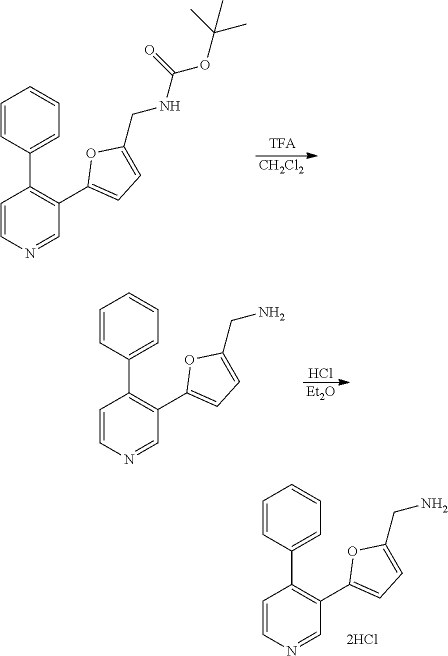 [SMILES: "Et2O" ; CC(C)(C)OC(=O)NCc1ccc(-c2cnccc2-c2ccccc2)o1 ; "CH2Cl2" ; NCc1ccc(-c2cnccc2-c2ccccc2)o1 ; Cl ; NCc1ccc(-c2cnccc2-c2ccccc2)o1]

To a solution of X-Boc (118 mg, 0.336 mmol) in anhydrous dichloromethane (ca. 4 mL) was added trifluoroacetic acid (4 mL) and the resultant solution was stirred at ambient temperature for 2.5 hours. The mixture was transferred to a separatory funnel followed by 5 mL of 1N HCl, 10 mL of H₂O and 45 mL of dichloromethane. The organics were discarded and the aqueous portion was washed with dichloromethane (45 mL), the pH was adjusted to ca. 10 using 10 N NaOH and extracted with dichloromethane (2×50 mL). The combined organics were dried over anhydrous MgSO₄, filtered and the solvent was removed in vacuo. This material was dissolved in 40 mL of diethyl ether and treated with 5 mL of ethereal HCl. The resulting precipitate was collected by centrifugation and dried in vacuo to afford X (28.2 mg, 29% yield) as a white solid: 1H NMR (300 MHz, CDCl₃) d=8.90 (br. s., 1H), 8.49 (br. s., 1H), 7.74 (br. s., 1H), 7.53-7.29 (m, 3H), 7.26 (br. s., 2H), 6.43-6.16 (m, 1H), 5.92 (br. s., 1H), 4.03 (s, 2H).

### Compound Y-Boc: tert-butyl ((5-(4-phenylpyridin-3-yl)thiophene-2-yl)methyl)carbamate

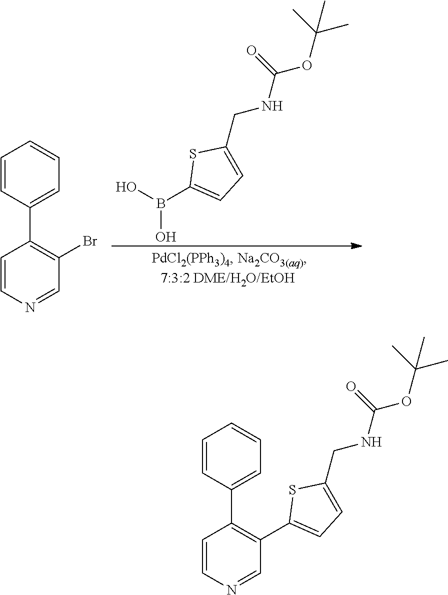 [SMILES: Brc1cnccc1-c1ccccc1 ; CC(C)(C)OC(=O)NCc1ccc(B(O)O)s1 ; CC(C)(C)OC(=O)NCc1ccc(-c2cnccc2-c2ccccc2)s1]

To a Biotage 2.0-5.0 mL microwave tube containing 5-((tert-butoxycarbonyl)aminomethyl)thiophene-2-boronic acid (245 mg, 0.971 mmol) under a blanket of argon_{(g)} was added degassed DME/H₂O/EtOH 7:3:2 (0.75 mL) followed by trans-dichlorobis(triphenylphosphine)palladium(II) (22. mg, 0.031 mmol) followed by a solution of sodium carbonate (160 mg, 1.51 mmol) in degassed H₂O (0.75 mL). The resultant mixture was stirred under argon_{(g)} for 5 minutes followed by the addition of a solution of 3-bromo-4-phenylpyridine (250 mg, 1.07 mmol) in degassed DME/H₂O/EtOH 7:3:2 (1.50 mL). The tube was purged with argon_{(g)}, capped and placed in a Biotage Initiator+ microwave and heated to 140° C. for 2 minutes on normal absorption level. The contents of the flask were transferred to an Erlenmeyer flask containing 5 g of anhydrous Na₂SO₄, with the aid of CH₂Cl₂, and subsequently diluted to 50 mL with additional CH₂Cl₂. The Na₂SO₄ was removed by gravity filtration, the solvent was removed in vacuo and the residue was chromatographed on silica gel (EtOAc/Hex, 25:75, v/v Rf=0.13) to afford Y-Boc (193. mg, 52% yield) as a yellow oil: 1H NMR (300 MHz, CDCl₃) d 8.70 (br. s., 1H), 8.55 (d, J=4.3 Hz, 1H), 7.45-7.18 (m, 6H), 6.76 (d, J=3.4 Hz, 1H), 6.62 (d, J=3.6 Hz, 1H), 5.23 (br. s., 1H), 4.52-4.20 (m, 2H), 1.45 (s, 9H).

### Compound Y: (5-(4-phenylpyridin-3-yl)thiophene-2-yl)methanamine

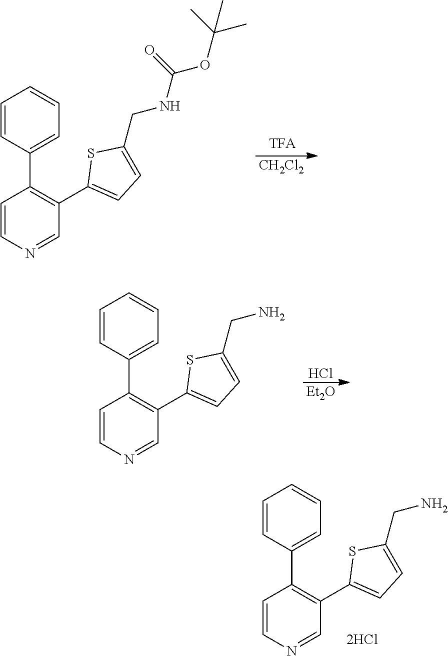 [SMILES: "Et2O" ; CC(C)(C)OC(=O)NCc1ccc(-c2cnccc2-c2ccccc2)s1 ; "CH2Cl2" ; NCc1ccc(-c2cnccc2-c2ccccc2)s1 ; Cl ; NCc1ccc(-c2cnccc2-c2ccccc2)s1]

To a solution of Y-Boc (193 mg, 0.526 mmol) in anhydrous dichloromethane (ca. 5 mL) was added trifluoroacetic acid (3 mL) and the resultant solution was warmed to reflux and stirred until complete conversion was determined by TLC analysis (1 h). The mixture cooled to rt, transferred to a separatory funnel followed by 10 mL of H₂O, 10 mL of 1N HCl and dichloromethane (40 mL). Thor organics were discarded, the aqueous layer was washed with 40 mL of dichloromethane. The pH of the aqueous layer was adjusted to ca. 10 using 10 N NaOH and extracted with dichloromethane (2×50 mL). The combined organics were dried over anhydrous MgSO₄, filtered and the solvent was removed in vacuo. The residue was chromatographed on 10 g of silica gel (10% CH₃OH in dichloromethane, Rf=0.119). This material was dissolved in 40 mL of ethyl ether and treated with 3 mL of ethereal HCl. The resulting precipitate was collected by centrifugation and dried in vacuo to afford Y (87.7 mg, 55% yield) as a white solid: 1H NMR (300 MHz, D₂O) d=8.71 (s, 1H), 8.53 (d, J=6.0 Hz, 1H), 7.75 (d, J=6.0 Hz, 1H), 7.42-7.09 (m, 5H), 6.97 (d, J=3.8 Hz, 1H), 6.89 (d, J=3.8 Hz, 1H), 4.17-4.05 (m, 2H).

### Compound Z-Boc: tert-butyl ((5-(4-methoxypyridin-3-yl)furan-2-yl)methyl)carbamate

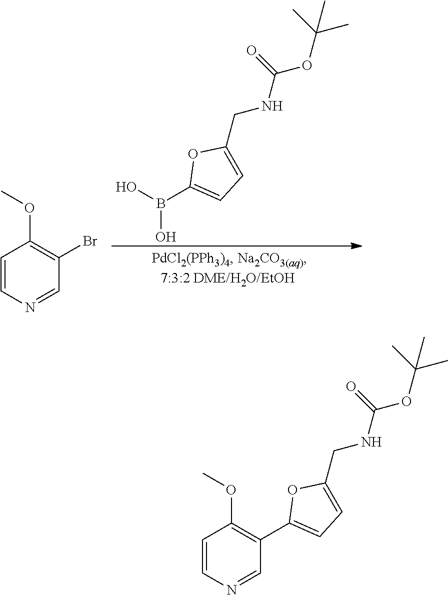

To a 5 mL microwave vial (Biotage) was added 3-bromo-4-methoxypyridine (131 mg, 0.697 mmol), (5-(((tert-butoxycarbonyl)amino)methyl)furan-2-yl)boronic acid (168 mg, 0.698 mmol) and bis(triphenylphosphine)palladium(II) dichloride (28.0 mg, 0.0399 mmol). The vial was purged with argon for 5 minutes followed by adding the degassed solvent of DME/H₂O/EtOH (7:3:2, v:v:v, 2.0 mL) and degassed 2 M Na₂CO₃ (0.7 mL). The vial was capped and placed in a Biotage Initiator+ microwave and heated to 140° C. for 15 minutes on normal absorption. The contents of the flask were cooled to rt, transferred to a separatory funnel, diluted with EtOAc (30 mL), washed with water (15 mL), followed by saturated NaCl (15 mL), dried over Na₂SO₄, gravity filtered, the solvent was removed in vacuo and the residue was chromatographed on silica gel (EtOAc/Hex, 5:95, v/v to EtOAc/Hex, 50:50, v/v, TLC: EtOAc/Hex, 50:50, v/v, Rf=0.30) to afford Z-Boc (162 mg, 76% yield) as a reddish semisolid: 1H NMR (500 MHz, CDCl₃) d 8.92 (s, 1H), 8.38 (d, J=5.8 Hz, 1H), 6.84-6.86 (m 2H), 6.32 (m, 1H), 5.07 (bs, 1H), 4.38 (d, J=5.4 Hz, 2H), 3.98 (s, 3H), 1.47 (s, 9H).

### Compound Z: (5-(4-methoxypyridin-3-yl)furan-2-yl)methanamine Dihydrochloride

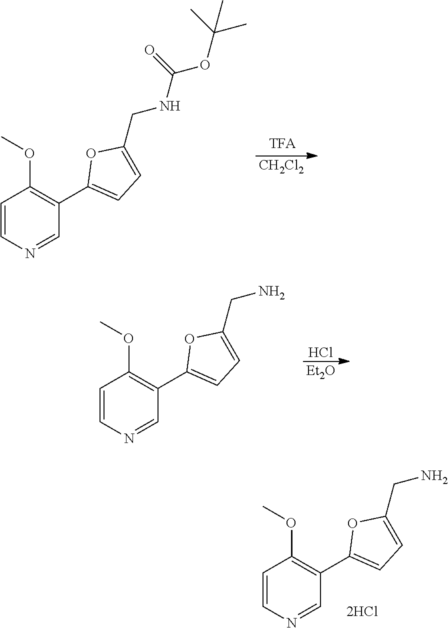 [SMILES: "Et2O" ; COc1ccncc1-c1ccc(CNC(=O)OC(C)(C)C)o1 ; "CH2Cl2" ; COc1ccncc1-c1ccc(CN)o1 ; Cl ; COc1ccncc1-c1ccc(CN)o1]

To a solution of Z-Boc (162 mg, 0.532 mmol) in dichloromethane (0.75 mL), cooled to 0° C. in an external ice-water bath, was added trifluoroacetic acid (0.75 mL). The ice bath was removed and the resultant solution was stirred at ambient temperature for 2 hours. The reaction mixture was subsequently chilled in an external ice-water bath followed by dropwise adding saturated sodium carbonate (7.5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford Z (72.0 mg, 49% yield) as a brown solid: 1H NMR (500 MHz, D₂O) d 8.76 (s, 1H), 8.37 (d, J=6.3 Hz, 1H), 7.26 (d, J=6.3 Hz, 1H), 7.05 (d, J=3.4 Hz, 1H), 6.67 (d, J=3.4 Hz, 1H), 4.29 (s, 2H), 4.07 (s, 3H); 13C NMR (125 MHz, D₂O) d 158.7, 142.2, 141.5, 141.2, 137.6, 111.8, 108.6, 108.2, 103.4, 51.6, 30.8. HRMS calculated for C11H13N2O2 [M+H]+, 205.0977, found 205.0977, UPLC (254 nm)>95%.

### Compound AA-Boc: tert-butyl ((5-(4-methoxypyridin-3-yl)thiophene-2-yl)methyl)carbamate

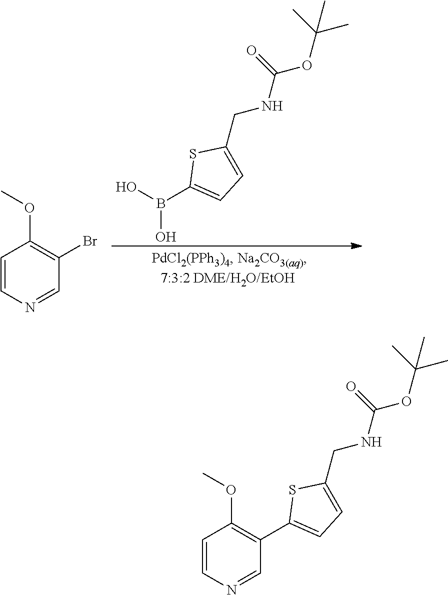

To a 5 mL microwave vial (Biotage) was added 3-bromo-4-methoxypyridine (173 mg, 0.918 mmol), (5-(((tert-butoxycarbonyl)amino)methyl)thiophen-2-yl)boronic acid (235 mg, 0.916 mmol) and bis(triphenylphosphine)palladium(II) dichloride (35.0 mg, 0.0499 mmol). The vial was purged with argon for 5 minutes followed by adding the degassed solvent of DME/H₂O/EtOH (7:3:2, v:v:v, 2.0 mL) and degassed 2 M Na₂CO₃ (0.7 mL). The vial was capped and placed in a Biotage Initiator+ microwave and heated to 140° C. for 5 minutes on normal absorption. The contents of the flask were cooled to rt, transferred to a separatory funnel, diluted with EtOAc (30 mL), washed with water (15 mL), followed by saturated NaCl (15 mL), dried over Na₂SO₄, gravity filtered, the solvent was removed in vacuo and the residue was chromatographed on silica gel (EtOAc/Hex, 5:95, v/v to EtOAc/Hex, 50:50, v/v, TLC: EtOAc/Hex, 50:50, v/v, Rf=0.32) to afford AA-Boc (244 mg, 83% yield) as a light yellow syrup: 1H NMR (500 MHz, CDCl₃) d 8.67 (s, 1H), 8.37 (d, J=5.7 Hz, 1H), 7.32 (d, J=3.7 Hz, 1H), 6.93 (m, 1H), 6.86 (d, J=5.7 Hz, 1H), 5.32 (bs, 1H), 4.49 (m, 2H), 3.94 (s, 3H), 1.47 (s, 9H).

### Compound AA: (5-(4-methoxypyridin-3-yl)thiophene-2-yl)methanamine Dihydrochloride

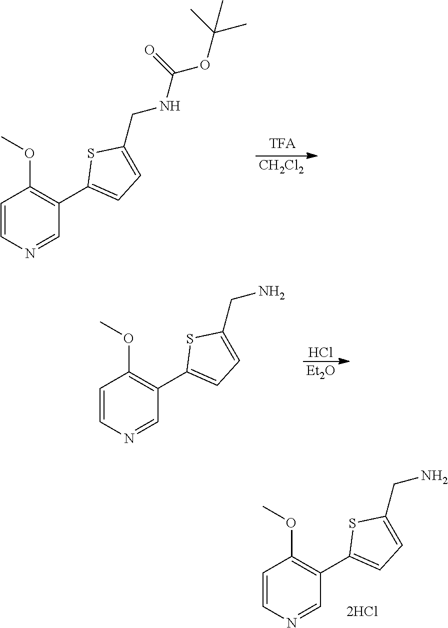 [SMILES: "Et2O" ; COc1ccncc1-c1ccc(CNC(=O)OC(C)(C)C)s1 ; "CH2Cl2" ; COc1ccncc1-c1ccc(CN)s1 ; Cl ; COc1ccncc1-c1ccc(CN)s1]

To a solution of AA-Boc (244. mg, 0.762 mmol) in dichloromethane (0.75 mL), cooled to 0° C. in an external ice-water bath, was added trifluoroacetic acid (0.75 mL). The ice bath was removed and the resultant solution was stirred at ambient temperature for 2 hours. The reaction mixture was subsequently chilled in an external ice-water bath followed by dropwise adding saturated sodium carbonate (7.5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford AA (139. mg, 62% yield) as an off-white solid: 1H NMR (500 MHz, D₂O) d 8.43 (s, 1H), 8.20 (d, J=5.9 Hz, 1H), 7.33 (d, J=3.8 Hz, 1H), 7.09 (d, J=3.8 Hz, 1H), 6.98 (d, J=5.9 Hz, 1H), 4.25 (s, 2H), 3.88 (s, 3H); 13C NMR (125 MHz, D₂O) d 154.5, 141.9, 140.0, 130.3, 128.9, 121.2, 119.4, 111.9, 100.3, 48.4, 30.8. HRMS calculated for C11H13N2O [M+H]+, 221.0749, found 221.0754, UPLC (254 nm)>95%.

### Compound AB-Boc: tert-Butyl ((5-(4-chloropyridin-3-yl)thiophene-2-yl)methyl)carbamate

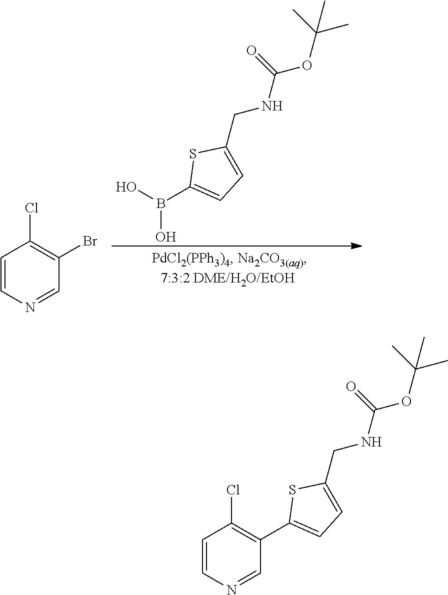 [SMILES: Clc1ccncc1Br ; CC(C)(C)OC(=O)NCc1ccc(B(O)O)s1 ; CC(C)(C)OC(=O)NCc1ccc(-c2cnccc2Cl)s1]

To a 5 mL microwave vial (Biotage) was added 3-bromo4-chloropyridine (236 mg, 1.23 mmol), (5-(((tert-butoxycarbonyl)amino)methyl)thiophen-2-yl)boronic acid (315 mg, 1.23 mmol) and bis(triphenylphosphine)palladium(II) dichloride (49.0 mg, 0.0698 mmol). The vial was purged with argon for 5 minutes followed by adding the degassed solvent of DME/H₂O/EtOH (7:3:2, v:v:v, 2.0 mL) and degassed 2 M Na₂CO₃ (0.7 mL). The vial was capped and placed in a Biotage Initiator+ microwave and heated to 140° C. for 5 minutes on normal absorption. The contents of the flask were cooled to rt, transferred to a separatory funnel, diluted with water (20 mL), extracted with CH₂Cl₂ (2×20 mL), dried over Na₂SO₄, gravity filtered, the solvent was removed in vacuo and the residue was chromatographed on silica gel using a gradient elution (EtOAc/Hex, 20:80, v/v to EtOAc/Hex, 60:40, v/v, TLC: EtOAc/Hex, 50:50, v/v, Rf=0.55) to afford AB-Boc (281 mg, 70% yield) as a light yellow syrup: 1H NMR (500 MHz, CDCl₃) d 8.65 (s, 1H), 8.37 (d, J=5.4 Hz, 1H), 7.37 (d, J=5.4 Hz, 1H), 7.24 (d, J=3.7 Hz, 1H), 6.97 (m, 1H), 5.73 (bs, 1H), 4.51 (m, 2H), 1.47 (s, 9H).

### Compound AB: (5-(4-chloropyridin-3-yl)thiophene-2-yl)methanamine Dihydrochloride

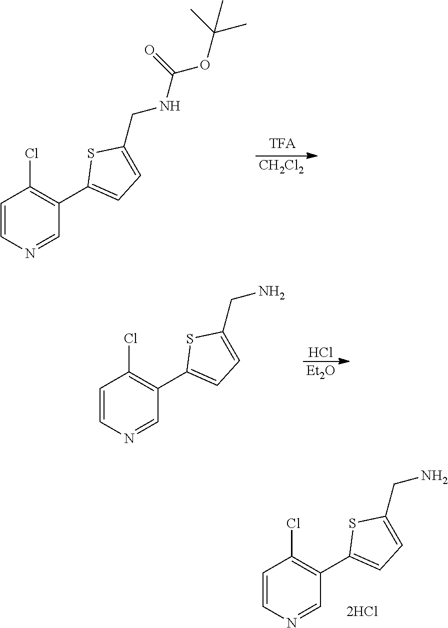 [SMILES: "Et2O" ; CC(C)(C)OC(=O)NCc1ccc(-c2cnccc2Cl)s1 ; "CH2Cl2" ; NCc1ccc(-c2cnccc2Cl)s1 ; Cl ; NCc1ccc(-c2cnccc2Cl)s1]

To a solution of AB-Boc (49.0 mg, 0.151 mmol) in dichloromethane (0.5 mL), cooled to 0° C. in an external ice water bath, was added trifluoroacetic acid (0.5 mL). The ice bath was removed and the resultant solution was stirred at ambient temperature for 3 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford AB (30.0 mg, 67% yield) as an off-white solid: 1H NMR (500 MHz, D₂O) d 8.96 (s, 1H), 8.63 (dd, J=6.3 Hz, J=0.8 Hz, 1H), 8.14 (d, J=6.3 Hz, 1H), 7.55 (d, J=3.8 Hz, 1H), 7.35 (d, J=3.8 Hz, 1H), 4.47 (s, 2H); 13C NMR (125 MHz, D₂O) d 144.5, 136.8, 134.6, 131.5, 127.7, 126.1, 124.5, 123.5, 122.1, 30.8. HRMS calculated for C14H12N2O2 [M+H]+, 225.0248, found 225.0105, UPLC (254 nm)>99%.

### Compound AC-Boc: tert-butyl ((5-(4-(furan-2-yl)pyridin-3-yl)furan-2-yl)methyl)carbamate

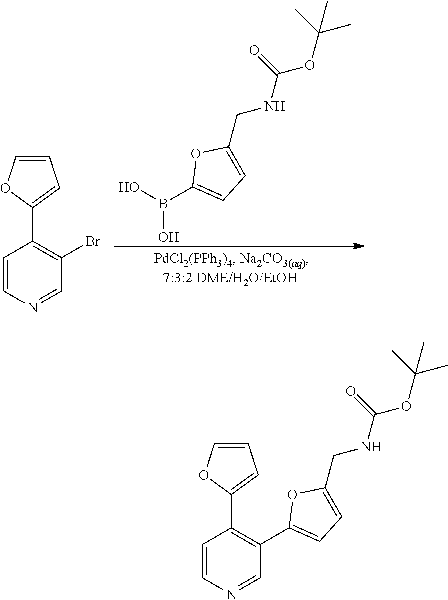 [SMILES: Brc1cnccc1-c1ccco1 ; CC(C)(C)OC(=O)NCc1ccc(B(O)O)o1 ; CC(C)(C)OC(=O)NCc1ccc(-c2cnccc2-c2ccco2)o1]

To a 5 mL microwave vial (Biotage) was added 3-bromo-4-(furan-2-yl)pyridine (83.1 mg, 0.371 mmol), (5-(((tert-butoxycarbonyl)amino)methyl)furan-2-yl)boronic acid (89.0 mg, 0.369 mmol) and bis(triphenylphosphine)palladium(II) dichloride (27.0 mg, 0.0385 mmol). The vial was purged with argon for 5 minutes followed by adding the degassed solvent of DME/H₂O/EtOH (7:3:2, v:v:v, 2.0 mL) and degassed 2 M Na₂CO₃ (0.7 mL). The vial was capped and placed in a Biotage Initiator+ microwave and heated to 140° C. for 5 minutes on normal absorption. The contents of the flask were cooled to rt, transferred to a separatory funnel, diluted with CH₂Cl₂ (25 mL), washed with water (20 mL), followed by saturated NaCl (20 mL), dried over Na₂SO₄, gravity filtered, the solvent was removed in vacuo and the residue was chromatographed on silica gel using a gradient elution (EtOAc/Hex, 10:90, v/v to EtOAc/Hex, 50:50, v/v, TLC: EtOAc/Hex, 50:50, v/v, Rf=0.36) to afford AC-Boc (53.0 mg, 43% yield) as a yellow semisolid: 1H NMR (500 MHz, CDCl₃) d 8.59 (s, 1H), 8.50 (d, J=5.4 Hz, 1H), 7.62 (d, J=6.1 Hz, 1H), 7.46 (d, J=1.25 Hz, 1H), 6.39 (m, 1H), 6.31 (m, 1H), 6.09 (d, J=3.1 Hz, 1H), 4.89 (bs, 1H), 4.25 (d, J=4.4 Hz, 2H), 1.38 (s, 9H).

### Compound AC: (5-(4-(furan-2-yl)pyridin-3-yl)furan-2-yl)methanamine Dihydrochloride

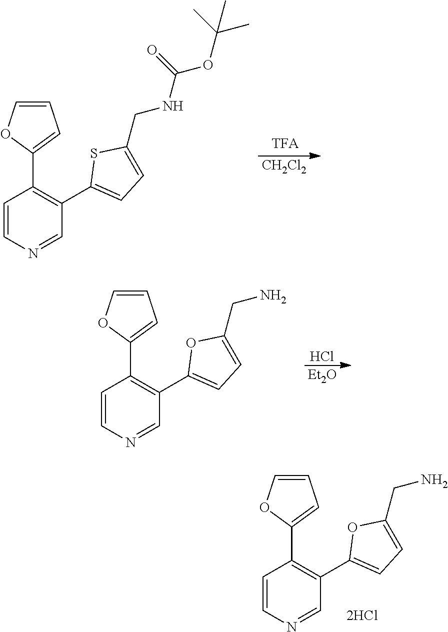 [SMILES: "Et2O" ; CC(C)(C)OC(=O)NCc1ccc(-c2cnccc2-c2ccco2)s1 ; "CH2Cl2" ; NCc1ccc(-c2cnccc2-c2ccco2)o1 ; Cl ; NCc1ccc(-c2cnccc2-c2ccco2)o1]

To a solution of AC-Boc (53.0 mg, 0.156 mmol) in dichloromethane (1 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford AC (31.0 mg, 63% yield) as a yellow solid: 1H NMR (500 MHz, D₂O) d 8.65 (s, 1H), 8.56 (d, J=5.8 Hz, 1H), 7.97 (d, J=5.8 Hz, 1H), 7.72 (m, 1H), 6.74 (d, J=3.4 Hz, 1H), 6.65 (d, J=3.4 Hz, 1H), 6.60 (m, 1H), 6.41 (d, J=3.6 Hz, 1H), 4.26 (s, 2H); 13C NMR (125 MHz, D₂O) d 144.4, 143.5, 142.6, 141.4, 141.2, 140.2, 139.2, 135.4, 118.1, 116.9, 110.2, 108.1, 107.2, 30.8. HRMS calculated for C14H13N2O2 [M+H]+, 241.0977, found 241.0979, UPLC (254 nm)>95%.

### Compound AD-Boc: tert-butyl ((5-(4-(furan-2-yl)pyridin-3-yl)thiophene-2-yl)methyl)carbamate

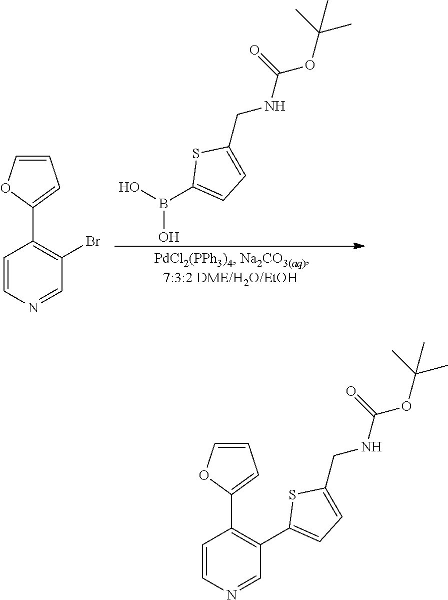 [SMILES: Brc1cnccc1-c1ccco1 ; CC(C)(C)OC(=O)NCc1ccc(B(O)O)s1 ; CC(C)(C)OC(=O)NCc1ccc(-c2cnccc2-c2ccco2)s1]

To a 5 mL microwave vial (Biotage) was added 3-bromo-4-(furan-2-yl)pyridine (76.0 mg, 0.339 mmol), (5-(((tert-butoxycarbonyl)amino)methyl)thiophen-2-yl)boronic acid (87.0 mg, 0.338 mmol) and bis(triphenylphosphine)palladium(II) dichloride (25.0 mg, 0.0356 mmol). The vial was purged with argon for 5 minutes followed by adding the degassed solvent of DME/H₂O/EtOH (7:3:2, v:v:v, 2.0 mL) and degassed 2 M Na₂CO₃ (0.75 mL). The vial was capped and placed in a Biotage Initiator+ microwave and heated to 140° C. for 5 minutes on normal absorption. The contents of the flask were cooled to rt, transferred to a separatory funnel, diluted with CH₂Cl₂ (25 mL), washed with water (20 mL), followed by saturated NaCl (20 mL), dried over Na₂SO₄, gravity filtered, the solvent was removed in vacuo and the residue was chromatographed on silica gel using a gradient elution (EtOAc/Hex, 15:85, v/v to EtOAc/Hex, 50:50, v/v, TLC: EtOAc/Hex, 50:50, v/v, Rf=0.44) to afford AD-Boc (59.0 mg, 50% yield) as a yellow semisolid: 1H NMR (500 MHz, CDCl₃) d 8.51 (d, J=5.5 Hz, 1H), 8.46 (s, 1H), 7.71 (d, J=5.5 Hz, 1H), 7.44 (m, 1H), 6.91 (d, J=2.8 Hz, 1H), 6.82 (d, J=3.5 Hz, 1H), 6.31 (dd, J=3.4 Hz, J=1.8 Hz, 1H), 6.03 (d, J=3.4 Hz, 1H), 5.01 (bs, 1H), 4.42 (m, 2H), 1.40 (s, 9H).

### Compound AD: (5-(4-(furan-2-yl)pyridin-3-yl)thiophene-2-yl)methanamine Dihydrochloride

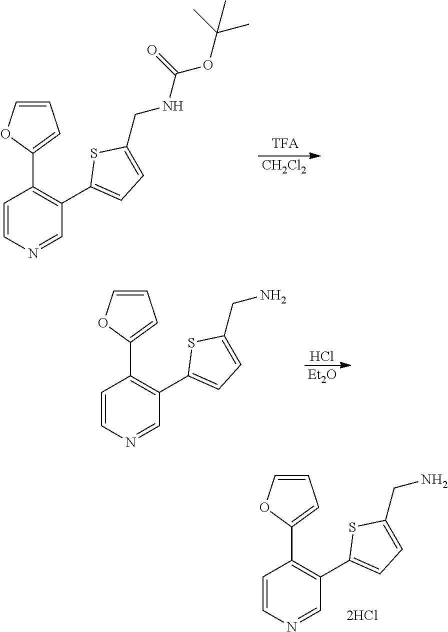 [SMILES: "Et2O" ; CC(C)(C)OC(=O)NCc1ccc(-c2cnccc2-c2ccco2)s1 ; "CH2Cl2" ; NCc1ccc(-c2cnccc2-c2ccco2)s1 ; Cl ; NCc1ccc(-c2cnccc2-c2ccco2)s1]

To a solution of AD-Boc (59.0 mg, 0.166 mmol) in dichloromethane (1 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford AD (40.0 mg, 73% yield) as a yellow solid: 1H NMR (500 MHz, D₂O) d 8.61 (d, J=6.5 Hz, 1H), 8.60 (s, 1H), 8.16 (d, J=6.5 Hz, 1H), 7.74 (d, J=1.6 Hz, 1H), 7.32 (d, J=3.6 Hz, 1H), 7.17 (d, J=3.6 Hz, 1H), 6.53 (dd, J=3.7 Hz, J=1.7 Hz, 1H), 6.32 (d, J=3.7 Hz, 1H), 4.46 (s, 2H); 13C NMR (125 MHz, D₂O) d 140.9, 140.3, 138.2, 136.9, 135.9, 130.6, 130.1, 123.7, 123.0, 120.4, 114.8, 111.1, 106.7, 30.8. HRMS calculated for C14H13N2OS [M+H]+, 257.0749, found 257.0780, UPLC (254 nm)=90%.

### Compound AE-Boc: tert-Butyl ((5-(4-(furan-3-yl)pyridin-3-yl)furan-2-yl)methyl)carbamate

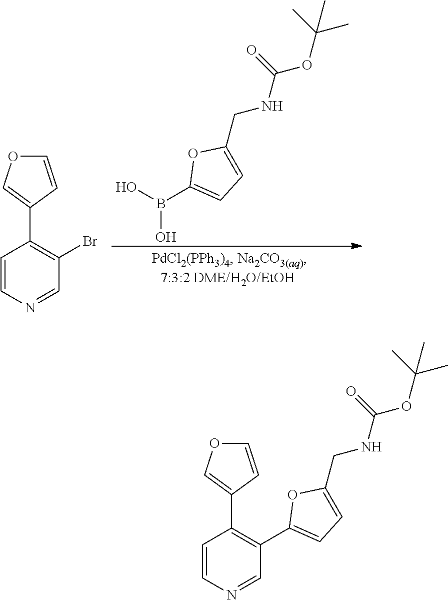 [SMILES: Brc1cnccc1-c1ccoc1 ; CC(C)(C)OC(=O)NCc1ccc(B(O)O)o1 ; CC(C)(C)OC(=O)NCc1ccc(-c2cnccc2-c2ccoc2)o1]

To a 5 mL microwave vial (Biotage) was added 3-bromo-4-(furan-3-yl)pyridine (75.0 mg, 0.335 mmol), (5-(((tert-butoxycarbonyl)amino)methyl)furan-2-yl)boronic acid (81.0 mg, 0.336 mmol) and bis(triphenylphosphine)palladium(II) dichloride (25.0 mg, 0.0356 mmol). The vial was purged with argon for 5 minutes followed by adding the degassed solvent of DME/H₂O/EtOH (7:3:2, v:v:v, 2.0 mL) and degassed 2 M Na₂CO₃ (0.7 mL). The vial was capped and placed in a Biotage Initiator+ microwave and heated to 140° C. for 5 minutes on normal absorption. The contents of the flask were cooled to rt, transferred to a separatory funnel, diluted with EtOAc (25 mL), washed with water (20 mL), followed by saturated NaCl (20 mL), dried over Na₂SO₄, gravity filtered, the solvent was removed in vacuo and the residue was chromatographed on silica gel using a gradient elution (EtOAc/Hex, 10:90, v/v to EtOAc/Hex, 50:50, v/v, TLC: EtOAc/Hex, 50:50, v/v, Rf=0.36) to afford AE-Boc (43.0 mg, 38% yield) as a semisolid: 1H NMR (500 MHz, CDCl₃) d 8.78 (s, 1H), 8.51 (d, J=5.1 Hz, 1H), 7.45-7.46 (m, 2H), 7.28 (d, J=5.1 Hz, 1H), 6.26-6.31 (m, 3H), 4.98 (bs, 1H), 4.30 (d, J=5.2 Hz, 2H), 1.46 (s, 9H).

### Compound AE: (5-(4-(furan-3-yl)pyridin-3-yl)furan-2-yl)methanamine Dihydrochloride

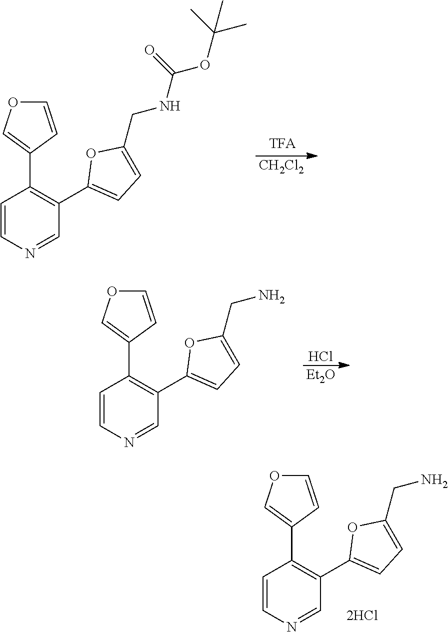 [SMILES: "Et2O" ; CC(C)(C)OC(=O)NCc1ccc(-c2cnccc2-c2ccoc2)o1 ; "CH2Cl2" ; NCc1ccc(-c2cnccc2-c2ccoc2)o1 ; Cl ; NCc1ccc(-c2cnccc2-c2ccoc2)o1]

To a solution of AE-Boc (43.0 mg, 0.131 mmol) in dichloromethane (0.75 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford AE (26.0 mg, 63% yield) as a yellow solid: 1H NMR (500 MHz, D₂O) d 8.86 (s, 1H), 8.62 (d, J=6.2 Hz, 1H), 8.01 (d, J=6.2 Hz, 1H), 7.84 (m, 1H), 7.63 (dd, J=1.7 Hz, 1H), 6.74 (d, J=3.5 Hz, 1H), 6.71 (d, J=3.5 Hz, 1H), 6.47 (m, 1H), 4.25 (s, 2H); 13C NMR (125 MHz, D₂O) d 143.3, 143.1, 143.0, 139.9, 139.4, 136.9, 135.8, 122.2, 121.8, 117.0, 108.9, 108.3, 104.7, 30.76. HRMS calculated for C14H13N2O2 [M+H]+, 241.0977, found 241.0988, UPLC (254 nm)=90%.

### Compound AF-Boc: tert-Butyl [5-(4-furan-3-yl-pyridin-3-yl)thiophene-2-ylmethyl]carbamate

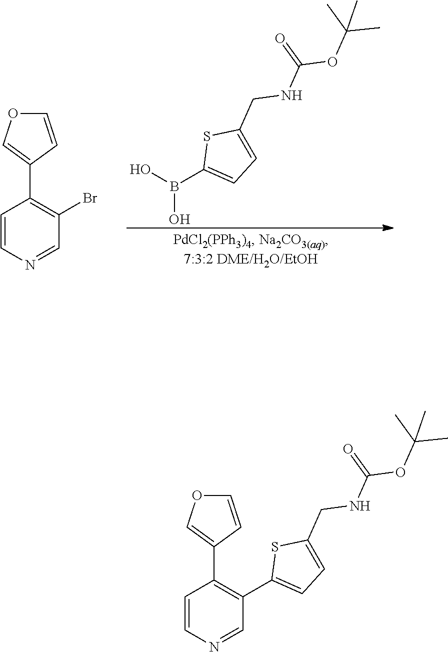 [SMILES: Brc1cnccc1-c1ccoc1 ; CC(C)(C)OC(=O)NCc1ccc(B(O)O)s1 ; CC(C)(C)OC(=O)NCc1ccc(-c2cnccc2-c2ccoc2)s1]

To a 5 mL microwave vial (Biotage) was added 3-bromo-4-(furan-3-yl)pyridine (80.0 mg, 0.357 mmol), (5-(((tert-butoxycarbonyl)amino)methyl)thiophen-2-yl)boronic acid (91.0 mg, 0.354 mmol) and bis(triphenylphosphine)palladium(II) dichloride (26.0 mg, 0.037 mmol). The vial was purged with argon for 5 minutes followed by adding the degassed solvent of DME/H₂O/EtOH (7:3:2, v:v:v, 2.0 mL) and degassed 2 M Na₂CO₃ (0.75 mL). The vial was capped and the stirring slurry was heated at 140° C. by microwave irradiation on normal absorption level for 5 minutes, cooled to rt, diluted with dichloromethane (25 mL), washed with water (20 mL) followed by saturated NaCl (20 mL), dried over Na₂SO₄, gravity filtered and the solvent was removed in vacuo to afford the crude material which was purified by flash chromatography using a gradient elution (EtOAc/Hex, 10:90, v/v to EtOAc/Hex, 50:50, v/v, TLC: 50% EtOAc/hexane, Rf=0.34) to afford the product AF-Boc (70.0 mg, 55% yield) as an off-white solid: 1H NMR (500 MHz, CDCl₃) d 8.58 (s, 1H), 8.53 (d, J=5.1 Hz, 1H), 7.38 (dd, J=1.7 Hz, 1H), 7.35 (m, 1H), 7.32 (d, J=5.1 Hz, 1H), 6.90 (d, J=3.4 Hz, 1H), 6.82 (d, J=3.4 Hz, 1H), 6.32 (m, 1H), 5.19 (bs, 1H), 4.48 (d, J=4.9 Hz, 2H), 1.46 (s, 9H).

### Compound AF: (5-(4-(furan-3-yl)pyridin-3-yl)thiophene-2-yl)methanamine Dihydrochloride

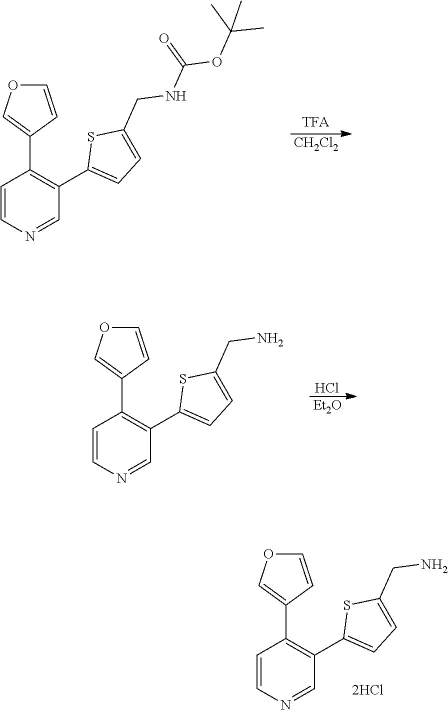 [SMILES: "Et2O" ; CC(C)(C)OC(=O)NCc1ccc(-c2cnccc2-c2ccoc2)s1 ; "CH2Cl2" ; NCc1ccc(-c2cnccc2-c2ccoc2)s1 ; Cl ; NCc1ccc(-c2cnccc2-c2ccoc2)s1]

To a solution of AF-Boc (70.0 mg, 0.196 mmol) in dichloromethane (1 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford AF (41.0 mg, 64% yield) as a yellow solid: 1H NMR (500 MHz, D₂O) d 8.62 (s, 1H), 8.56 (d, J=5.9 Hz, 1H), 7.82 (d, J=5.9 Hz, 1H), 7.57 (m, 1H), 7.52 (m, 1H), 7.25 (d, J=3.7 Hz, 1H), 7.12 (d, J=3.7 Hz, 1H), 6.45, (m, 1H), 4.41 (s, 2H); 13C NMR (125 MHz, D₂O) d 139.6, 139.2, 137.6, 137.3 (2 C), 131.5, 129.9, 123.5, 123.0 (2 C), 118.2, 115.2, 103.2, 30.8. HRMS calculated for C14H13N2OS [M+H]+, 257.0749, found 257.0757, UPLC (254 nm)=95%.

### Compound AG-Boc: tert-Butyl ((5-([3,4′-bipyridin]-3′-yl)furan-2-yl)methyl)carbamate

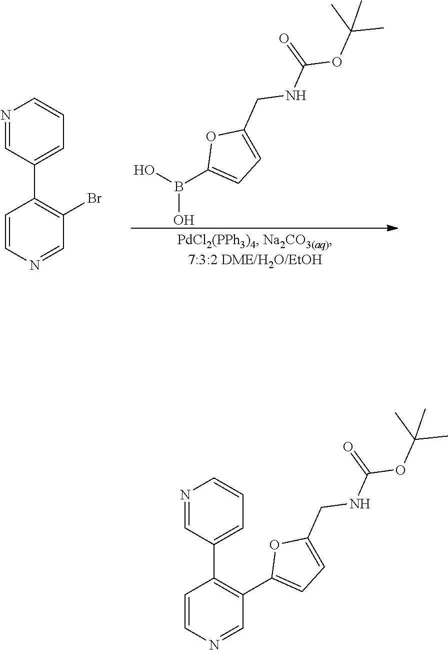 [SMILES: Brc1cnccc1-c1cccnc1 ; CC(C)(C)OC(=O)NCc1ccc(B(O)O)o1 ; CC(C)(C)OC(=O)NCc1ccc(-c2cnccc2-c2cccnc2)o1]

To a 5 mL microwave vial (Biotage) was added 3′-bromo-3,4′-bipyridine (104.0 mg, 0.442 mmol), (5-(((tert-butoxycarbonyl)amino)methyl)furan-2-yl)boronic acid (106.0 mg, 0.440 mmol) and bis(triphenylphosphine)palladium(II) dichloride (31.0 mg, 0.0442 mmol). The vial was purged with argon for 5 minutes followed by adding the degassed solvent of DME/H₂O/EtOH (7:3:2, v:v:v, 2.0 mL) and degassed 2 M Na₂CO₃ (0.75 mL). The vial was capped and the stirring slurry was heated at 140° C. by microwave irradiation on normal absorption level for 5 minutes, cooled to rt, diluted with EtOAc (30 mL), washed with water (15 mL) followed by saturated NaCl (15 mL), dried over Na₂SO₄, gravity filtered and the solvent was removed in vacuo to afford the crude material which was purified by flash chromatography using a gradient elution (EtOAc/Hex, 50:50, v/v to 100%, TLC: 100% EtOAc, Rf=0.22) to afford the product AG-Boc (57.0 mg, 37% yield) as a yellow solid: 1H NMR (500 MHz, CDCl₃) d 8.87 (s, 1H), 8.59 (d, J=4.1 Hz, 1H), 8.50 (d, J=5.0 Hz, 1H), 8.47 (m, 1H), 7.53 (ddd, J=7.8 Hz, J=1.9 Hz, 1H), 7.29 (dd, J=7.8 Hz, J=5.0 Hz, 1H), 7.15 (d, J=5.0 Hz, 1H), 6.08 (m, 1H), 5.87 (d, J=3.1 Hz, 1H), 4.81 (bs, 1H), 4.12 (d, J=5.3 Hz, 2H), 1.38 (s, 9H).

### Compound AG: (5-([3,4′-Bipyridin]-3′-yl)furan-2-yl)methanamine Trihydrochloride

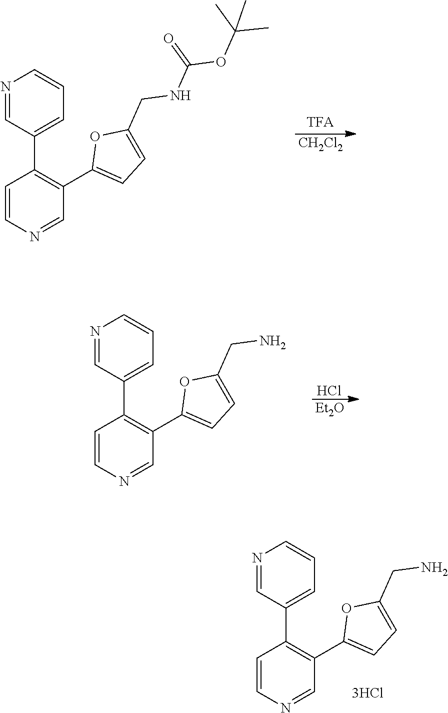 [SMILES: "Et2O" ; CC(C)(C)OC(=O)NCc1ccc(-c2cnccc2-c2cccnc2)o1 ; "CH2Cl2" ; NCc1ccc(-c2cnccc2-c2cccnc2)o1 ; Cl ; NCc1ccc(-c2cnccc2-c2cccnc2)o1]

To a solution of AF-Boc (57.0 mg, 0.162 mmol) in dichloromethane (0.75 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford AG (36.0 mg, 62% yield) as a yellow solid: 1H NMR (500 MHz, D₂O) d 9.04 (s, 1H), 8.80 (dd, J=5.5 Hz, J=1.4 Hz, 1H), 8.78 (d, J=1.8 Hz, 1H), 8.69 (d, J=5.5 Hz, 1H), 8.35 (ddd, J=8.1 Hz, J=1.8 Hz, 1H), 7.92 (ddd, J=8.1 Hz, J=5.6 Hz, J=0.5 Hz, 1H), 7.75 (d, J=5.6 Hz, 1H), 6.56 (d, J=3.5 Hz, 1H), 6.23 (d, J=3.5 Hz, 1H), 4.16 (s, 2H); 13C NMR (125 MHz, D₂O) d 143.8, 143.3, 140.1 (2 C), 140.0 (2 C), 138.9, 138.4, 131.2, 122.2, 121.9, 121.5, 108.9, 108.3, 30.8. HRMS calculated for C15H14N3O [M+H]+, 252.1137, found 252.1160, UPLC (254 nm)>99%.

### Compound AH-Boc: tert-Butyl ((5-([3,4′-bipyridin]-3′-yl)thiophene-2-yl)methyl)carbamate

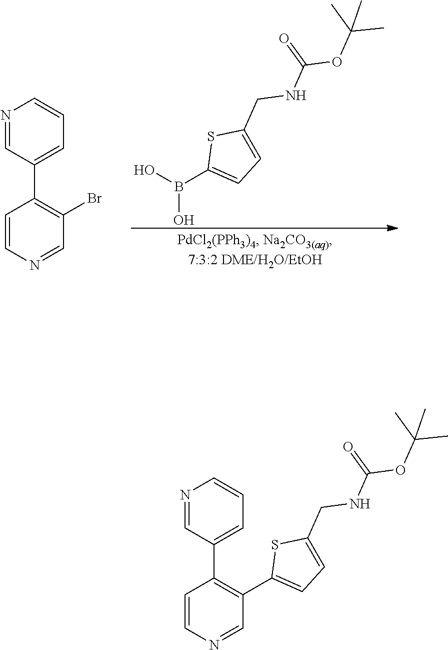 [SMILES: Brc1cnccc1-c1cccnc1 ; CC(C)(C)OC(=O)NCc1ccc(B(O)O)s1 ; CC(C)(C)OC(=O)NCc1ccc(-c2cnccc2-c2cccnc2)s1]

To a 5 mL microwave vial (Biotage) was added 3′-bromo-3, 4′-bipyridine (77.0 mg, 0.328 mmol), (5-(((tert-butoxycarbonyl)amino)methyl)thiophen-2-yl)boronic acid (84.0 mg, 0.327 mmol) and bis(triphenylphosphine)palladium(II) dichloride (22.0 mg, 0.0313 mmol). The vial was purged with argon for 5 minutes followed by adding the degassed solvent of DME/H₂O/EtOH (7:3:2, v:v:v, 2.0 mL) and degassed 2 M Na₂CO₃ (0.75 mL). The vial was capped and the stirring slurry was heated at 140° C. by microwave irradiation on normal absorption level for 5 minutes, cooled to rt, diluted with EtOAc (30 mL), washed with water (15 mL) followed by saturated NaCl (15 mL), dried over Na₂SO₄, gravity filtered and the solvent was removed in vacuo to afford the crude material which was purified by flash chromatography using a gradient elution (EtOAc/Hex, 50:50, v/v to 100%, TLC: 100% EtOAc, Rf=0.22) to afford the product AH-Boc (49.0 mg, 41% yield) as a yellow semisolid: 1H NMR (500 Hz, CDCl₃) d 8.56-8.80 (m, 4H), 7.63 (d, J=7.8 Hz, 1H), 7.32-7.35 (m, 2H), 6.80 (d, J=3.3 Hz, 1H), 6.65 (d, J=3.3 Hz, 1H), 4.94 (bs, 1H), 4.40 (d, J=5.0 Hz, 2H), 1.45 (s, 9H).

### Compound AH: (5-([3,4′-Bipyridin]-3′-yl)thiophene-2-yl)methanamine Trihydrochloride

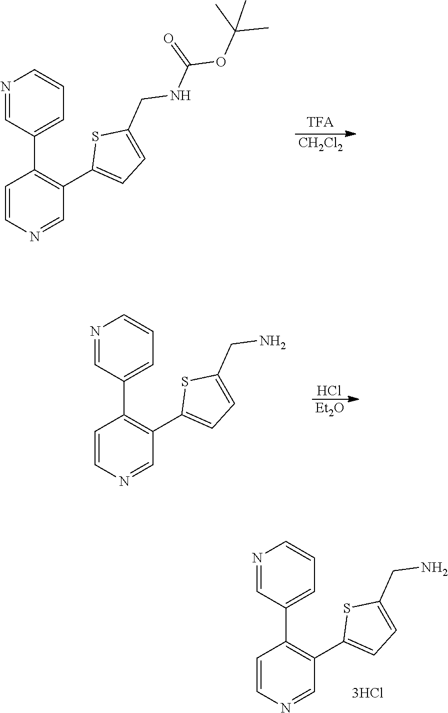 [SMILES: "Et2O" ; CC(C)(C)OC(=O)NCc1ccc(-c2cnccc2-c2cccnc2)s1 ; "CH2Cl2" ; NCc1ccc(-c2cnccc2-c2cccnc2)s1 ; Cl ; NCc1ccc(-c2cnccc2-c2cccnc2)s1]

To a solution of AH-Boc (49.0 mg, 0.133 mmol) in dichloromethane (0.75 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford AH (31.0 mg, 65% yield) as a white solid: 1H NMR (500 MHz, D₂O) d 9.00 (s, 1H), 8.85 (d, J=5.8 Hz, 1H), 8.81-8.84 (m, 2H), 8.48 (ddd, J=8.1 Hz, J=1.6 Hz, 1H), 8.03 (d, J=5.8 Hz, 1H), 8.00 (dd, J=8.1 Hz, J=5.8 Hz, 1H), 7.18 (d, J=3.7 Hz, 1H), 7.06 (d, J=3.7 Hz, 1H), 4.34 (s, 2H); 13C NMR (125 MHz, D₂O) d 141.9, 139.1, 138.5, 137.1, 136.9, 136.2, 131.6, 129.4, 129.0, 125.5, 124.7, 124.0, 121.0, 120.5, 30.8. HRMS calculated for C15H14N3S [M+H]+, 268.0908, found 268.0921, UPLC (254 nm)>99%.

### Compound AI-Boc: tert-Butyl ((5-([4,4′-bipyridin]-3′-yl)furan-2-yl)methyl)carbamate

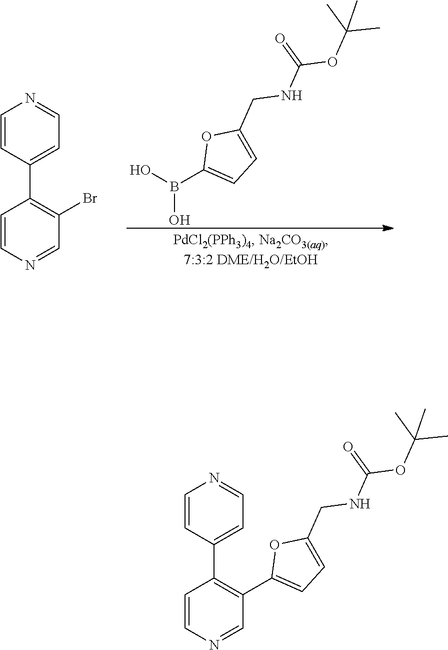 [SMILES: Brc1cnccc1-c1ccncc1 ; CC(C)(C)OC(=O)NCc1ccc(B(O)O)o1 ; CC(C)(C)OC(=O)NCc1ccc(-c2cnccc2-c2ccncc2)o1]

To a 5 mL microwave vial (Biotage) was added 3′-bromo-4,4′-bipyridine (63.0 mg, 0.268 mmol), (5-(((tert-butoxycarbonyl)amino)methyl)furan-2-yl)boronic acid (65.0 mg, 0.270 mmol) and bis(triphenylphosphine)palladium(II) dichloride (24.0 mg, 0.0342 mmol). The vial was purged with argon for 5 minutes followed by adding the degassed solvent of DME/H₂O/EtOH (7:3:2, v:v:v, 2.0 mL) and degassed 2 M Na₂CO₃ (0.75 mL). The vial was capped and the stirring slurry was heated at 140° C. by microwave irradiation on normal absorption level for 5 minutes, cooled to rt, diluted with EtOAc (30 mL), washed with water (15 mL) followed by saturated NaCl (15 mL), dried over Na₂SO₄, gravity filtered and the solvent was removed in vacuo to afford the crude material which was purified by flash chromatography using a gradient elution (EtOAc/Hex, 10:90, v/v to 100%, TLC: 25% EtOAc/Hex, v/v, Rf=0.19) to afford the product AI-Boc (28.0 mg, 30% yield) as a yellow semisolid: 1H NMR (500 MHz, CDCl₃) d 8.97 (s, 1H), 8.68 (d, J=6.0 Hz, 2H), 8.59 (d, J=5.0 Hz, 1H), 7.23 (d, J=6.0 Hz, 2H), 7.19 (d, J=5.0 Hz, 1H), 6.16 (m, 1H), 5.95 (d, J=2.8 Hz, 1H), 4.81 (bs, 1H), 4.21 (d, J=5.2 Hz, 2H), 1.46 (s, 9H).

### Compound AI: (5-([4,4′-Bipyridin]-3′-yl)furan-2-yl)methanamine Trihydrochloride

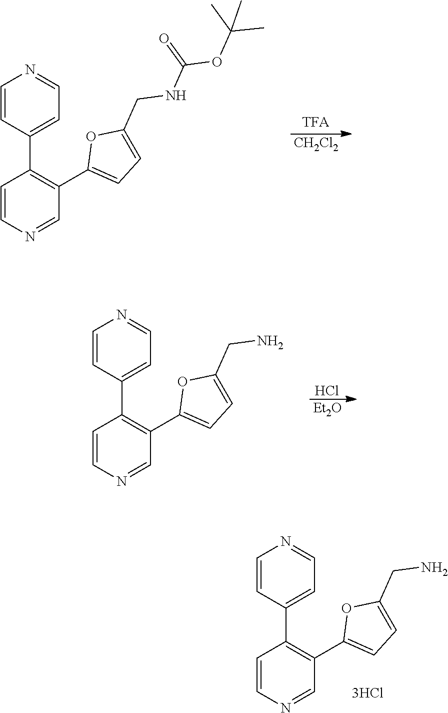 [SMILES: "Et2O" ; CC(C)(C)OC(=O)NCc1ccc(-c2cnccc2-c2ccncc2)o1 ; "CH2Cl2" ; NCc1ccc(-c2cnccc2-c2ccncc2)o1 ; Cl ; NCc1ccc(-c2cnccc2-c2ccncc2)o1]

To a solution of AI-Boc (28.0 mg, 0.0797 mmol) in dichloromethane (0.75 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford AI (18.0 mg, 63% yield) as a yellow solid: 1H NMR (500 MHz, D₂O) d 9.23 (s, 1H), 8.96 (d, J=6.6 Hz, 2H), 8.85 (d, J=5.8 Hz, 1H), 8.20 (d, J=6.6 Hz, 2H), 8.05 (d, J=5.8 Hz, 1H), 6.61 (d, J=3.3 Hz, 1H), 6.41 (d, J=3.3 Hz, 1H), 4.18 (s, 2H); 13C NMR (125 MHz, D₂O) d 149.8, 144.7, 142.9, 141.8, 137.8 (2 C), 137.2, 136.6, 123.2, 123.0, 122.5 (2 C), 110.8, 108.8, 30.8. HRMS calculated for C15H14N3O [M+H]+, 252.1137, found 252.1141, UPLC (254 nm)>95%.

### Compound AJ-Boc: tert-Butyl ((5-([4,4′-bipyridin]-3′-yl)thiophene-2-yl)methyl)carbamate

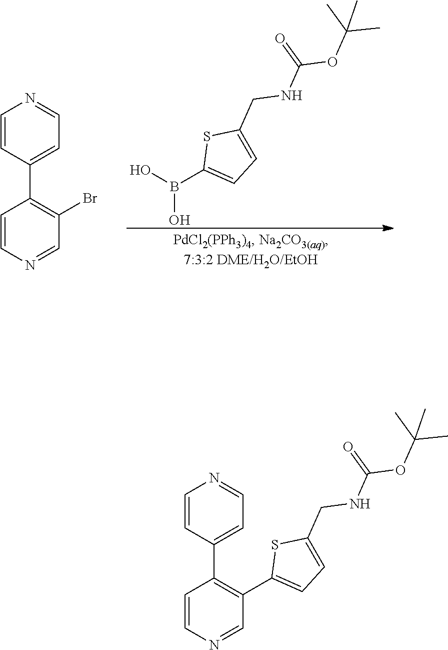 [SMILES: Brc1cnccc1-c1ccncc1 ; CC(C)(C)OC(=O)NCc1ccc(B(O)O)s1 ; CC(C)(C)OC(=O)NCc1ccc(-c2cnccc2-c2ccncc2)s1]

To a 5 mL microwave vial (Biotage) was added 3′-bromo-4, 4′-bipyridine (72.0 mg, 0.306 mmol), (5-(((tert-butoxycarbonyl)amino)methyl)thiophen-2-yl)boronic acid (78.0 mg, 0.303 mmol) and bis(triphenylphosphine)palladium(II) dichloride (22.0 mg, 0.0313 mmol). The vial was purged with argon for 5 minutes followed by adding the degassed solvent of DME/H₂O/EtOH (7:3:2, v:v:v, 2.0 mL) and degassed 2 M Na₂CO₃ (0.75 mL). The vial was capped and the stirring slurry was heated at 140° C. by microwave irradiation on normal absorption level for 5 minutes, cooled to rt, diluted with EtOAc (30 mL), washed with water (15 mL) followed by saturated NaCl (15 mL), dried over Na₂SO₄, gravity filtered and the solvent was removed in vacuo to afford the crude material which was purified by flash chromatography using a gradient elution (EtOAc/Hex, 50:50, v/v to 100% EtOAc, TLC: 100% EtOAc, Rf=0.20) to afford the product AJ-Boc (43.0 mg, 39% yield) as a yellow semisolid: 1H NMR (500 MHz, CDCl₃) d 8.76 (s, 1H), 8.60-8.64 (m, 3H), 7.25 (d, J=5.0 Hz, 1H), 7.20 (d, J=5.8 Hz, 2H), 6.79 (d, J=3.4 Hz, 1H), 6.60 (d, J=3.4 Hz, 1H), 5.12 (bs, 1H), 4.41 (d, J=5.1 Hz, 2H), 1.45 (s, 9H).

### Compound AJ: (5-([4,4′-Bipyridin]-3′-yl)thiophene-2-yl)methanamine Trihydrochloride

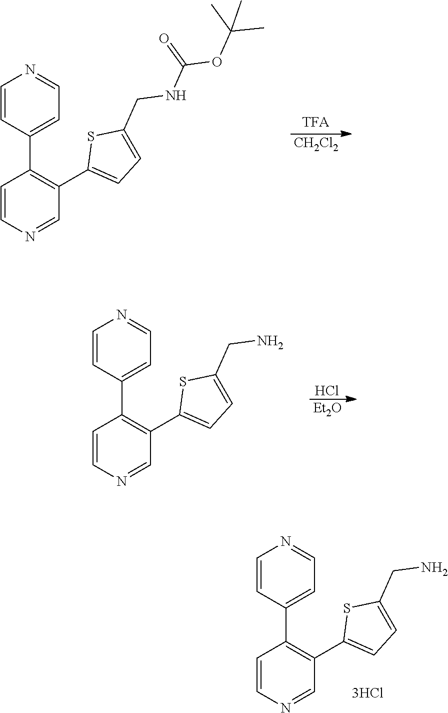 [SMILES: "Et2O" ; CC(C)(C)OC(=O)NCc1ccc(-c2cnccc2-c2ccncc2)s1 ; "CH2Cl2" ; NCc1ccc(-c2cnccc2-c2ccncc2)s1 ; Cl ; NCc1ccc(-c2cnccc2-c2ccncc2)s1]

To a solution of AJ-Boc (43.0 mg, 0.117 mmol) in dichloromethane (0.75 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford AJ (31.0 mg, 70% yield) as a yellow solid. 1H NMR (500 MHz, D₂O) d 9.01 (s, 1H), 8.87 (d, J=5.7 Hz, 1H), 8.82 (d, J=6.8 Hz, 2H), 8.08 (d, J=6.8 Hz, 2H), 8.00 (d, J=5.7 Hz, 1H), 7.17 (d, J=3.8 Hz, 1H), 7.00 (d, J=3.8 Hz, 1H), 4.35 (s, 2H); 13C NMR (125 MHz, D₂O) d 147.9, 142.3, 139.2, 137.5, 135.7 (2 C), 131.6, 129.2, 124.8, 124.7, 124.0, 121.1 (2 C), 120.2, 30.8. HRMS calculated for C15H14N3S [M+H]+, 268.0908, found 268.0907, UPLC (254 nm)>95%.

### Compound AK-Boc: tert-Butyl ((5-(4-(pyrimidin-5-yl)pyridine-3-yl)furan-2-yl)methyl)carbamate

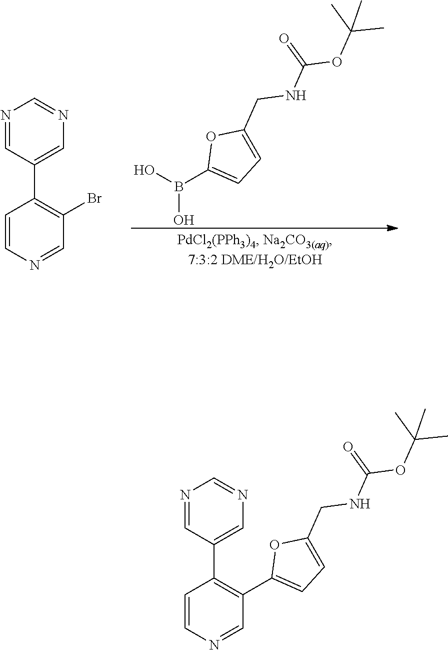 [SMILES: Brc1cnccc1-c1cncnc1 ; CC(C)(C)OC(=O)NCc1ccc(B(O)O)o1 ; CC(C)(C)OC(=O)NCc1ccc(-c2cnccc2-c2cncnc2)o1]

To a 5 mL microwave vial (Biotage) was added 5-(3-bromopyridin-4-yl)pyrimidine (102. mg, 0.430 mmol), (5-(((tert-butoxycarbonyl)aminuteso)methyl)furan-2-yl)boronic acid (104. mg, 0.431 mmol) and bis(triphenylphosphine)palladium(II) dichloride (29.0 mg, 0.0413 mmol). The vial was purged with argon for 5 minutes followed by adding the degassed solvent of DME/H₂O/EtOH (7:3:2, v:v:v, 2.0 mL) and degassed 2 M Na₂CO₃ (0.75 mL). The vial was capped and the stirring slurry was heated at 140° C. by microwave irradiation on normal absorption level for 5 minutes, cooled to rt, diluted with EtOAc (30 mL), washed with water (15 mL) followed by saturated NaCl (15 mL), dried over Na₂SO₄, gravity filtered and the solvent was removed in vacuo to afford the crude material which was purified by flash chromatography using a gradient elution (EtOAc/Hex, 30:70, v/v to 100% EtOAc, TLC: 100% EtOAc, Rf=0.28) to afford the product AK-Boc (79.0 mg, 52% yield) as a yellow semisolid: 1H NMR (500 MHz, CDCl₃) d 9.18 (s, 1H), 8.87 (s, 1H), 8.60 (s, 2H), 8.55 (d, J=5.0 Hz, 1H), 7.17 (d, J=5.0 Hz, 1H), 6.15 (d, J=2.3 Hz, 1H), 6.06 (d, J=2.3 Hz, 1H), 4.91 (bs, 1H), 4.11 (d, J=5.4 Hz, 2H), 1.37 (s, 9H).

### Compound AK: (5-(4-(Pyrimidin-5-yl)pyridin-3-yl)furan-2-yl)methanamine Trihydrochloride

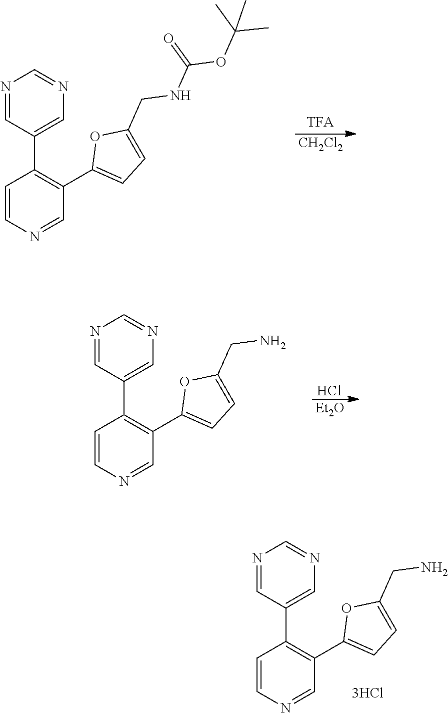 [SMILES: "Et2O" ; CC(C)(C)OC(=O)NCc1ccc(-c2cnccc2-c2cncnc2)o1 ; "CH2Cl2" ; NCc1ccc(-c2cnccc2-c2cncnc2)o1 ; Cl ; NCc1ccc(-c2cnccc2-c2cncnc2)o1]

To a solution of AK-Boc (79.0 mg, 0.224 mmol) in dichloromethane (0.75 mL), cooled to 0° C. in an external ice water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford AK (37.0 mg, 46% yield) as a light yellow solid: 1H NMR (500 MHz, D₂O) d 9.29 (s, 1H), 9.14 (s, 1H), 8.92 (s, 2H), 8.78 (d, J=5.8 Hz, 1H), 8.00 (d, J=5.8 Hz, 1H), 6.61 (d, J=3.2 Hz, 1H), 6.39 (d, J=3.2 Hz, 1H), 4.18 (s, 2H); 13C NMR (125 MHz, D₂O) d 153.4, 151.7 (2 C), 144.0, 142.8, 141.5, 137.4, 136.9, 126.8, 123.5, 123.4, 109.9, 108.6, 30.8. HRMS calculated for C14H13N4O [M+H]+, 253.1089, found 253.1098, UPLC (254 nm)>95%.

### Compound AL-Boc: tert-Butyl ((5-(4-(pyrimidin-5-yl)pyridin-3-yl)thiophene-2-yl)methyl)carbamate

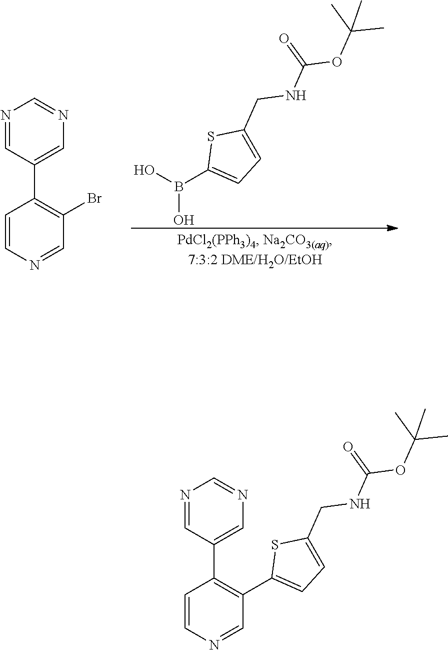 [SMILES: Brc1cnccc1-c1cncnc1 ; CC(C)(C)OC(=O)NCc1ccc(B(O)O)s1 ; CC(C)(C)OC(=O)NCc1ccc(-c2cnccc2-c2cncnc2)s1]

To a 5 mL microwave vial (Biotage) was added 5-(3-bromopyridin-4-yl)pyrimidine (108. mg, 0.458 mmol), (5-(((tert-butoxycarbonyl)amino)methyl)thiophen-2-yl)boronic acid (121. mg, 0.471 mmol) and bis(triphenylphosphine)palladium(II) dichloride (29.0 mg, 0.0413 mmol). The vial was purged with argon for 5 minutes followed by adding the degassed solvent of DME/H₂O/EtOH (7:3:2, v:v:v, 2.0 mL) and degassed 2 M Na2CO3 (0.75 mL). The vial was capped and the stirring slurry was heated at 140° C. by microwave irradiation on normal absorption level for 5 minutes, cooled to rt, diluted with EtOAc (30 mL), washed with water (15 mL) followed by saturated NaCl (15 mL), dried over Na₂SO₄, gravity filtered and the solvent was removed in vacuo to afford the crude material which was purified by flash chromatography using a gradient elution (EtOAc/Hex, 30:70, v/v to 100% EtOAc, TLC: 100% EtOAc, Rf=0.29) to afford the product AL-Boc (84.0 mg, 50% yield) as a light yellow semisolid: 1H NMR (500 MHz, CDCl₃) d 9.21 (s, 1H), 8.79 (s, 1H), 8.68 (d, J=5.0 Hz, 2H), 8.66 (s, 2H), 7.30 (d, J=5.0 Hz, 1H), 6.83 (m, 1H), 6.68 (d, J=3.4 Hz, 1H), 5.02 (bs, 1H), 4.41 (d, J=5.1 Hz, 2H), 1.45 (s, 9H).

### Compound AL: (5-(4-(Pyrimidin-5-yl)pyridin-3-yl)thiophene-2-yl)methanamine Trihydrochloride

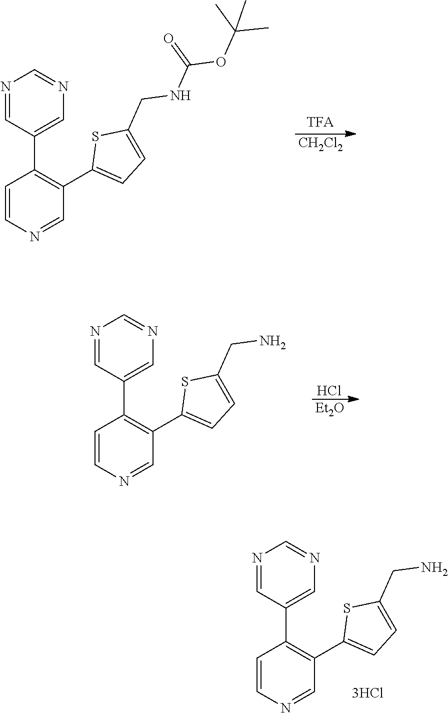 [SMILES: "Et2O" ; CC(C)(C)OC(=O)NCc1ccc(-c2cnccc2-c2cncnc2)s1 ; "CH2Cl2" ; NCc1ccc(-c2cnccc2-c2cncnc2)s1 ; Cl ; NCc1ccc(-c2cnccc2-c2cncnc2)s1]

To a solution of AL-BOC (84.0 mg, 0.228 mmol) in dichloromethane (0.75 mL), cooled to 0° C. in an external ice-water bath, was added trifluoroacetic acid (0.5 mL). The ice bath was removed and the resultant solution was stirred at ambient temperature for 2 hours. The reaction mixture was subsequently chilled in an external ice-water bath followed by dropwise adding saturated sodium carbonate (5 mL). The resultant mixture was stirred at room temperature for one hour, diluted with water (20 mL) and extracted with dichloromethane (2×25 mL). The organic solution was dried over Na₂SO₄, gravity filtered and the solvent was removed in vacuo to afford the crude material which was treated with HCl in ether (1 mL, dropwise). The solid material was collected and the residual solvent was removed in vacuo to afford AL (68.0 mg, 79% yield) as a yellow solid. 1H NMR (500 MHz, D₂O) d 9.21 (s, 1H), 9.02 (s, 1H), 8.87 (d, J=6.0 Hz, 1H), 8.84 (s, 2H), 8.13 (d, J=6.0 Hz, 1H), 7.20 (d, J=3.8 Hz, 1H), 7.12 (d, J=3.8 Hz, 1H), 4.35 (s, 2H); 13C NMR (125 MHz, D₂O) d 151.5, 150.4 (2 C), 143.0, 136.9, 135.3, 131.8, 129.1, 126.2, 124.8, 124.4, 124.0, 121.4, 30.8. HRMS calculated for C14H13N4S [M+H]+, 269.0861, found 269.0868, UPLC (254 nm)>95%.

### Example 2: Methods for Blocking CYP2A6 and/or UGT2B10 Mediated Nicotine Metabolism

CYP2A6 Inhibition Assays.

The inhibition activities of the nicotine analogues (NA) against CYP2A6 was determined using either 1 μM or 10 μM NA as an initial screen. Incubations were performed in pooled human liver microsomes (mixed gender, pool of 50 donors; obtained from Xenotech LLC, Lenexa, Kans.) in incubations using coumarin (Sigma-Aldrich, USA) as the CYP2A6 probe substrate. Coumarin was prepared as 20 mM stock solutions in DMSO and stored in aliquots at −80° C. Inhibition assays for each NA against CYP2A6 were performed after pre-incubation of a reaction mixture containing pooled human liver microsomes (0.1 mg/mL), the tested NA (1 or 10 μM) in 0.1% DMSO, coumarin (2.5 uM; approximating the known K_{M} for CYP2A6 against coumarin), 100 mM potassium phosphate buffer (pH 7.4), and magnesium chloride (3 mM) in a final reaction volume of 50 μL for 5 minutes in a 37° C. water bath. The reaction was initiated by the addition of an NADPH-regenerating system (1.3 mM NADP, 3.3 mM glucose 6-phosphate and 0.4 U/mL glucose 6-phosphate dehydrogenase; Corning; Bedford, Mass.) and incubated for 15 minutes at 37° C. Reactions were terminated by the addition of 50 μL of stop solution (acetonitrile/methanol; 1:1, v/v). Samples were mixed on a vortex mixer and centrifuged at 13,500×g for 15 minutes at 4° C. The supernatant (˜75 μL) was then transferred to a clean tube, and the metabolite (7-hydroxycoumarin) was detected using an ultra-pressure liquid chromatograph (UPLC; Waters Acquity; Waters Corp, Milford, Mass.) coupled to an electron triple-quadrupole mass spectrometer (Waters Xevo TDQ; Waters Corp, Milford, Mass.) by multiple reaction monitoring (MRM) analysis as described below.

As a positive control for every CYP2A6 inhibition experiment, 1 μM methoxsalen was added instead of the test agent. As a negative control experiment, only vehicle (0.1% DMSO) was added (no inhibitor or test agent added).

Inhibition assays for CYPs 1A2, 2B6, 2C8, 2C9, 2C19, 2D6, 2E1 and 3A4 were performed as described above for CYP2A6 using phenacetin, bupropion, amodiaquine, diclofenac, omeprazole, dextromethorphan, chlorzoxazone and midazolam (all purchased from Sigma-Aldrich; St. Louis, Mo.), respectively, as the corresponding probe substrates; with concentrations indicated in Table 1. Positive controls for the inhibition of each enzyme included: furafylline (1 μM), clopidogrel (1 μM), montelukast (5 μM), sulfaphenazole (1 μM), tranylcypromine (10 μM), quinidine (1 μM), chlomethiazole (10 μM) and ketoconazole (1 μM) for CYPs 1A2, 2B6, 2C8, 2C9, 2C19, 2D6, 2E1 and 3A4, respectively (see Table 1).

TABLE 1
Probe substrates, concentrations and reference inhibitors used in CYP450 assays.

| | | Substrate | | Positive |
|---|---|---|---|---|
| Enzyme | Probe Substrate | Concentration (μM) | Metabolite | Control Inhibitor |
| CYP1A2 | Phenacetin | 10 | Acetaminophen | Furafylline |
| CYP2A6 | Coumarin | 2.5 | 7-hydroxycoumarin | Tranylcypromine |
| CYP2B6 | Bupropion | 100 | Hydroxybupropion | Clopidogrel |
| CYP2C8 | Amodiaquine | 2.0 | Desethylaminodiaquine | Montelukast |
| CYP2C9 | Diclofenac | 10 | 4′-hydroxydiclofenac | Sulfaphenazole |
| CYP2C19 | Omeprazole | 1.0 | 5-hydroxyomeprazole | Tranylcypromine |
| CYP2D6 | Dextromethorphan | 5.0 | Dextrophan | Quinidine |
| CYP2E1 | Chlorzoxazone | 100 | 6-hydroxychlorzoxazone | Chlomethiazole |
| CYP3A4 | Midazolam | 5.0 | 1-hydroxymidazolam | Ketoconazole |

The activity of CYPs 3A4 (midazolam), 2D6 (dextromethorphan), 2C9 (diclofenac), 2C19 (omeprazole) and 1A2 (phenacetin) was determined using a cocktail method containing five probe substrates in a single reaction in incubation mixtures performed essentially as described above for CYP2A6, with agents, reference inhibitor, or vehicle control incubated with a cocktail containing the five probe substrates (indicated above) at a concentration similar to their known, respective K_{M} values as indicated in Table 1. The metabolites of CYPs 3A4, 2D6, 2C9, 2C19 and 1A2, [hydroxyl (OH)-midazolam, dextrophan, OH-diclofenac, OH-omeprazole and acetaminophen, respectively] were detected simultaneously on five different channels using Waters Acquity UPLC coupled to a mass spectrometer (Waters electron triple-quadrupole system, described above) by MRM analysis as described in Table 2.

TABLE 2
Mass spectrometry parameters for CYP substrates.

| Enzyme | Probe Substrate Metabolite | MRM Transition | Cone voltage | Collision voltage | Mode |
|---|---|---|---|---|---|
| CYP1A2 | Acetaminophen | 152 > 110 | 30 | 20 | positive |
| CYP2A6 | Hydroxycoumarin | 161 > 133 | 30 | 15 | positive |
| CYP2B6 | Hydroxybupropion | 256 > 139 | 25 | 30 | positive |
| CYP2C8 | Desethylaminodiaquine | 328 > 283 | 30 | 35 | positive |
| CYP2C9 | Hydroxydiclofenac | 312 > 266 | 30 | 20 | positive |
| CYP2C19 | Hydroxyomeprazole | 362 > 214 | 30 | 20 | positive |
| CYP2D6 | Dextrophan | 258 > 199 | 30 | 20 | positive |
| CYP2E1 | HydroxychIorzoxazone | 184 > 120 | 30 | 20 | negative |
| CYP3A4 | Hydroxymidazolam | 342 > 324 | 30 | 20 | positive |

The measurement of CYPs 2B6, 2C8 and 2E1 activity was determined individually in human liver microsomes. The NA, positive control inhibitor, or vehicle control were pre-incubated in a reaction mixture as described above for other CYP enzymes bupropion (100 μM), amodiaquine (2 μM) and chlorzoxazone (100 μM) for CYPs 2B6, 2C8 and 2E1, respectively.

UGT Inhibition Assays.

Initial screening assays were performed as described above for the CYP assays using 1 μM or 10 μM of each of the NAs. The inhibitory effects of each agent against UGT2B10 was performed using the known UGT2B10-specific probe substrate, nicotine, in pooled human liver microsomes. Nicotine and alamethicin (Sigma-Aldrich, USA) were prepared as 20 mM stock solutions in and stored in aliquots at −80° C. Microsomes (0.25 mg/mL) were initially incubated with alamethicin (50 μg/mL protein) for 15 minutes in an ice bath. Pre-incubations containing 500 μM nicotine and 1 or 10 μM NA were subsequently performed at 37° C. in 50 mM Tris-HCl buffer (pH 7.5, in a final volume of 50 μL) and 10 mM MgCl₂ for 5 minutes. The reaction was initiated by the addition of 5 mM UDP-glucuronic acid (Sigma-Aldrich, USA), which proceeded for 60 minutes at 37° C. Reactions were terminated by the addition of 50 μL of stop solution (acetonitrile/methanol; 1:1, v/v). Samples were mixed on a vortex mixer and centrifuged at 13,500×g for 15 minutes at 4° C. The supernatant (˜75 μL) was then transferred to a clean tube, and the metabolite (nicotine-glucuronide) was detected using an ultra-pressure liquid chromatograph coupled to an electron triple-quadrupole mass spectrometer (Waters, described above) by MRM analysis, as described in Table 3.

TABLE 3
Mass spectrometry parameters for UGT substrates.

| Enzyme | Probe Substrate Metabolite | MRM Transition | Cone voltage | Collision voltage | Mode |
|---|---|---|---|---|---|
| UGT1A1 | β-Estradiol-3-glucuronide | 447 > 113 | 30 | 20 | positive |
| UGT1A4 | Trifluoperazine-N-glucuronide | 584 > 408 | 30 | 25 | positive |
| UGT1A6 | Serotonin-O-glucuronide | 353 > 177 | 30 | 20 | positive |
| UGT1A9 | Propofol-O-glucuronide | 353 > 177 | 40 | 25 | positive |
| UGT2B4 | Codeine-6-glucuronide | 476 > 300 | 40 | 35 | positive |
| UGT2B7 | Azidothymine-5-glucuronide | 442 > 125 | 40 | 30 | positive |
| UGT2B10 | Nicotine-glucuronide | 339 > 163 | 30 | 20 | positive |
| UGT2B15 | Lorazepam-glucuronide | 497 > 320 | 20 | 20 | positive |
| UGT2B17 | Testosterone-glucuronide | 465 > 289 | 30 | 25 | positive |

Inhibition assays were performed for UGT1A1, UGT1A4, UGT1A6, UGT1A9, UGT2B4, UGT2B7, UGT2B15 and UGT2B17 using estradiol, trifluoperazine, serotonin, propofol, codeine, lorazepam, and testosterone (all purchased from Sigma-Aldrich; St. Louis, Mo.), respectively, as the corresponding probe substrates; the concentrations used for individual substrates are indicated in Table 4.

TABLE 4
Probe substrates, concentrations and reference
inhibitors used in UGT assays.

| | | Substrate | |
|---|---|---|---|
| | Probe | Concentration | |
| Enzyme | Substrate | (μM) | Metabolite |
| UGT1A1 | Estradiol | 100 | β-Estradiol-3-glucuronide |
| UGT1A4 | Trifluoperazine | 25 | Trifluoperazine-N-glucuro- |
| | | | nide |
| UGT1A6 | Serotonin | 1000 | Serotonin-O-glucuronide |
| UGT1A9 | Propofol | 100 | Propofol-O-glucuronide |
| UGT2B4 | Codeine | 1000 | Codeine-6-glucuronide |
| UGT2B7 | Azidothymine | 600 | Azidothymine-5-glucuro- |
| | | | nide |
| UGT2B10 | Nicotine | 500 | Nicotine-glucuronide |
| UGT2B15 | Lorazepam | 200 | Lorazepam-glucuronide |
| UGT2B17 | Testosterone | 100 | Testosterone-glucuronide |

Determination of IC₅₀.

CYPs: For those agents that exhibited >50% inhibition of activity for any given CYP at <10 μM, IC₅₀ determinations were performed using multiple concentrations (0.005, 0.01, 0.1, 0.5, 1, 5, 10, 25 and 100 μM) of NA in 15 minutes/37° C. incubations. The final concentration of DMSO in the reaction mixture was always <0.1%. Peak areas corresponding to the probe metabolite were determined and the percentage of control activity was calculated by comparing the peak area in incubations containing the agents to the vehicle control sample in the absence of the NADPH regenerating system. IC₅₀ values were calculated using Graph Pad Prism software version 6.

UGTs: For those agents that exhibited >50% inhibition of activity for any given UGT enzyme at <1 mM concentration, IC₅₀ determinations were performed using multiple concentrations (25, 50, 100, 500, 1000, 2000 and 5000 μM) of NA in 60 minutes/37° C. incubations. The final concentration of DMSO in the reaction mixture was always <0.1%. Peak areas corresponding to the probe metabolite were determined and the percentage of control activity was calculated by comparing the peak area in incubations containing the agents to the vehicle control sample in the absence of UDP-glucuronic acid. IC₅₀ values were calculated using Graph Pad Prism software version 6.

Analytical Conditions.

As described above, probe substrate metabolites for all enzymatic reactions (CYPs and UGTs) were detected using an UPLC-MS/MS system (Waters Xevo TDQ Acquity UPLC-MS/MS system) by MRM analysis. The mobile phase consisted of solvent A (0.1% formic acid in water) and solvent B (100% methanol). Samples (2-5 μL) were injected onto an Acquity UPLC column (C18, 1.7 μM, 2.1×100 mm) from Waters Corp. The 6 minutes program was as follows: 95% A: 5% B (isocratic, 0-2 minutes), 5% A: 95% B (linear increase, 2-4 minutes), 5% A: 95% B (isocratic hold, 4-5 minutes) and 95% A: 5% B (re-equilibration, 5-6 minutes). The flow rate was 0.4 mL/minute and the column temperature was 40° C.

Alkyne Linker Agents.

A library of compounds were generated in an effort to delineate the initial pharmacophore of CYP2A6 using 5-substituted, 6-substituted and unsubstituted 3-heteroaromatic pyridine analogues of nicotine. In one embodiment the disclosed invention revealed the repetitive pharmacophore consisting of a 3-substituted pyridine ring connected to a primary methanamine via a linker consisting of an alkyne, a 1,5-substituted furan or a 1,5-substituted thiophene as the most active and highly selective inhibitors. The compound specifically consisting of the 3-pyridyl-alkyne-mathanamine was chosen as the reference agent. As described above, NAs were developed initially by focusing on altering the pyridine ring. All of the agents described previously were initially screened for anti-CYP2A6 activity at 1 μM and 10 μM. Given that the industry standard states any agent that exhibits an IC₅₀ of <10 μM may potentially be effective as a drug, agents that exhibited >40% inhibition at 10 μM in the initial screening were further assessed for their IC₅₀ against CYP2A6.

As shown in Table 5, agents C, E, H, I, J, K, L, M, N, O, P, and Q all exhibited IC₅₀ values of <10 μM, with agents C, H, I, J, L, M, N, and O exhibiting very effective IC₅₀ values of <1 μM, which is 11-fold less than the reported K_{M} (11 μM) for recombinant CYP2A6 against nicotine. The IC₅₀ of agents C, J, and L were 2.9-, 1.2-, and 1.2-fold lower, respectively, than that observed for the reference agent. Most interesting was the fact that, with the possible exception of agent E (with a moderate IC₅₀ value of 5.5), all of the agents with a moiety on the 4-position of the pyridine ring exhibited high inhibitory activity against CYP2A6. The 4-position was thus targeted for future studies and NA development.

TABLE 5
Inhibitory activity of nicotine analogues against

| Nicotine analogue | IC₅₀ against CYP2A6 (μM) |
|---|---|
| A | >10 |
| B | >10 |
| C | 0.055 |
| D | 10 |
| E | 5.5 |
| F | >10 |
| G | >10 |
| H | 0.83 |
| I | 0.44 |
| J | 0.13 |
| K | 3.4 |
| L | 0.13 |
| M | 0.56 |
| N | 0.67 |
| O | 0.59 |
| P | 3.6 |
| Q | 2.7 |
| ref agent | 0.16 |

The specificity of agents C, E, H, I, J, K, L, M, N, O, P, and Q for CYP2A6 was examined by assessing their inhibition of the eight major hepatic CYP enzymes known to metabolize the vast majority of drugs in humans (CYPs 1A2, 2B6, 2C8, 2C9, 2C19, 2D6, 2E1 and 3A4). For these studies, the IC₅₀ values of each agent were examined for each of the eight enzymes and compared to the IC₅₀ exhibited for that agent against CYP2A6. As shown in Table 6, the IC₅₀ ratio for any given CYP_{hepatilc}/CYP2A6 was >5.0 for all agents against all enzymes tested except for E, K, M, and N, which demonstrated selectivity ratios of <5.0 for at least two of the eight hepatic CYPs tested. Agent P demonstrated selectivity ratios of >5.0 for all tested CYPs except for CYP2D6. Agents C, J, L, O, and Q exhibited very high selectivity for CYP2A6, with IC₅₀ ratios of >40, >20, >25, >20, and >9, respectively, against all hepatic CYP enzymes. These data suggest that that several of these agents are highly inhibitory and selective for CYP2A6.

TABLE 6
IC₅₀ ratios for major hepatic CYP enzymes versus CYP2A6 for new agents.

| Nicotine | CYP3A4/ | CYP2D6/ | CYP2C9/ | CYP2C19/ | CYP1A2/ | CYP2B6/ | CYP2C8/ | CYP2E1/ |
|---|---|---|---|---|---|---|---|---|
| Analogue | CYP2A6 | CYP2A6 | CYP2A6 | CYP2A6 | CYP2A6 | CYP2A6 | CYP2A6 | CYP2A6 |
| C | 102 | 44 | 655 | 145 | 200 | 364 | 156 | 345 |
| E | >9.1 | >9.1 | >9.1 | 2.4 | 1.1 | 2.4 | >9.1 | 5.3 |
| H | >60 | 8.0 | >60 | 27 | >60 | 23 | 30 | 18 |
| 1 | >114 | 6.4 | 73 | 25 | >114 | 9.3 | 45 | 73 |
| J | 300 | 39 | 23 | 92 | 62 | 23 | 108 | 115 |
| K | >15 | >15 | 8.2 | 2.7 | 7.4 | 3.2 | >15 | 5.3 |
| L | >385 | 29 | >385 | 61 | 33 | 24 | >192 | 10 |
| M | 20 | 7.0 | 5.0 | 6.6 | 2.5 | 8.9 | 13 | 3.4 |
| N | 9.6 | 6.9 | 9.4 | 4.6 | 3.0 | 5.2 | 31 | 6.3 |
| O | 156 | 24 | 81 | 53 | 53 | 617 | >42 | 76 |
| P | >9.7 | 3.3 | >9.7 | >9.7 | 5.8 | >9.7 | >19 | >9.7 |
| Q | >9.3 | >9.3 | >9.3 | >9.3 | >9.3 | >9.3 | >19 | >9.3 |
| Ref Agent | 25 | 38 | 188 | 48 | 34 | 138 | 42 | 119 |

Thiophene and furan linker agents. We have also examined the IC₅₀ values of the thiophene- and furan-linker derivatives of agents C, H, I, J, K, M, N, O, P, and Q as well as the 4-chloro-thiophene derivative of agent L against CYP2A6. All of the thiophene and furan derivatives tested were effective inhibitors of CYP2A6, all exhibiting IC₅₀ values of ≤1.5 μM (Table 7). Agents T, U, V, W, AD, AE, and AF were very effective inhibitors of CYP2A6, exhibiting IC₅₀ values of ≤0.16 μM. In addition, agents R, S, T, U, AB, AE, AG, AI, AJ, AK, and AL were very selective for CYP2A6, exhibiting CYP_{hepatic}/CYP2A6 IC₅₀ ratios of <5.0 for no more than one tested hepatic CYP for any given agent (Table 8). Agents AG, AI, and AJ exhibited very high selectivity for CYP2A6, with IC₅₀ ratios of >10, >20, and >10, respectively, against all hepatic CYP enzymes tested.

TABLE 7
Inhibitory activity of thiophene and furan
nicotine analogues against CYP2A6.

| Nicotine analogue | IC₅₀ against CYP2A6 (μM) |
|---|---|
| R | 0.39 |
| S | 0.22 |
| T | 0.051 |
| U | 0.017 |
| V | 0.14 |
| W | 0.042 |
| X | 0.40 |
| Y | 0.41 |
| Z | 1.0 |
| AA | 0.55 |
| AB | 0.11 |
| AC | 0.29 |
| AD | 0.16 |
| AE | 0.11 |
| AF | 0.076 |
| AG | 0.38 |
| AH | 0.46 |
| AI | 0.30 |
| AJ | 0.33 |
| AK | 1.3 |
| AL | 1.2 |

TABLE 8
IC₅₀ ratios for major hepatic CYP enzymes versus CYP2A6
for thiophene and furan nicotine analogues.

| Nicotine | CYP3A4/ | CYP2D6/ | CYP2C9/ | CYP2C19/ | CYP1A2/ | CYP2B6/ | CYP2C8/ | CYP2E1/ |
|---|---|---|---|---|---|---|---|---|
| Analogue | CYP2A6 | CYP2A6 | CYP2A6 | CYP2A6 | CYP2A6 | CYP2A6 | CYP2A6 | CYP2A6 |
| R | 74 | 54 | 92 | 87 | 5.9 | 15 | 203 | 31 |
| S | 168 | 91 | 127 | 123 | 17 | 3.9 | 364 | 9.1 |
| T | 3549 | 59 | 1157 | 5.1 | 39 | 25 | 1431 | 18 |
| U | 765 | 265 | 941 | 49 | 235 | 5.5 | 88 | 14 |
| V | 35 | 12 | 136 | 14 | 2.6 | 1.9 | 30 | 14 |
| W | 102 | 64 | 202 | 26 | 4.5 | 2.1 | 52 | 36 |
| X | 13 | 2.1 | 6.5 | 11 | 0.73 | 0.45 | 13 | 18 |
| Y | 13 | 1.9 | 7.1 | 11 | 0.46 | 0.15 | 2.7 | 17 |
| Z | >25 | 12 | >50 | 4.1 | 1.3 | 3.9 | 16 | 5.9 |
| AA | >45 | 18 | >90 | 1.7 | 2.5 | 8.4 | 45 | 8.2 |
| AB | 136 | 71 | 182 | 6.8 | 17 | 7.4 | 227 | 47 |
| AC | 100 | 10 | 6.9 | 7.6 | 1.0 | 0.41 | 19 | 32 |
| AD | 44 | >312 | 28 | 8.8 | 1.3 | 0.46 | 24 | 19 |
| AE | 218 | 33 | 44 | 31 | 13 | 2.1 | >227 | 12 |
| AF | 68 | >658 | 80 | 11 | 3.6 | 0.82 | 63 | 72 |
| AG | 237 | 32 | 74 | 34 | 12 | 21 | >66 | 82 |
| AH | 91 | 35 | 50 | 20 | 3.5 | 2.6 | 6.5 | 80 |
| AI | 130 | 22 | >167 | 40 | 26 | 43 | >83 | >167 |
| AJ | 648 | 36 | 130 | 33 | 15 | 11 | >76 | 94 |
| AK | >19 | >19 | >19 | >19 | 5.8 | >19 | >38 | >38 |
| AL | 33 | 6.8 | 42 | 23 | 21 | 33 | >21 | >42 |

Inhibition of UGT2B10. A secondary goal was to examine whether any of the new agents could also selectively inhibit UGT2B10, the other major nicotine-metabolizing enzyme in humans. As shown in Table 9, several agents demonstrated inhibition of UGT2B10, with agents C and K exhibiting excellent inhibition with IC₅₀ values <60 μM, values that are 9-27-fold less than the K_{M} reported for recombinant UGT2B10 for nicotine itself [0.3-0.9 mM. Agents C and K exhibited IC₅₀ values that were less than the reference agent. Agents B, D, E, G, H and I exhibited little to no inhibition of UGT2B10, with IC₅₀ values >1 mM.

TABLE 9
Inhibitory activity of nicotine analogues against UGT2B10.

| Nicotine analogues | IC₅₀ against UGT2B10 (μM) |
|---|---|
| A | 770 |
| B | >1000 |
| C | 58 |
| D | >1000 |
| E | >1000 |
| F | 286 |
| G | >1000 |
| H | >1000 |
| I | >1000 |
| J | 175 |
| K | 33 |
| Ref Agent | 87 |

The IC₅₀ values observed for these agents against UGT2B10 are ˜10-1000-fold higher than those observed for CYP2A6. The UGTs are generally known as high-capacity, low-affinity enzymes; it is still not clear whether these differences in apparent IC₅₀ values could be due in part to the in vitro nature of the experimental system utilized. Previous studies (Guillemette C. Pharmacogen J. 2003; 3:136-158) suggest that the IC₅₀ values are a true reflection of in vivo enzyme kinetic differences between enzyme families.

Similar to that described above for CYPs, agents that exhibited IC₅₀ values <200 μM against UGT2B10 (agents C, J and K) were examined for their specificity against UGT2B10 by examining their inhibition of other major hepatic UGT enzymes (UGTs 1A1, 1A4, 1A6, 1A9, 2B4, 2B7, 2B10, 2B15 and 2B17). Similar to that observed for the reference agent (Table 10), none of the nicotine analogues except for agent K were highly selective for UGT2B10, with agent K exhibiting a UGThepatic/UGT2B10 IC50 ratio of >3.5 against all UGT enzymes tested.

TABLE 10
IC₅₀ ratios for major hepatic UGT enzymes versus UGT2B10 for new nicotine analogues.

| Nicotine | UGT1A1/ | UGT1A4/ | UGT1A6/ | UGT1A9/ | UGT2B4/ | UGT2B7/ | UGT2B15/ | UGT2B17/ |
|---|---|---|---|---|---|---|---|---|
| analogue | UGT2B10 | UGT2B10 | UGT2B10 | UGT2B10 | UGT2B10 | UGT2B10 | UGT2B10 | UGT2B10 |
| C | 0.7 | 1.6 | 6 | 10 | 13 | 17 | 17 | 2 |
| I | 0.5 | 0.9 | 2 | >1 | 0.7 | 1 | 1 | 1.2 |
| J | 1.6 | 2 | 5 | 3 | 1 | 6 | 6 | 18 |
| K | 4 | 7 | 29 | 5 | 3.5 | 13 | 30 | 26 |

In vitro stability studies. To assess the relative stability of each of the most active and selective NAs (IC₅₀<10 uM against CYP2A6, and exhibited CYP_{hepatic}/CYP2A6 IC₅₀ ratios of <5.0 for no more than one tested hepatic CYP), we incubated each agent at 37° C. with 12.5 μg of human liver microsomal protein containing 7 μM MgCl₂, 3 μL NADPH regenerating system (Corning), 10 μM nicotine analogue, and 50 mM KPO₄ buffer (pH 7.4) in a total volume of 25 μL. Aliquots of each reaction were taken at 0, 15, 30, 60 and 120 minutes, and agent amounts over time were calculated using UPLC-MS as described above by monitoring the exact mass of each agent. Half-lives were calculated using the one-phase exponential decay equation (GraphPad). Of the 19 top candidate agents tested, only agents C, H, R, S, and AB exhibited half-lives (T_{1/2}) of less than 25 minutes. Agents Q, T, AG, AI, AK, and AL were the most stable agents in incubations with human liver microsomes, all exhibiting a T_{1/2} of >90 minutes.

TABLE 11
T_{1/2} of 21 candidate nicotine analogues in human liver microsomes.

| Agent | T_{1/2} (min) |
|---|---|
| C | 8.4 |
| H | 9.8 |
| I | 39 |
| J | 48 |
| L | 28 |
| O | 46 |
| P | 36 |
| Q | >120 |
| R | 21 |
| S | 11 |
| T | >120 |
| U | 40 |
| AB | 11 |
| AE | 39 |
| AG | >120 |
| AI | >120 |
| AJ | 32 |
| AK | >120 |
| AL | >120 |
| ref agent 1 | >120 |

The data shows that several of the nicotine analogues including those that contain an alkene linker (agents C, H, I, J, L, O, P, and Q), a furan linker (agents R, T, AE, AG, AI, AK) or a thiophene linker (agents S, U, AB, AJ, and AL) exhibit high inhibitory activity against CYP2A6 without significant inhibitory activity against other major hepatic CYP enzymes. Of these 19 active and selective agents, 14 were stable, with 5 agents (Q, T, AG, AI, AK and AL) exhibiting half-lives in human liver microsomes of >120 minutes.

The inhibitory activity against UGT2B10. Agents C, J and K were inhibitory, but only agent K exhibited some specificity for this enzyme (not inhibiting other major hepatic UGTs). Studies are currently on-going examining the CYP2A6 furan- and thiophene-derivative agents described above for their activity against UGT2B10.

### Example 3: In Vivo Studies Examining the Toxicity and Efficacy of Novel Nicotine Analogues

Introduction: CYP2A6 is the major enzyme involved in the metabolism of nicotine, with it being the principal enzyme involved in the formation of cotinine from nicotine and 3-hydroxy (OH)-cotinine from cotinine (see schematic). Cotinine and its metabolites account for >70% of all urinary nicotine metabolites (including nicotine itself) in smokers. It is known that there are functional alleles in the CYP2A6 gene within the population that have little or no effect on human health. Therefore, the objective is to develop agents that can selectively target and inhibit CYP2A6 without cross-reacting with other enzymes or pathways, increasing the half-life of nicotine in smokers and enabling them to decrease their smoking habit, and potentially cease smoking altogether.

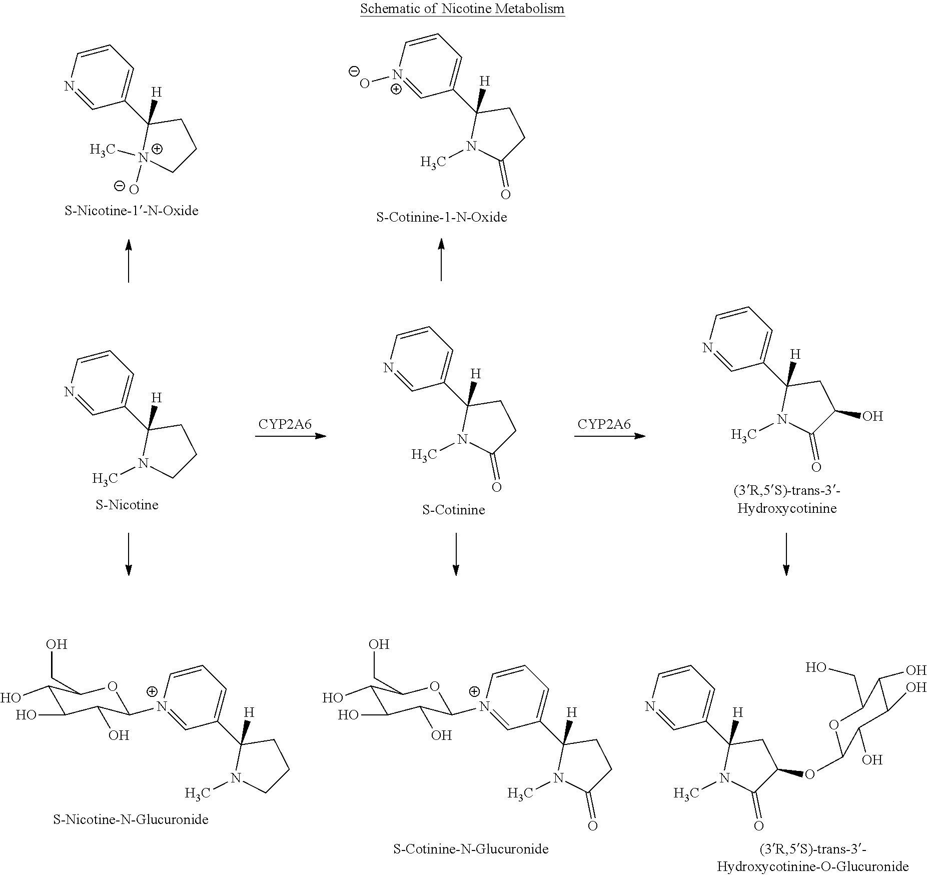

A. Dose Escalation Toxicity Study

Goal: Examine the toxicity of representative novel Compounds T and U in mice. Increasing doses of each compound were administered individually to mice, who were observed for acute toxic symptoms.

Experiment 1:

Methods

Mice: Fifteen C57BL/6J male mice (18-20 g, 4 weeks of age) were obtained from Jackson Laboratories. The animals were observed daily for one week prior to the start of the experiment, following the guidelines of the WSU-Spokane Vivarium standard operating procedures.

Compound formulation and preparation for dosing: Compounds T and U were formulated in USP grade PBS for i.p. injection. The stock solutions were:
T: 1.4 mg/mL (5.1 mM)
U: 0.5 mg/mL (1.7 mM)
   Dilutions of each stock solution were prepared one day before the start of the experiment, sterilized by filtration through a 0.2μ syringe filter, and stored at 4° C. Their pH was 7.0, and their osmolality was calculated prior to administration, and was within the expected isotonic value of 0.9%. Administered drug was diluted from the stock solution in PBS.

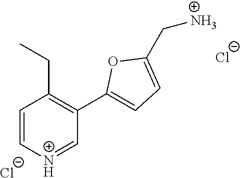

Chemical Formula: C₁₂H₁₆Cl₂N₂O
Molecular Weight: 275.17
   CYP2A6 IC₅₀: 0.051 μM; T_{1/2}=240 min
Compound T

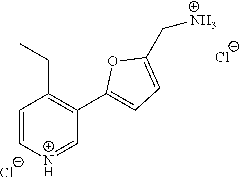

Chemical Formula: C₁₂H₁₆Cl₂N₂O
Molecular Weight: 275.17
   CYP2A6 IC₅₀: 0.017 μM; T_{1/2}=40 min
Compound U

Administration: Each mouse was administered i.p. with compound T, compound U or PBS (control) at a volume of 5 mL/kg mouse body weight (i.e., 0.1 mL/20 kg mouse), 5 mice/group. The dose concentrations were from 0.04× the IC₅₀ against CYP2A6 to 50× the CYP2A6 IC₅₀ (Table 12).

TABLE 12
Dose Escalation and Repeated Dose, Experiment 1.

| | | | | | | Relative to |
|---|---|---|---|---|---|---|
| | Group 1 | | Group 2 | | Group 3 | CYP2A6 |
| | (compound 5i) | | (Compound 6i) | | (Control) | IC₅₀ |
| Day 1 | 0.0015 | mg/kg | 0.0005 | mg/kg | 0.1 mL PBS | 0.04x |
| Day 2 | 0.003 | mg/kg | 0.001 | mg/kg | 0.1 mL PBS | 0.08x |
| Day 3 | 0.0055 | mg/kg | 0.002 | mg/kg | 0.1 mL PBS | 0.16x |
| Day 4 | 0.012 | mg/kg | 0.004 | mg/kg | 0.1 mL PBS | 0.31x |
| Day 5 | 0.023 | mg/kg | 0.0075 | mg/kg | 0.1 mL PBS | 0.63x |
| Day 6 | 0.045 | mg/kg | 0.015 | mg/kg | 0.1 mL PBS | 3.13x |
| Day 7 | 0.090 | mg/kg | 0.030 | mg/kg | 0.1 mL PBS | 6.25x |
| Day 8 | 0.18 | mg/kg | 0.065 | mg/kg | 0.1 mL PBS | 12.5x |
| Day 9 | 0.35 | mg/kg | 0.13 | mg/kg | 0.1 mL PBS | 25x |
| Day 10 | 0.70 | mg/kg | 0.25 | mg/kg | 0.1 mL PBS | 50x |
| Day 11 | 0.70 | mg/kg | 0.25 | mg/kg | 0.1 mL PBS | 50x |
| Day 12 | 0.70 | mg/kg | 0.25 | mg/kg | 0.1 mL PBS | 50x |
| Day 13 | 0.70 | mg/kg | 0.25 | mg/kg | 0.1 mL PBS | 50x |
| Day 14 | 0.70 | mg/kg | 0.25 | mg/kg | 0.1 mL PBS | 50x |

Animals were observed for acute toxic symptoms (including ataxia, tremors, hypoactivity, hunched body, abnormal breathing or mortality) post-injection for 15 min continually, followed by once per hour over a three-hour period. The animals had a 24 hour recovery time, followed by weighing, prior to the next i.p. injection. After the completion of 14 days of escalating dose injections, the mice were observed and their symptoms and weights were recorded for one additional week.

Results: No mice from any treatment group displayed any toxic symptoms.

Experiment 2. Data collected in feeding studies suggest that these compounds are tolerated at much higher doses when administered by oral gavage. Hence, a second dose escalation study was performed with increased doses, starting at 500× the IC₅₀ against CYP2A6 to 50,000× the CYP2A6 IC₅₀ (Table 2).

Methods: Mice were obtained and housed and the compounds were formulated and prepared for injection as described in Experiment 1. The injection volume remained the same at 100 μL for a 20 g mouse (5 mL/kg). However, the starting doses for the compounds in Experiment 2 were much higher, starting at 7 mg/kg for Compound T and 2.5 mg/kg for Compound U, increasing to 1400 mg/kg for Compound T and 500 mg/kg for Compound U. Each mouse was scheduled to receive a single injection, every day, for 6 days, as shown in Table 13.

TABLE 13
Dose Escalation and Toxicity, Experiment 2.

| | | | | | | Dose relative |
|---|---|---|---|---|---|---|
| | Group 1 | | Group 2 | | Group 3 | to CYP2A6 |
| | (Compound T) | | (Compound U) | | (Control) | IC₅₀ |
| Day 1 | 7 | mg/kg | 2.5 | mg/kg | PBS 0.1 mL | 500x |
| Day 2 | 35 | mg/kg | 12.5 | mg/kg | PBS 0.1 mL | 2500x |
| Day 3 | 70 | mg/kg | 25 | mg/kg | PBS 0.1 mL | 5000x |
| Day 4 | 350 | mg/kg | 125 | mg/kg | PBS 0.1 mL | 12,500x |
| Day 5 | 700 | mg/kg | 250 | mg/kg | PBS 0.1 mL | 25,000x |
| Day 6 | 1400 | mg/kg | 500 | mg/kg | PBS 0.1 mL | 50,000x |

The animals were observed for acute toxic symptoms as in Experiment 1.

Results:
1. For Compound T, mice began to show signs of physical toxicity, including tremors, at a dose of 35 mg/kg. However, the symptoms abated within 1 hour and the mice returned to normal behavior. The following day, the animals immediately died (within a few minutes) after administration of 70 mg/kg. Since a lethal dose was observed at 70 mg/kg on Day 4 of the experiment, the Day 5 and 6 doses were not performed for compound T.
2. Compound U was tolerated up to a dose of 125 mg/kg and found to be lethal at a dose of 250 mg/kg, showing similar symptoms as that described for the U group. Since a lethal dose was observed at 250 mg/kg on Day 5 of the experiment, the Day 6 dose was not performed for Compound U.

Conclusions: Both Compounds T and U were extremely well-tolerated at very high doses in mice.

B. Levels of Plasma Nicotine and its Metabolites In Vivo

Goal. In mice, nicotine is metabolized to cotinine (and cotinine to 3-OH-cotinine) by the mouse homologue of CYP2A6, an enzyme known as CYP2A5. The goal of the present experiment was to examine the effects of Compounds T and U on the levels of nicotine and its major metabolites, cotinine and 3-OH-cotinine, in the blood of mice injected with nicotine and compound. Altered levels of plasma nicotine (or cotinine and/or 3-OH-cotinne) would suggest an effect by these agents on CYP2A5 activity, an outcome that would be consistent with these agents also being inhibitory against CYP2A6 in humans.

Methods. Agents were prepared as described above for the dose escalation studies. Nicotine-tartrate was purchased from Sigma and prepared in USP-grade sterile PBS. Groups of 3-5 mice were injected with 1 mg/kg nicotine (100 uL) s.c. After 10 min, PBS (control group), compound T (7 mg/kg mouse body weight) or compound U (125 mg/kg mouse body weight) were injected by i.p. (intraperitoneal) or administered p.o. (oral) Blood (10 uL) was removed from the tail vein at various times post i.p. injection.

Blood was immediately processed, and plasma was isolated (2500×g for 5 min centrifugation at RT, saving top layer), and samples were frozen until analysis. For plasma metabolite analysis, samples (5 μL) were thawed on ice and 5 μL of internal standard (nicotine-d₄+cotinine-d₃; Toronto Res Chem) was added. Proteins were precipitated by the addition of 50 μL of LC-MS-grade methanol, samples were spun at 16,000×g for 15 min at 4° C., and supernatants were collected. Samples (5 μL) were injected onto a LC-MS (TQD, Sciex 6500) and metabolites were analyzed using a multiple reaction monitoring (MRM) method essentially as described previously.

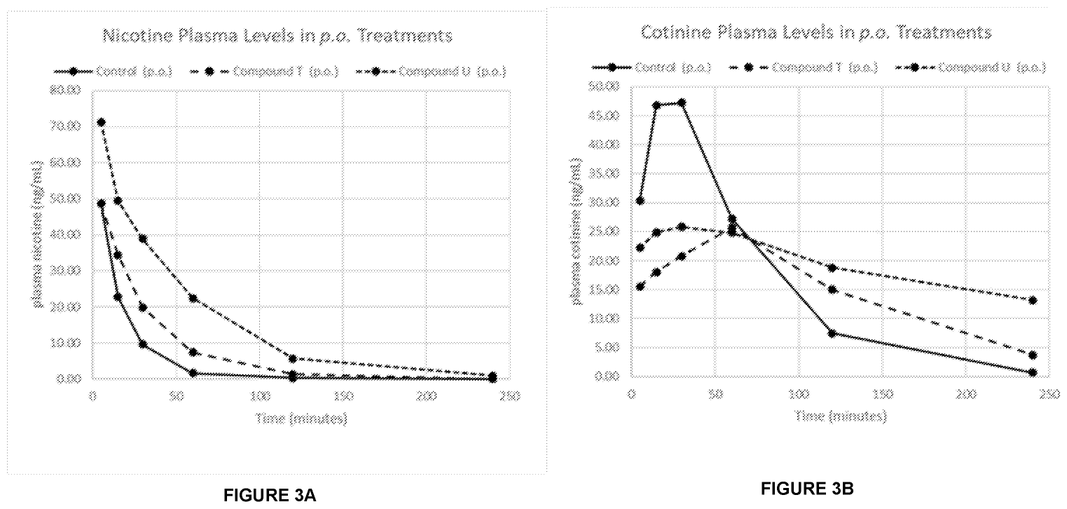

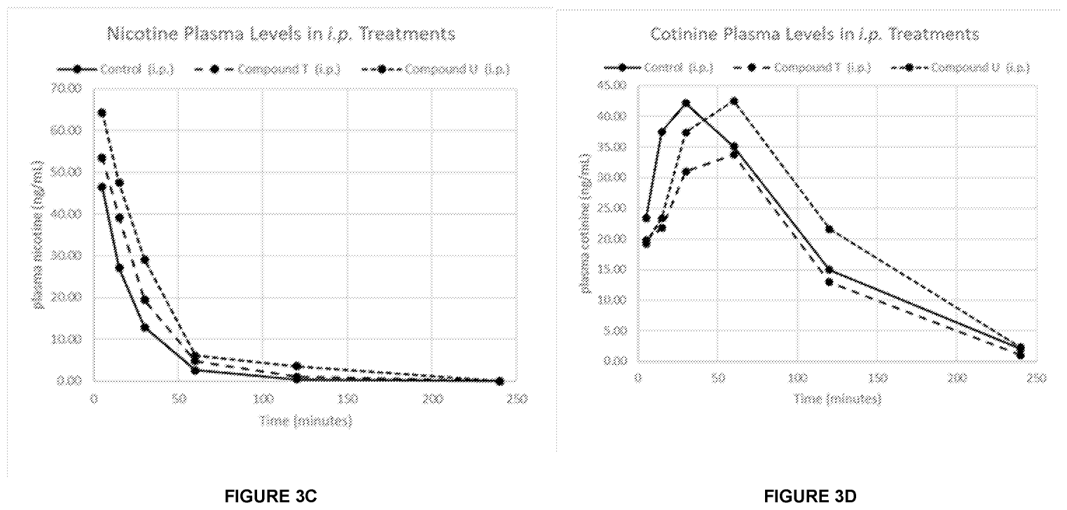

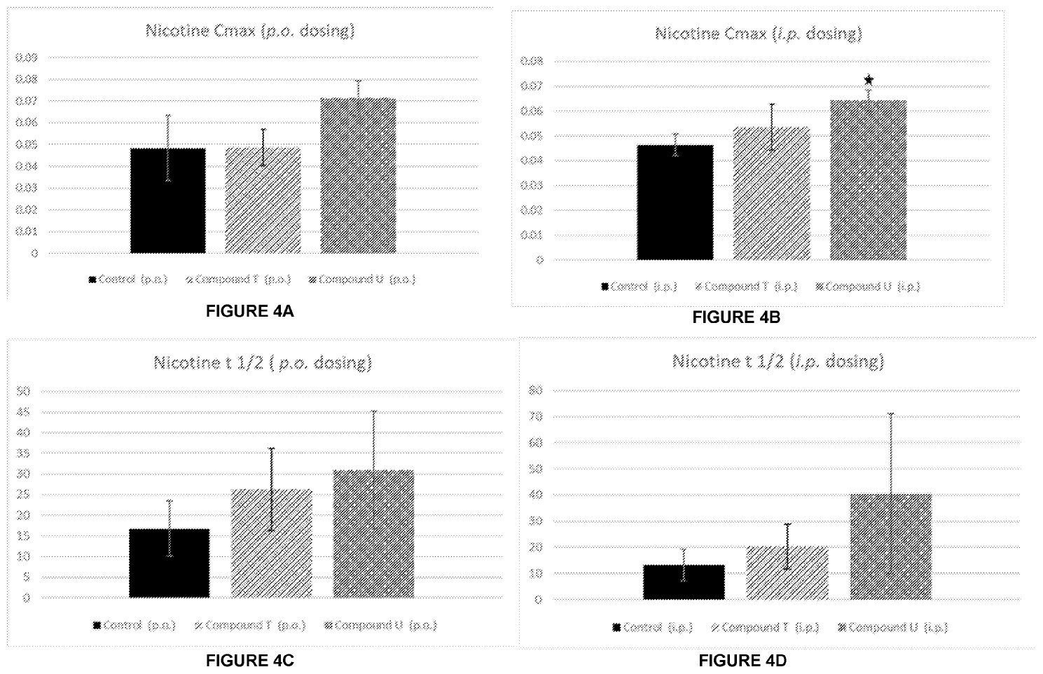

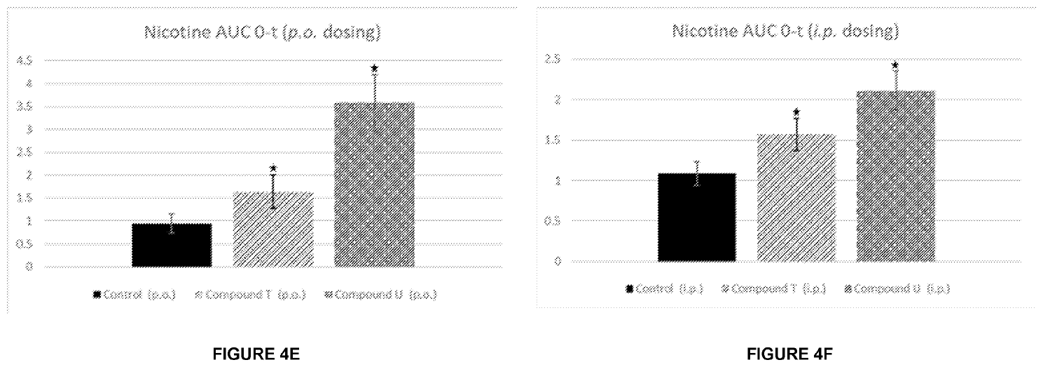

Results.
1. Levels of nicotine after p.o. administration. As shown in FIGS. 3A and 3B, the levels of nicotine are higher while the levels of cotinine are lower for up to 60 min post-administration in the mice treated with Compounds T or U as compared to control mice. Compound U appears to have a larger effect than Compound T.
2. Levels of cotinine after i.p. injection. As shown in FIGS. 3C and 3D, the same trends are observed after i.p. injection of compound as compared to that observed after p.o. administration.
3. Pharmacokinetic data. Table 14 (FIGS. 4A and 4B) shows that the Cₘₐₓ for nicotine was higher for Compound U as compared to controls after either p.o. administration or after i.p. injection. The effect of Compound T on the nicotine Cₘₐₓ was less clear. The t_{1/2} for nicotine (see Table 14 and FIGS. 4C and 4D) appeared to be higher for both Compounds T and U as compared to controls after either p.o. administration or after i.p. injection, but there was some variability between mice. The AUC 0-t for nicotine was higher for Compound U as compared to controls after either p.o. administration or after i.p. injection (see FIGS. 4E and 4F). A similar but lesser effect was also observed for Compound U. A virtually identical pattern was observed for AUC-0-inf (see Table 14).

TABLE 14
Pharmacokinetic data of plasma nicotine and cotinine levels
after administration of Compound T or U v. control mice.

| | | COTININE | | | | NICOTINE | | | |
|---|---|---|---|---|---|---|---|---|---|
| Sample | | | AUC | t ½ | AUC | | AUC | t ½ | AUC |
| Name | | Cmax | 0-t* | (min) | 0-inf | Cmax | 0-t* | (min) | 0-inf |
| p.o control | MEAN | 0.05 | 3.81 | 34.05 | 3.85 | 0.05 | 0.95 | 16.77 | 0.96 |
| | SD | 0.01 | 0.64 | 1.49 | 0.65 | 0.02 | 0.22 | 6.68 | 0.22 |
| Cpd T (p.o) | MEAN | 0.03 | 3.55 | 63.75 | 3.92 | 0.05 | 1.64 | 26.23 | 1.68 |
| | SD | 0.00 | 0.11 | 15.55 | 0.30 | 0.01 | 0.36 | 9.96 | 0.32 |
| Cpd U (p.o) | MEAN | 0.03 | 4.66 | 375.27 | 11.21 | 0.07 | 3.58 | 30.98 | 3.62 |
| | SD | 0.01 | 1.3 | 360.19 | 4.12 | 0.01 | 0.62 | 14.22 | 0.64 |
| i.p control | MEAN | 0.04 | 4.6 | 43.36 | 4.77 | 0.05 | 1.09 | 13.24 | 1.10 |
| | SD | 0.01 | 0.6 | 8.08 | 0.65 | 0.00 | 0.15 | 6.15 | 0.15 |
| Cpd T (i.p) | MEAN | 0.03 | 3.8 | 33.06 | 3.92 | 0.05 | 1.57 | 20.34 | 1.61 |
| | SD | 0.00 | 0.3 | 10.92 | 0.36 | 0.01 | 0.20 | 8.60 | 0.24 |
| Cpd U (i.p) | MEAN | 0.04 | 5.2 | 42.14 | 5.42 | 0.06 | 2.11 | 40.55 | 2.40 |
| | SD | 0.01 | 0.9 | 8.60 | 0.86 | 0.00 | 0.24 | 30.64 | 0.14 |

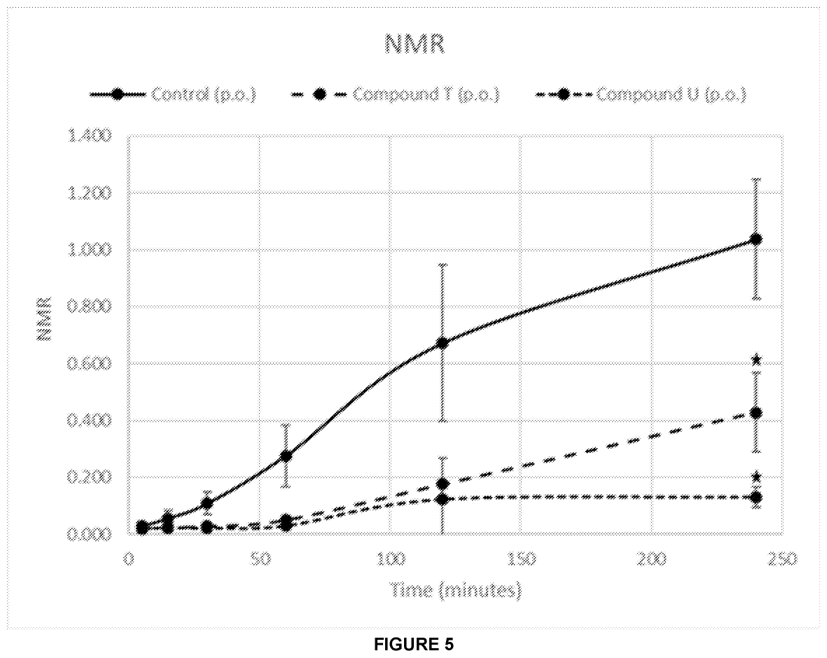

4. Nicotine Metabolite Ratio (NMR) for U-treated mice vs. control mice, administered p.o. While cotinine may be produced less in Compound U-treated mice due to inhibition of CYP2A5 activity, whatever cotinine is produced could potentially be inhibited from metabolism to 3-OH-cotinine, which is also catalyzed by CYP2A5. Cotinine and 3-OH-cotinine were assayed, and the ratio of 3-OH-cotinine/cotinine (NMR) was obtained. The higher the ratio, the more 3-OH-cotinine is being formed, hence the higher the CYP2A5 activity in mice. FIG. 5 shows that the NMR ratio, and therefore CYP2A5 activity, was higher in the control mice than in the Compound U-treated mice. This indicates that Compound U inhibited CYP2A5 activity in these mice. CYP2A5 is a stable marker for CYP2A6. Therefore, Compound U would have the same effect on CYP2A6 activity.

Conclusions. Both Compounds T and U appear to decrease the metabolism of both nicotine and cotinine in mice, indicating that the mouse homologue of CYP2A6, CYP2A5, is being inhibited by these agents.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The subject matter described above is provided by way of illustration only and should not be construed as limiting. Various modifications and changes may be made to the subject matter described herein without following the example embodiments and applications illustrated and described, and without departing from the true spirit and scope of the present disclosure, which is set forth in the following claims.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. A compound of Formula I, 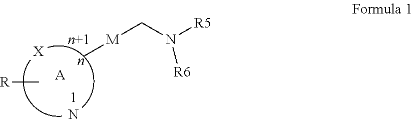
wherein,
A is a heterocycle comprising X, N, and one to 5 carbon atoms, and N is at position 1 on the heterocycle;
X is C, N, O, or S and X is at any position on the heterocycle that is not occupied by N or a carbon atom bonded to M;
M is a linker and M is bonded to the carbon atom on the heterocycle at position n, wherein n is 2, 3, 4, 5, or 6;
R is one or more substituents on A, and at least one R is at position n+1;
each R is the same or different and is independently alkyl, cycloalkyl, aminoalkyl, aralkyl, alkoxy, alkoxyalkyl, cycloalkenyl, alkynyl, cycloalkynyl, acyl, acylalkyl, acyloxy, acyloxyalkyl, heterocycle, aryl, heteroaryl, heteroaralkyl, heteroaralkyloxy, aroyl, aroylalkyl, aryloxy, aryloxyalkyl, halogen, haloalkyl, cyano, cyanoalkyl, nitro, nitroalkyl, carboxyl, carboxylalkyl, amino, aminoalkyl, aminocarbonyl, aminocarbonylalkyl, carbamoylalkyl, carbamoylalkoxy, iminoalkyl, imidoalkyl, alkoxycarbonyl, alkoxycarbonylalkyl, alkylamino, alkylaminoalkyl, dialkylamino, dialkylaminoalkyl, arylamino, arylaminoalkyl, hydroxy, hydroxyalkyl, isocyano, isocyanoalkyl, isothiocyano, isothiocyanoalkyl, oximinoalkoxy, morpholino, morpholinoalkyl, azido, azidoalkyl, formyl, formylalkyl, alkylthio, alkylthioalkyl, alkylsulfinyl, alkylsulfinylalkyl, alkylsulfonyl, alkylsulfonylalkyl, aminosulfonyl, arylsulfonyl, N-alkyl-N-arylaminosulfonyl, heteroatom, or heteroatom-containing group, and wherein each is optionally substituted by one or more substituents; and
R5 and R6 are the same or different and are independently alkyl, cycloalkyl, aminoalkyl, aralkyl, alkoxy, alkoxyalkyl, cycloalkenyl, alkynyl, cycloalkynyl, acyl, acylalkyl, acyloxy, acyloxyalkyl, heterocycle, aryl, heteroaryl, heteroaralkyl, heteroaralkyloxy, aroyl, aroylalkyl, aryloxy, aryloxyalkyl, hydrogen, halogen, haloalkyl, cyano, cyanoalkyl, nitro, nitroalkyl, carboxyl, carboxylalkyl, amino, aminoalkyl, aminocarbonyl, aminocarbonylalkyl, carbamoylalkyl, carbamoylalkoxy, iminoalkyl, imidoalkyl, alkoxycarbonyl, alkoxycarbonylalkyl, alkylamino, alkylaminoalkyl, dialkylamino, dialkylaminoalkyl, arylamino, arylaminoalkyl, hydroxy, hydroxyalkyl, isocyano, isocyanoalkyl, isothiocyano, isothiocyanoalkyl, oximinoalkoxy, morpholino, morpholinoalkyl, azido, azidoalkyl, formyl, formylalkyl, alkylthio, alkylthioalkyl, alkylsulfinyl, alkylsulfinylalkyl, alkylsulfonyl, alkylsulfonylalkyl, aminosulfonyl, arylsulfonyl, N-alkyl-N-arylaminosulfonyl, heteroatom, or heteroatom-containing group, wherein each is optionally substituted by one or more substituents.

2. The compound of claim 1, wherein M is,

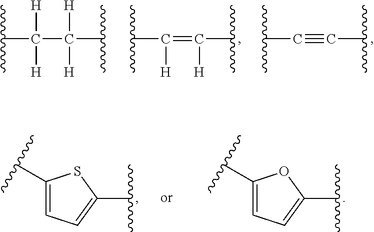

3. The compound of claim 1, wherein,
A is a 5- or 6-membered heterocycle; and
R is a lower alkyl, aryl, heteroaryl, cycloalkyl, cycloakenyl, cycloalkynyl, or spirocyclic.

4. The compound of claim 1, wherein A is pyridine, pyrazole, imidazole, isoxazole, oxazole, isothiazole, thiazole, pyrimidine, pyridazine, pyrazine, thiophene, or furan.

5. The compound of claim 1, wherein A is a pyridine and R is positioned para to N.

6. The compound of claim 1, wherein the compound has Formula II, 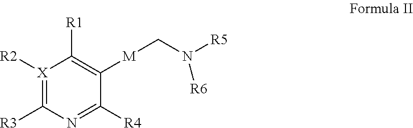
R1 is halogen, alkyl, aryl, aralkyl, heteroaryl, alkenyl, alkynyl, cycloalkyl, heterocycle, or heteroatom-containing group, wherein the alkyl, aryl, aralkyl, heteroaryl, alkenyl, alkynyl, cycloalkyl, heterocycle, or heteroatom-containing group is optionally substituted by one or more substituents;
R2, R3, and R4 are the same or different and are independently halogen, hydrogen, alkyl, aryl, aralkyl, heteroaryl, alkenyl, alkynyl, cycloalkyl, heterocycle, or heteroatom-containing group, wherein the alkyl, aryl, aralkyl, heteroaryl, alkenyl, alkynyl, cycloalkyl, heterocycle, or heteroatom-containing group is optionally substituted by one or more substituents;
X is N or C;
M is an independently 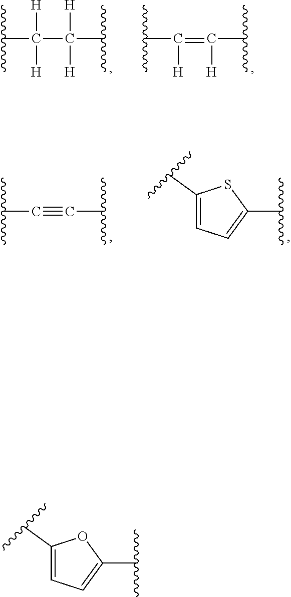
and
R5 and R6 are the same or different and are independently hydrogen, halogen, alkyl, aryl, aralkyl, heteroaryl, alkenyl, alkynyl, cycloalkyl, heterocycle, heteroatom-containing group, or a protecting group, wherein alkyl, aryl, aralkyl, heteroaryl, alkenyl, alkynyl, cycloalkyl, heterocycle, heteroatom-containing group, or protecting group is optionally substituted by one or more substituents.

7. The compound of claim 6, wherein,
R1 is a lower alkyl; and
each of R2, R3, and R4 is hydrogen.

8. A salt or solvate of the compound of claim 1, or a solvate of the salt of the compound of claim 1.

9. A composition, wherein the composition comprises the compound, or a salt or solvate of claim 1 and a carrier.

10. The composition of claim 9, wherein the composition is a pharmaceutical composition and the carrier is a pharmaceutically acceptable carrier.

11. A method of treating, preventing, or reducing the risk of developing a disease or disorder, wherein the method comprises administering a therapeutically effective amount the pharmaceutical composition of claim 10 to the subject.

12. The method of claim 11, wherein treating, preventing, or reducing the risk of developing a disease or disorder comprises blocking CYP2A6- and/or UGT2B10-mediated nicotine metabolism.

13. The method of claim 11, wherein the disease or disorder is nicotine addiction, cancer, alcoholism, neurodegenerative disease, psychiatric disorder, cardiovascular disease, blindness, cataracts, periodontitis, pneumonia, chronic obstructive pulmonary disease, asthma, diabetes, reduced fertility, ectopic pregnancy, erectile dysfunction, rheumatoid arthritis, or alcoholism.

14. The method of claim 13,
wherein the cancer is oropharyngeal cancer, laryngeal cancer, esophageal cancer, tracheal cancer, bronchial cancer, lung cancer, acute myeloid leukemia, stomach cancer, liver cancer, pancreatic cancer, kidney cancer, ureter cancer, cervical cancer, bladder cancer, or colorectal cancer;
wherein the psychiatric disorder is anxiety disorders such as post-traumatic stress disorder, bipolar disorder, generalized anxiety disorder, obsessive-compulsive disorder, panic disorder, separation anxiety, social anxiety disorder, or attention deficit disorder; or
wherein the cardiovascular disease is stroke, myocardial infarction, aortic aneurysm, or atherosclerosis.

15.-17. (canceled)

18. A method of treating a subject that would benefit from blocking CYP2A6- and/or UGT2B10-meditated nicotine metabolism, wherein the method comprises administering a therapeutically effective amount of the pharmaceutical composition of claim 10 to a subject in need thereof.

19. A method of blocking CYP2A6- and/or UGT2B10-mediated nicotine metabolism, wherein the method comprises administering the pharmaceutical composition of claim 10 to cells associated with cells overexpressing CYP2A6- and/or UGT2B10-mediated nicotine metabolism and assaying for nicotine metabolites.

20. The method of claim 19, wherein the cells are an in vitro sample of cells or an in vivo sample of cells.

21. The method of claim 19, wherein the cells are human liver microsomes.

22. A method of blocking CYP2A13 and/or CYP2A6 activation of pro-carcinogens, wherein the method comprises administering the pharmaceutical composition of claim 10 to a subject in need thereof.

23. The method of claim 22, wherein the subject is diagnosed with cancer or is at risk of developing cancer.

24. The method of claim 23, wherein the subject has lung cancer or has a risk of developing lung cancer.

25.-27. (canceled)
